(19) United States
(12) Patent Application Publication
(10) Pub. No.: US 2005/0142133 A1
(43) Pub. Date: Jun. 30, 2005

(54) Optimized proteins that target the epidermal growth factor receptor

(75) Inventors: Gregory Alan Lazar, West Covina, CA (US); Wei Dang, Pasadena, CA (US); John R. Desjarlais, Pasadena, CA (US); Philip W. Hammond, Sierra Madre, CA (US); Jost Vielmetter, Altadena, CA (US)

Correspondence Address: Robin M. Silva;Dorsey & Whitney LLP, Intellectual Property Department, Four Embarcadero Center, Suite 3400, San Francisco, CA, 94111-4187, US

(73) Assignee: Xencor, Inc.

(21) Appl. No.: 11/004,054

(22) Filed: Dec. 3, 2004

Related U.S. Application Data
(60) Provisional application No. 60/526,799, filed on Dec. 3, 2003.

(51) Int. Cl. A61K 39/395; C07K 16/28

(52) U.S. Cl. 424/143.1; 530/388.22

(57) ABSTRACT

The present invention relates to optimized proteins that target the Epidermal Growth Factor Receptor (EGFR), and their application, particularly for therapeutic purposes.

### CROSS REFERENCES TO RELATED APPLICATIONS

This application claims benefit under 35 U.S.C. §119(e) to U.S. Ser. No. 60/526,799, filed Dec. 3, 2003, incorporated herein by reference in its entirety.

### FIELD OF THE INVENTION

The present invention relates to optimized proteins that target the Epidermal Growth Factor Receptor (EGFR), and their application, particularly for therapeutic purposes.

### BACKGROUND OF THE INVENTION

Epidermal growth factor receptor (EGFR, also referred to as ErbB-1 or HER-1) is a 170 kDa transmembrane glycoprotein expressed primarily in cells of epithelial origin. EGFR is a member of the ErbB family of receptor tyrosine kinases (RTKs), which includes EGFR (also referred to as ErB-1 or HER1), ErbB-2 (HER2 or Neu), ErbB-3 (HER3), and ErbB-4 (HER4). The ErbB RTKs all share the same basic structure—an extracellular ligand binding domain, an intracytoplasmic protein tyrosine kinase with a regulatory carboxyl terminal segment, and a transmembrane domain. A number of ligands that bind EGFR have been characterized, including epidermal growth factor (EGF), transforming growth factor-α (TGFα), amphiregulin, heparin-binding EGF-like growth factor, betacellulin, epiregulin, CRIPTO (teratocarcinoma-derived growth factor), and vaccinnia virus growth factor (Salomon et al., 1995, Crit. Rev. Oncol. Hematol. 19:183-232). EGFR is overexpressed as compared to normal cells in a variety of human cancers, including head and neck, lung, breast, colon, and other solid tumors, and its overexpression is correlated with poor prognososis in cancer patients. Its role in cancer makes EGFR a target for anti-cancer therapy, and a number of small molecule drugs and protein therapeutics are approved and in trials for the treatment of cancers overexpressing EGFR (Khalil et al., 2003, Expert Rev. Anticancer Ther. 3(3):367-380; de Bono & Rawinski, 2002, Trends in Molecular Medicine 8(4):S19-S26; Alroy & Yarden, 1997, FEBS Letters 410:83-86).

A common class of therapeutic proteins are monoclonal antibodies. A number of favorable properties of antibodies, including but not limited to specificity for target, ability to mediate immune effector mechanisms, and long half-life in serum, make antibodies powerful therapeutics. A number of antibodies that target EGFR are approved or in clinical trials for the treatment of a variety of cancers, including but not limited to Cetuximab (Erbitux®, Imclone) (U.S. Pat. No. 4,943, 533; PCT WO 96/40210); ABX-EGF (Abgenix-Immunex-Amgen) (U.S. Pat. No. 6,235,883; Yang et al., 2001, Crit. Rev. Oncol. Hematol. 38:17-23); HuMax-EGFr (Genmab) (U.S. Ser. No. 10/172,317), 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer).

Antibodies are immunological proteins that bind a specific antigen. In most mammals, including humans and mice, antibodies are constructed from paired heavy and light polypeptide chains. Each chain is made up of individual immunoglobulin (Ig) domains, and thus the generic term immunoglobulin is used for such proteins. Each chain is made up of two distinct regions, referred to as the variable and constant regions. The light and heavy chain variable regions show significant sequence diversity between antibodies, and are responsible for binding the target antigen. The constant regions show less sequence diversity, and are responsible for binding a number of natural proteins to elicit important biochemical events. In humans there are five different classes of antibodies including IgA (which includes subclasses IgA1 and IgA2), IgD, IgE, IgG (which includes subclasses IgG1, IgG2, IgG3, and IgG4), and IgM. The distinguishing features between these antibody classes are their constant regions, although subtler differences may exist in the V region. IgG antibodies are tetrameric proteins composed of two heavy chains and two light chains. The IgG heavy chain is composed of four immunoglobulin domains linked from N- to C-terminus in the order V_{H}-CH1-CH2-CH3, referring to the heavy chain variable domain, heavy chain constant domain 1, heavy chain constant domain 2, and heavy chain constant domain 3 respectively (also referred to as V_{H}-Cγ1-Cγ2-Cγ3, referring to the heavy chain variable domain, constant gamma 1 domain, constant gamma 2 domain, and constant gamma 3 domain respectively). The IgG light chain is composed of two immunoglobulin domains linked from N- to C-terminus in the order V_{L}-C_{L}, referring to the light chain variable domain and the light chain constant domain respectively.

The variable region of an antibody contains the antigen binding determinants of the molecule, and thus determines the specificity of an antibody for its target antigen. The variable region is so named because it is the most distinct in sequence from other antibodies within the same class. The majority of sequence variability occurs in the complementarity determining regions (CDRs). There are 6 CDRs total, three each per heavy and light chain, designated V_{H} CDR1, V_{H} CDR2, V_{H} CDR3, V_{L} CDR1, V_{L} CDR2, and V_{L} CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens. A number of high-resolution structures are available for a variety of variable region fragments from different organisms, some unbound and some in complex with antigen. The sequence and structural features of antibody variable regions are well characterized (Morea et al., 1997, Biophys Chem 68:9-16; Morea et al., 2000, Methods 20:267-279), and the conserved features of antibodies have enabled the development of a wealth of antibody engineering techniques (Maynard et al., 2000, Annu Rev Biomed Eng 2:339-376). Fragments comprising the variable region can exist in the absence of other regions of the antibody, including for example the antigen binding fragment (Fab) comprising V_{H}-Cγ1 and VH₋C_{L}, the variable fragment (Fv) comprising V_{H} and V_{L}, the single chain variable fragment (scFv) comprising V_{H} and V_{L} linked together in the same chain, as well as a variety of other variable region fragments (Little et al., 2000, Immunol Today 21:364-370).

The Fc region of an antibody interacts with a number of Fc receptors and ligands, imparting an array of important functional capabilities referred to as effector functions. For IgG the Fc region comprises Ig domains Cγ2 and Cγ3 and the N-terminal hinge leading into Cγ2. An important family of Fc receptors for the IgG class are the Fc gamma receptors (FcγRs). These receptors mediate communication between antibodies and the cellular arm of the immune system (Raghavan et al., 1996, Annu Rev Cell Dev Biol 12:181-220; Ravetch et al., 2001, Annu Rev Immunol 19:275-290). In humans this protein family includes FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65). These receptors typically have an extracellular domain that mediates binding to Fc, a membrane spanning region, and an intracellular domain that may mediate some signaling event within the cell. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and γγ T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. The cell-mediated reaction wherein nonspecific cytotoxic cells that express FcγRs recognize bound antibody on a target cell and subsequently cause lysis of the target cell is referred to as antibody dependent cell-mediated cytotoxicity (ADCC) (Raghavan et al., 1996, Annu Rev Cell Dev Biol 12:181-220; Ghetie et al., 2000, Annu Rev Immunol 18:739-766; Ravetch et al., 2001, Annu Rev Immunol 19:275-290). The cell-mediated reaction wherein nonspecific cytotoxic cells that express FcγRs recognize bound antibody on a target cell and subsequently cause phagocytosis of the target cell is referred to as antibody dependent cell-mediated phagocytosis (ADCP).

The different IgG subclasses have different affinities for the FcγRs, with IgG1 and IgG3 typically binding substantially better to the receptors than IgG2 and IgG4 (Jefferis et al., 2002, Immunol Lett 82:57-65). All FcγRs bind the same region on IgG Fc, yet with different affinities: the high affinity binder FcγRI has a Kd for IgG1 of 10⁻⁸ M⁻¹, whereas the low affinity receptors FcγRII and FcγRIII generally bind at 10⁻⁶ and 10⁻⁵ respectively. The extracellular domains of FcγRIIIa and FcγRIIIb are 96% identical, however FcγRIIIb does not have a intracellular signaling domain. Furthermore, whereas FcγRI, FcγRIIa/c, and FcγRIIIa are positive regulators of immune complex-triggered activation, characterized by having an intracellular domain that has an immunoreceptor tyrosine-based activation motif (ITAM), FcγRIIb has an immunoreceptor tyrosine-based inhibition motif (ITIM) and is therefore inhibitory. Thus the former are referred to as activation receptors, and FcγRIIb is referred to as an inhibitory receptor. The receptors also differ in expression pattern and levels on different immune cells. Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. This difference in affinity, and presumably its effect on ADCC and/or ADCP, has been shown to be a significant determinant of the efficacy of the anti-CD20 antibody rituximab (Rituxan®, a registered trademark of IDEC Pharmaceuticals Corporation). Patients with the V158 allotype respond favorably to rituximab treatment; however, patients with the lower affinity F158 allotype respond poorly (Cartron et al., 2002, Blood 99:754-758). Approximately 10-20% of humans are V158/V158 homozygous, 45% are V158/F158 heterozygous, and 35-45% of humans are F158/F158 homozygous (Lehrnbecher et al., 1999, Blood 94:4220-4232; Cartron et al., 2002, Blood 99:754-758). Thus 80-90% of humans are poor responders, that is they have at least one allele of the F158 FcγRIIIa.

An overlapping but separate site on Fc, serves as the interface for the complement protein C1q. In the same way that Fc/FcγR binding mediates ADCC, Fc/C1q binding mediates complement dependent cytotoxicity (CDC). A site on Fc between the Cγ2 and Cγ3 domains, mediates interaction with the neonatal receptor FcRn, the binding of which recycles endocytosed antibody from the endosome back to the bloodstream (Raghavan et al, 1996, Annu Rev Cell Dev Biol 12:181-220; Ghetie et al., 2000, Annu Rev Immunol 18:739-766). This process, coupled with preclusion of kidney filtration due to the large size of the full length molecule, results in favorable antibody serum half-lives ranging from one to three weeks. Binding of Fc to FcRn also plays a key role in antibody transport. The binding site for FcRn on Fc is also the site at which the bacterial proteins A and G bind. The tight binding by these proteins is typically exploited as a means to purify antibodies by employing protein A or protein G affinity chromatography during protein purification. A key feature of the Fc region is the conserved N-linked glycosylation that occurs at N297. This carbohydrate, or oligosaccharide as it is sometimes referred, plays a critical structural and functional role for the antibody, and is one of the principle reasons that antibodies must be produced using mammalian expression systems.

In addition to antibodies, an antibody-like protein that is finding an expanding role in research and therapy is the Fc fusion (Chamow et al., 1996, Trends Biotechnol 14:52-60; Ashkenazi et al., 1997, Curr Opin Immunol 9:195-200). An Fc fusion is a protein wherein one or more polypeptides is operably linked to Fc. An Fc fusion combines the Fc region of an antibody, and thus its favorable effector functions and pharmacokinetics, with the target-binding region of a receptor, ligand, or some other protein or protein domain. The role of the latter is to mediate target recognition, and thus it is functionally analogous to the antibody variable region. Because of the structural and functional overlap of Fc fusions with antibodies, the discussion on antibodies in the present invention extends directly to Fc fusions.

There are a number of possible mechanisms by which antibodies destroy tumor cells, including anti-proliferation via blockage of needed growth pathways, intracellular signaling leading to apoptosis, enhanced down regulation and/or turnover of receptors, CDC, ADCC, ADCP, and promotion of an adaptive immune response (Cragg et al., 1999, Curr Opin Immunol 11:541-547; Glennie et al., 2000, Immunol Today 21:403-410). Anti-tumor efficacy may be due to a combination of these mechanisms, and their relative importance in clinical therapy appears to be cancer dependent. Despite this arsenal of anti-tumor weapons, the potency of antibodies as anti-cancer agents is unsatisfactory, particularly given their high cost. Patient tumor response data show that monoclonal antibodies provide only a small improvement in therapeutic success over normal single-agent cytotoxic chemotherapeutics. For example, just half of all relapsed low-grade non-Hodgkin's lymphoma patients respond to the anti-CD20 antibody rituximab (McLaughlin et al., 1998, J Clin Oncol 16:2825-2833). Of 166 clinical patients, 6% showed a complete response and 42% showed a partial response, with median response duration of approximately 12 months. Trastuzumab (Herceptin®, a registered trademark of Genentech), an anti-HER2/neu antibody for treatment of metastatic breast cancer, has less efficacy. The overall response rate using trastuzumab for the 222 patients tested was only 15%, with 8 complete and 26 partial responses and a median response duration and survival of 9 to 13 months (Cobleigh et al., 1999, J Clin Oncol 17:2639-2648). Despite the fact that EGFR is expressed on up to 77 percent of colorectal cancer tumors, combination therapy with cetuximab (Erbitux®, Imclone/BMS) had an objective response rate of 22.5% with a median duration of response of 84 days (Saltz et al., 2001, Proc.Am. Soc. Clin. Oncol. 20, 3a); results of the cetuximab single agent treatment group were even worse. Currently for anticancer therapy, any small improvement in mortality rate defines success. Thus there is a significant need to enhance the capacity of antibodies to destroy targeted cancer cells.

A promising means for enhancing the anti-tumor potency of antibodies is via enhancement of their ability to mediate cytotoxic effector functions such as ADCC, ADCP, and CDC. The importance of FcγR-mediated effector functions for the anti-cancer activity of antibodies has been demonstrated in mice (Clynes et al., 1998, Proc Natl Acad Sci USA 95:652-656; Clynes et al., 2000, Nat Med 6:443-446), and the affinity of interaction between Fc and certain FcγRs correlates with targeted cytotoxicity in cell-based assays (Shields et al., 2001, J Biol Chem 276:6591-6604; Presta et al., 2002, Biochem Soc Trans 30:487-490; Shields et al., 2002, J Biol Chem 277:26733-26740). Additionally, a correlation has been observed between clinical efficacy in humans and their allotype of high (V158) or low (F158) affinity polymorphic forms of FcγRIIIa (Cartron et al., 2002, Blood 99:754-758)(Weng & Levy, 2003, Journal of Clinical Oncology, 21:3940-3947). Together these data suggest that an antibody that is optimized for binding to certain FcγRs may better mediate effector functions and thereby destroy cancer cells more effectively in patients. The balance between activating and inhibiting receptors is an important consideration, and optimal effector function may result from an antibody that has enhanced affinity for activation receptors, for example FcγRI, FcγRIIa/c, and FcγRIIIa, yet reduced affinity for the inhibitory receptor FcγRIIb. Furthermore, because FcγRs can mediate antigen uptake and processing by antigen presenting cells, enhanced FcγR affinity may also improve the capacity of antibody therapeutics to elicit an adaptive immune response. With respect to EGFR, ADCC has been implicated as an important effector mechanism for the anti-tumor cytotoxic capacity of some anti-EGFR antibodies (Bleeker et al., 2004, J Immunol. 173(7):4699-707; Bier et al., 1998, Cancer Immunol Immunother 46:167-173).

Mutagenesis studies have been carried out on Fc towards various goals, with substitutions typically made to alanine (referred to as alanine scanning) or guided by sequence homology substitutions (Duncan et al., 1988, Nature 332:563-564; Lund et al., 1991, J Immunol 147:2657-2662; Lund et al., 1992, Mol Immunol 29:53-59; Jefferis et al., 1995, Immunol Lett 44:111-117; Lund et al., 1995, Faseb J 9:115-119; Jefferis et al., 1996, Immunol Lett 54:101-104; Lund et al., 1996, J Immunol 157:4963-4969; Armour et al., 1999, Eur J Immunol 29:2613-2624; Shields et al., 2001, J Biol Chem 276:6591-6604; Jefferis et al., 2002, Immunol Lett 82:57-65) (U.S. Pat. No. 5,624,821; U.S. Pat. No. 5,885,573; PCT WO 00/42072; PCT WO 99/58572). The majority of substitutions reduce or ablate binding with FcγRs. However some success has been achieved at obtaining Fc variants with selectively enhanced binding to FcγRs, and in some cases these Fc variants have been shown to provide enhanced potency and efficacy in cell-based effector function assays. See for example U.S. Pat. No. 5,624,821, PCT WO 00/42072, U.S. Pat. No. 6,737,056, U.S. Ser. No. 10/672,280, PCT U.S. Pat. No. 03/30249, and U.S. Ser. No. 10/822,231, and U.S. Ser. No. 60/627,774, filed Nov. 12, 2004 and entitled “Optimized Fc Variants”, and references cited therein. Enhanced affinity of Fc for FcγR has also been achieved using engineered glycoforms generated by expression of antibodies in engineered or variant cell lines (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473).

The present invention provides variants of EGFR targeting proteins that comprise one or more amino acid modifications that provide enhanced effector function. A variety of modifications are described that provide EGFR targeting proteins with optimized clinical properties. A broad array of applications of the EGFR targeting proteins are contemplated.

### SUMMARY OF THE INVENTION

The present invention provides variant EGFR targeting proteins that are optimized for a number of therapeutically relevant properties. A variant EGFR targeting protein comprises one or more amino acid modifications relative to a parent EGFR targeting protein, wherein said amino acid modification(s) provide one or more optimized properties. Suitable positions for the amino acid modifications include one or more of the following positions 230, 240, 244, 245, 247, 262, 263, 266, 273, 275, 299, 302, 313, 323, 325, 328, and 332.

For example, in some embodiments, variant proteins comprising an immunoglobulin constant chain, and amino acid modification selected from the group consisting of: P230A, E233D, L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V2401, V240A, V240T, V240M, F241W, F241L, F241Y, F241E, F241R, F243W, F243L F243Y, F243R, F243Q, P244H, P245A, P247V, P247G, V262I, V262A, V262T, V262E, V2631, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, S267T, S267H, S267D, S267N, E269H, E269Y, E269F, E269R, E269T, E269L, E269N, D270Q, D270T, D270H, E272S, E272K, E272I, E272Y, V273I, K274T, K274E, K274R, K274L, K274Y, F275W, N276S, N276E, N276R, N276L, N276Y, Y278T, Y278E, Y278K, Y278W, E283R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, V302I, W313F, E318R, K320T, K320D, K320I, K322T, K322H, V323I, S324T, S324D, S324R, S3241, S324V, S324L, S324Y, N325Q, N325L, N325I, N325D, N325E, N325A, N325T, N325V, N325H, K326L, K326I, K326T, A327N, A327L, A327D, A327T, L328M, L328D, L328E, L328N, L328Q, L328F, L328I, L328V, L328T, L328H, L328A, P329F, A330L, A330Y, A330V, A330I, A330F, A330R, A330H, A330S, A330W, A330M, P331V, P331H, I332D, I332E, I332N, 1332Q, I332T, I332H, I332Y, I332A, E333T, E333H, E333I, E333Y, K3341, K334T, K334F, T335D, T335R, T335Y, D221K, D221Y, K222E, K222Y, T223E, T223K, H224E, H224Y, T225E, T225E, T225K, T225W, P227E, P227K, P227Y, P227G, P228E, P228K, P228Y, P228G, P230E, P230Y, P230G, A231E, A231K, A231Y, A231P, A231G, P232E, P232K, P232Y, P232G, E233N, E233Q, E233K, E233R, E233S, E233T, E233H, E233A, E233V, E233L, E2331, E233F, E233M, E233Y, E233W, E233G, L234K, L234R, L234S, L234A, L234M, L234W, L234P, L234G, L235E, L235K, L235R, L235A, L235M, L235W, L235P, L235G, G236D, G236E, G236N, G236Q, G236K, G236R, G236S, G236T, G236H, G236A, G236V, G236L, G236I, G236F, G236M, G236Y, G236W, G236P, G237D, G237E, G237N, G237Q, G237K, G237R, G237S, G237T, G237H, G237V, G237L, G237I, G237F, G237M, G237Y, G237W, G237P, P238D, P238E, P238N, P238Q, P238K, P238R, P238S, P238T, P238H, P238V, P238L, P238I, P238F, P238M, P238Y, P238W, P238G, S239Q, S239K, S239R, S239V, S239L, S239I, S239M, S239W, S239P, S239G, F241D, F241E, F241Y, F243E, K246D, K246E, K246H, K246Y, D249Q, D249H, D249Y, R255E, R255Y, E258S, E258H, E258Y, T260D, T260E, T260H, T260Y, V262E, V262F, V264D, V264E, V264N, V264Q, V264K, V264R, V264S, V264H, V264W, V264P, V264G, D265Q, D265K, D265R, D265S, D265T, D265H, D265V, D265L, D265I, D265F, D265M, D265Y, D265W, D265P, S267E, S267Q, S267K, S267R, S267V, S267L, S267I, S267F, S267M, S267Y, S267W, S267P, H268D, H268E, H268Q, H268K, H268R, H268T, H268V, H268L, H268I, H268F, H268M, H268W, H268P, H268G, E269K, E269S, E269V, E2691, E269M, E269W, E269P, E269G, D270R, D270S, D270L, D270I, D270F, D270M, D270Y, D270W, D270P, D270G, P271D, P271E, P271N, P271Q, P271K, P271R, P271S, P271T, P271H, P271A, P271V, P271L, P271I, P271F, P271M, P271Y, P271W, P271G, E272D, E272R, E272T, E272H, E272V, E272L, E272F, E272M, E272W, E272P, E272G, K274D, K274N, K274S, K274H, K274V, K274I, K274F, K274M, K274W, K274P, K274G, F275L, N276D, N276T, N276H, N276V, N276I, N276F, N276M, N276W, N276P, N276G, Y278D, Y278N, Y278Q, Y278R, Y278S, Y278H, Y278V, Y278L, Y2781, Y278M, Y278P, Y278G, D280K, D280L, D280W, D280P, D280G, G281D, G281K, G281Y, G281P, V282E, V282K, V282Y, V282P, V282G, E283K, E283H, E283L, E283Y, E283P, E283G, V284E, V284N, V284T, V284L, V284Y, H285D, H285E, H285Q, H285K, H285Y, H285W, N286E, N286Y, N286P, N286G, K288D, K288E, K288Y, K290D, K290N, K290H, K290L, K290W, P291D, P291E, P291Q, P291T, P291H, P291I, P291G, R292D, R292E, R292T, R292Y, E293N, E293R, E293S, E293T, E293H, E293V, E293L, E293I, E293F, E293M, E293Y, E293W, E293P, E293G, E294K, E294R, E294S, E294T, E294H, E294V, E294L, E294I, E294F, E294M, E294Y, E294W, E294P, E294G, Q295D, Q295E, Q295N, Q295R, Q295S, Q295T, Q295H, Q295V, Q295I, Q295F, Q295M, Q295Y, Q295W, Q295P, Q295G, Y296K, Y296R, Y296A, Y296V, Y296M, Y296G, N297Q, N297K, N297R, N297T, N297H, N297V, N297L, N297I, N297F, N297M, N297Y, N297W, N297P, N297G, S298D, S298E, S298Q, S298K, S298R, S298I, S298F, S298M, S298Y, S298W, T299D, T299E, T299N, T299Q, T299K, T299R, T299L, T299F, T299M, T299Y, T299W, T299P, T299G, Y300D, Y300E, Y300N, Y300Q, Y300K, Y300R, Y300S, Y300T, Y300H, Y300A, Y300V, Y300M, Y300W, Y300P, Y300G, R301D, R301E, R301H, R301Y, V303D, V303E, V303Y, S304D, S304N, S304T, S304H, S304L, V305E, V305T, V305Y, K317E, K317Q, E318Q, E318H, E318L, E318Y, K320N, K320S, K320H, K320V, K320L, K320F, K320Y, K320W, K320P, K320G, K322D, K322S, K322V, K322I, K322F, K322Y, K322W, K322P, K322G, S324H, S324F, S324M, S324W, S324P, S324G, N325K, N325R, N325S, N325F, N325M, N325Y, N325W, N325P, N325G, K326P, A327E, A327K, A327R, A327H, A327V, A327I, A327F, A327M, A327Y, A327W, A327P, L328D, L328Q, L328K, L328R, L328S, L328T, L328V, L3281, L328Y, L328W, L328P, L328G, P329D, P329E, P329N, P329Q, P329K, P329R, P329S, P329T, P329H, P329V, P329L, P329I, P329M, P329Y, P329W, P329G, A330E, A330N, A330T, A330P, A330G, P331D, P331Q, P331R, P331T, P331L, P331I, P331F, P331M, P331Y, P331W, I332K, I332R, I332S, I332V, I332F, I332M, I332W, I332P, I332G, E333L, E333F, E333M, E333P, K334P, T335N, T335S, T335H, T335V, T335L, T335I, T335F, T335M, T335W, T335P, T335G, I336E, I336K, 1336Y, S337E, S337N, and S337H, are provided using the methods described herein (wherein numbering is according to the EU index as in Kabat). One or more additional substitutions can be selected from the group consisting of S298A, K326A, K326S, K326N, K326Q, K326D, K325E, K326W, K326Y, E333A, E333S, K334A, K334E, Y300I, Y300L, Q295K, E294N, S298N, S298V, S298D, D280H, K290S, D280Q, D280Y, K290G, K290T, K290Y, T250Q, T250E, M428L, and M428F.

In other embodiments, variant proteins comprising an immunoglobulin constant chain and amino acid modifications selected from the group consisting of S239D, S239E, S239N, S239Q, S239T, V240I, V240M, V2641, V264T, V264Y, E272Y, K274E, Y278T, 297D, T299A, T299V, T299I, T299H, K326T, L328A, L328H, A330Y, A330L, A330I, I332D, I1332E, 1332N, and 1332Q are provided herein.

In yet other embodiments, variant proteins comprising an immunoglobulin constant chain and amino acid modifications selected from the group consisting of I332E, V264I/I332E, S239D/I332E, or S239D/A330/I332E are provided herein.

It is an object of the present invention to provide variant EGFR targeting proteins that bind with greater affinity to one or more FcγRs relative to the parent protein. In a preferred embodiment, said FcγR is human FcγRIII.

It is an object of the present invention to provide variant EGFR targeting proteins that bind with reduced affinity to one or more FcγRs relative to the parent protein. In a preferred embodiment, said FcγR is human FcγRIIb.

It is a further object of the present invention to provide variant EGFR targeting proteins that mediate effector function more effectively in the presence of effector cells relative to the parent EGFR targeting protein. In a preferred embodiment, said variants mediate ADCC that is greater than that mediated by the parent. In an alternately preferred embodiment, said variants mediate ADCP that is greater than that mediated by the parent. In an alternate embodiment, said variants mediate CDC that is greater than that mediated by the parent.

It is a further object of the present invention to provide variant EGFR targeting proteins that have reduced immunogenicity relative to the parent protein.

The present invention also provides methods for engineering EGFR targeting proteins.

The present invention provides isolated nucleic acids encoding the EGFR targeting proteins described herein. The present invention provides vectors comprising said nucleic acids, optionally, operably linked to control sequences. The present invention provides host cells containing the vectors, and methods for producing and optionally recovering the variant EGFR targeting proteins.

The present invention provides novel antibodies and Fc fusions that target EGFR. Said novel antibodies and Fc fusions may find use in a therapeutic product.

The present invention provides compositions comprising the EGFR targeting proteins described herein, and a physiologically or pharmaceutically acceptable carrier or diluent.

The present invention contemplates therapeutic and diagnostic uses for the EGFR targeting proteins disclosed herein.

### BRIEF DESCRIPTION OF THE DRAWINGS

FIG. 1. The amino acid sequence of the heavy chain of the human IgG1 constant region. Positions are numbered according to the EU index as in Kabat below the amino acid sequence. The approximate beginnings of CH1 domain, hinge, CH2 domain, and CH3 domain are labeled above the sequence. Polymorphisms have been observed at a number of Fc positions, including but not limited to 270, 272, 312, 315, 356, and 358(Kim et al., 2001, J. Mol. Evol. 53:1-9) and thus slight differences between the presented sequence and sequences in the prior art may exist. Bolded residues indicate residues that are mutated in Example 1 to provide enhanced effector function, including S239, V264, A330, and I332.

FIG. 2. Amino acid sequences of the WT C225 VL (FIG. 2a) and VH (FIG. 2b) regions.

FIG. 3. Binding to human V158 FcγRIIIa by C225 WT and variant (V264I/I332E, S239D/I332E, and S239D/A330L/I332E) antibodies as determined by the AlphaScreen™ assay. In the presence of competitor antibody (Fc variant or WT C225) a characteristic inhibition curve is observed as a decrease in luminescence signal. Phosphate buffer saline (PBS) alone was used as the negative control. The binding data were normalized to the maximum and minimum luminescence signal for each particular curve, provided by the baselines at low and high antibody concentrations respectively. The curves represent the fits of the data to a one site competition model using nonlinear regression. These fits provide IC50s for each antibody, illustrated for WT and S239D by the dotted lines.

FIGS. 4. Cell-based ADCC assay of C225 Fc variants. Purified human peripheral blood monocytes (PBMCs) were used as effector cells, A431 epidermoid carcinoma cells were used as target cells at a 10:1 effector:target cell ratio, and lysis was monitored by measuring LDH activity using the Cytotoxicity Detection Kit (LDH, Roche Diagnostic Corporation, Indianapolis, Ind.). Samples were run in triplicate to provide error estimates (n=3, ±S.D.). FIG. 4 shows the dose dependence of ADCC at various antibody concentrations, normalized to the minimum and maximum levels of lysis for the assay. The curves represent the fits of the data to a sigmoidal dose-response model using nonlinear regression.

FIG. 5. The amino acid sequence of the heavy chain of the human IgG2 constant region. Positions are numbered according to the EU index as in Kabat below the amino acid sequence. The approximate beginnings of CH1 domain, hinge, CH2 domain, and CH3 domain are labeled above the sequence. Polymorphisms have been observed at a number of Fc positions (Kim et al., 2001, J. Mol. Evol. 53:1-9) and thus slight differences between the presented sequence and sequences in the prior art may exist. Bolded residues indicate residue that are mutated in Example 1 to provide enhanced effector function, including P233, V234, A235, -236, S239, V264, G327, A330, and 1332, where −236 indicates the absence of an amino acid (a deletion) at EU index position 236.

FIG. 6. Amino acid sequences of the WT ICR62 VL (FIG. 5a) and VH (FIG. 5b) regions.

FIG. 7. Sequences of C225 VL and VH region variants with reduced immunogenicity. Differences between the variants and WT C225 are bolded.

FIG. 8. Sequences of ICR62 VL and VH region variants with reduced immunogenicity. Differences between the variants and WT ICR62 are bolded.

FIG. 9. Surface Plasmon Resonance (SPR) (Biacore, Uppsala, Sweden) sensorgrams showing binding of C225 variants to the EGFR target antigen. The sensorgrams show the binding of L2/H3 and L2/H4 C225 variant Fabs to an EGFR coupled sensor chip surface.

FIG. 10. SPR sensorgrams showing binding of ICR62 variant Fabs to the EGFR target antigen. The sensorgrams show the binding of WT and L2/H9 ICR62 variant Fabs to an EGFR coupled sensor chip surface at varying concentrations of antibody.

FIG. 11. SPR sensorgrams showing binding of full length antibody C225 variants to the EGFR target antigen. The sensorgrams show binding of C225 WT (L0/H0) and variant (L0/H3, L0/H4, L0/H5, L0/H6, L0/H7, L0/H8, L2/H3, L2/H4, L2/H5, L2/H6, L2/H7, L2/H8, L3/H3, L3/H4, L3/H5, L3/H6, L3/H7, L3/H8, L4/H3, L4/H4, L4/H5, L4/H6, L4/H7, and L4/H8) full length antibodies to the EGFR sensor chip. The curves consist of a association phase and dissociation phase, the separation being marked by a little spike on each curve.

FIGS. 12. Cell-based ADCC assay of C225 WT (L0/H0) and variant (L0/H3, L0/H4, L0/H5, L0/H6, L0/H7, L0/H8, L2/H3, L2/H4, L2/H5, L2/H6, L2/H7, L2/H8, L3/H3, L3/H4, L3/H5, L3/H6, L3/H7, L3/H8, L4/H3, L4/H4, L4/H5, L4/H6, L4/H7, and L4/H8) full length antibodies. Purified human peripheral blood monocytes (PBMCs) were used as effector cells, A431 epidermoid carcinoma cells were used as target cells at a 10:1 effector:target cell ratio, and lysis was monitored by measuring LDH activity using the Cytotoxicity Detection Kit (LDH, Roche Diagnostic Corporation, Indianapolis, Ind.). Samples were run in triplicate to provide error estimates (n=3, ±S.D.). FIG. 12 shows the dose dependence of ADCC at various antibody concentrations, normalized to the minimum and maximum levels of lysis for the assay. The curves represent the fits of the data to a sigmoidal dose-response model using nonlinear regression.

FIG. 13. Amino acid sequences of an EGFR targeting IgG1 antibody comprising the L3 C225 variant VL with the CLκ constant light chain (FIG. 13a), the H4 C225 variant VH with an IgG1 constant chain that may comprise amino acid modifications in the Fc region (FIG. 13b). Positions that may be mutated as described are designated in bold as X₁, X₂, X₃, and X₄, referring to residues S239, V264, A330, and 1332. FIG. 13c provides one example of a heavy chain described in FIG. 13b, here comprising the H4 C225 variant VH region with the S239D/A330L/I332E IgG1 constant region.

FIG. 14. Amino acid sequences of an EGFR targeting IgG2 antibody comprising the L4 C225 variant VL with the CLκ constant light chain (FIG. 14a) , the H7 C225 variant VH with an IgG2 constant chain that may comprise amino acid modifications in the Fc region (FIG. 14b). Positions that may be mutated as described are designated in bold as X₁, X₂, X₃, X₄, Z₁, Z₂, Z₃, Z₄, and Z₅ referring to residues S239, V264, A330, I332, P233, V234, A235, −236, and G237 (here −236 refers to a deletion at EU index position 236). FIG. 14c provides one example of a heavy chain described in FIG. 14b, here comprising the H7 C225 variant VH region with the S239D/A330L/I332E/P233EN234L/A235L/−236G IgG2 constant region.

### DETAILED DESCRIPTION OF THE INVENTION

In order that the invention may be more completely understood, several definitions are set forth below. Such definitions are meant to encompass grammatical equivalents.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “amino acid modification” herein is meant an amino acid substitution, insertion, and/or deletion in a polypeptide sequence. The preferred amino acid modification herein is a substitution. By “amino acid substitution” or “substitution” herein is meant the replacement of an amino acid at a particular position in a parent polypeptide sequence with another amino acid. For example, the substitution 1332E refers to a variant polypeptide, in this case an Fc variant, in which the isoleucine at position 332 is replaced with a glutamic acid.

By “antibody” herein is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes respectively. Antibody herein is meant to include full length antibodies and antibody fragments, and may refer to a natural antibody from any organism, an engineered antibody, or an antibody generated recombinantly for experimental, therapeutic, or other purposes as further defined below. The term “antibody” includes antibody fragments, as are known in the art, such as Fab, Fab′, F(ab′)₂, Fv, scFv, or other antigen-binding subsequences of antibodies, either produced by the modification of whole antibodies or those synthesized de novo using recombinant DNA technologies. Particularly preferred are full length antibodies that comprise Fc variants as described herein. The term “antibody” comprises monoclonal and polyclonal antibodies. Antibodies can be antagonists, agonists, neutralizing, inhibitory, or stimulatory.

Specifically included within the definition of “antibody” are full-length antibodies that contain an Fc variant portion. By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In some mammals, for example in camels and llamas, IgG antibodies may consist of only two heavy chains, each heavy chain comprising a variable domain attached to the Fc region. By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3.

By “amino acid” and “amino acid identity” as used herein is meant one of the 20 naturally occurring amino acids or any non-natural analogues that may be present at a specific, defined position. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e. “analogs”, such as peptoids (see Simon et al., 1992, Proc Natl Acad Sci USA 89(20):9367) particularly when LC peptides are to be administered to a patient. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

By “effector function” as used herein is meant a biochemical event that results from the interaction of an antibody Fc region with an Fc receptor or ligand. Effector functions include but are not limited to ADCC, ADCP, and CDC. By “effector cell” as used herein is meant a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and γγ T cells, and may be from any organism including but not limited to humans, mice, rats, rabbits, and monkeys. By “library” herein is meant a set of Fc variants in any form, including but not limited to a list of nucleic acid or amino acid sequences, a list of nucleic acid or amino acid substitutions at variable positions, a physical library comprising nucleic acids that encode the library sequences, or a physical library comprising the Fc variant proteins, either in purified or unpurified form.

By “EGFR targeting protein” as used herein is meant a protein that binds to the epidermal growth factor receptor (EGFR, ErbB-1, HER1). The EGFR targeting protein of the present invention may be an antibody, Fc fusion, or any other protein that binds EGFR. An EGFR targeting protein of the present invention may bind any epitope or region on EGFR, and may be specific for fragments, splice forms, or aberrant forms of EGFR.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cgamma2 and Cgamma3 (Cγ2 and Cγ3) and the hinge between Cgamma1 (Cγ1) and Cgamma2 (Cγ2). Although the boundaries of the Fc region may vary, the human IgG heavy chain Fc region is usually defined to comprise residues C226 or P230 to its carboxyl-terminus, wherein the numbering is according to the EU index as in Kabat. Fc may refer to this region in isolation, or this region in the context of an Fc polypeptide, as described below. By “Fc Polypeptide” as used herein is meant a polypeptide that comprises all or part of an Fc region. Fc polypeptides include antibodies, Fc fusions, isolated Fcs, and Fc fragments.

By “Fc fusion” as used herein is meant a protein wherein one or more polypeptides or small molecules is operably linked to an Fc region or a derivative thereof. Fc fusion is herein meant to be synonymous with the terms “immunoadhesin”, “Ig fusion”, “Ig chimera”, and “receptor globulin” (sometimes with dashes) as used in the prior art (Chamow et al., 1996, Trends Biotechnol 14:52-60; Ashkenazi et al., 1997, Curr Opin Immunol 9:195-200). An Fc fusion combines the Fc region of an immunoglobulin with a fusion partner, which in general can be any protein or small molecule. The role of the non-Fc part of an Fc fusion, i.e. the fusion partner, is often but not always to mediate target binding, and thus it is functionally analogous to the variable regions of an antibody. A variety of linkers, defined and described below, may be used to covalently link Fc to a fusion partner to generate an Fc fusion.

By “Fc gamma receptor” or “FcγR” as used herein is meant any member of the family of proteins that bind the IgG antibody Fc region and are substantially encoded by the FcγR genes. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIIb-NA1 and FcγRIIIb-NA2) (Jefferis et al., 2002, Immunol Lett 82:57-65), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “Fc ligand” as used herein is meant a molecule, preferably a polypeptide, from any organism that binds to the Fc region of an antibody to form an Fc-ligand complex. Fc ligands include but are not limited to FcγRs, FcγRs, FcγRs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136). Fc ligands may include undiscovered molecules that bind Fc.

By “IgG” as used herein is meant a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full length antibodies, antibody fragments, and individual immunoglobulin domains. By “immunoglobulin (Ig) domain” herein is meant a region of an immunoglobulin that exists as a distinct structural entity as ascertained by one skilled in the art of protein structure. Ig domains typically have a characteristic β-sandwich folding topology. The known Ig domains in the IgG class of antibodies are V_{H}, Cγ1, Cγ2, Cγ3, V_{L}, and C_{L}.

By “parent polypeptide” or “precursor polypeptide” (including Fc parent or precursors) as used herein is meant a polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant a Fc polypeptide that is modified to generate a variant, and by “parent antibody” as used herein is meant an antibody that is modified to generate a variant antibody.

As outlined above, certain positions of the Fc molecule can be altered. By “position” as used herein is meant a location in the sequence of a protein. Positions may be numbered sequentially, or according to an established format, for example the EU index as in Kabat. For example, position 297 is a position in the human antibody IgG1. Corresponding positions are determined as outlined above, generally through alignment with other parent sequences.

By “residue” as used herein is meant a position in a protein and its associated amino acid identity. For example, Asparagine 297 (also referred to as Asn297, also referred to as N297) is a residue in the human antibody IgG1.

By “target antigen” as used herein is meant the molecule that is bound specifically by the variable region of a given antibody. A target antigen may be a protein, carbohydrate, lipid, or other chemical compound.

By “target cell” as used herein is meant a cell that expresses a target antigen.

By “variable region” as used herein is meant the region of an immunoglobulin that comprises one or more Ig domains substantially encoded by any of the Vκ, Vλ, and/or V_{H} genes that make up the kappa, lambda, and heavy chain immunoglobulin genetic loci respectively.

By “variant protein”, “protein variant”, “variant polypeptide”, or “polypeptide variant” as used herein is meant a polypeptide sequence that differs from that of a parent polypeptide sequence by virtue of at least one amino acid modification. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% homology with a parent polypeptide sequence, and most preferably at least about 90% homology, more preferably at least about 95% homology. Accordingly, by “variant Fc” or “Fc variant” as used herein is meant an Fc sequence that differs from that of a parent Fc sequence by virtue of at least one amino acid modification. An Fc variant may only encompass an Fc region, or may exist in the context of an antibody, Fc fusion, or other polypeptide that is substantially encoded by Fc. Fc variant may refer to the Fc polypeptide itself, compositions comprising the Fc variant polypeptide, or the amino acid sequence that encodes it. Accordingly, by “variant EGFR targeting protein” or “EGFR targeting protein variant” as used herein is meant an EGFR targeting protein, as defined above, that differs in sequence from that of a parent EGFR targeting protein sequence by virtue of at least one amino acid modification. Variant EGFR targeting protein may refer to the protein itself, compositions comprising the protein, or the amino acid sequence that encodes it.

For all immunoglobulin heavy chain constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda). The “EU index as in Kabat” refers to the residue numbering of the human IgG1 EU antibody.

EGFR Targeting Proteins

The EGFR targeting proteins of the present invention may be an antibody, referred to herein as “anti-EGFR antibodies”. Anti-EGFR antibodies of the present invention may comprise immunoglobulin sequences that are substantially encoded by immunoglobulin genes belonging to any of the antibody classes, including but not limited to IgG (including human subclasses IgG1, IgG2, IgG3, or IgG4), IgA (including human subclasses IgA1 and IgA2), IgD, IgE, IgG, and IgM classes of antibodies. Most preferably the antibodies of the present invention comprise sequences belonging to the human IgG class of antibodies. Anti-EGFR antibodies of the present invention may be nonhuman, chimeric, humanized, or fully human. As will be appreciated by one skilled in the art, these different types of antibodies reflect the degree of “humanness” or potential level of immunogenicity in a human. For a description of these concepts, see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). Said nonhuman variable region may be derived from any organism as described above, preferably mammals and most preferably rodents or primates. In one embodiment, the antibody of the present invention comprises monkey variable domains, for example as described in Newman et al., 1992, Biotechnology 10:1455-1460, U.S. Pat. No. 5,658,570, and U.S. Pat. No. 5,750,105. In a preferred embodiment, the variable region is derived from a nonhuman source, but its immunogenicity has been reduced using protein engineering. In a preferred embodiment, the antibodies of the present invention are humanized (Tsurushita & Vasquez, 2004, Humanization of Monoclonal Antibodies, Molecular Biology of B Cells, 533-545, Elsevier Science (USA)). By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (see, for example, Winter U.S. Pat. No. 5,225,539). This strategy is referred to as “CDR grafting”. “Backmutation” of selected acceptor framework residues to the corresponding donor residues is often required to regain affinity that is lost in the initial grafted construct (see, for example, U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. In a most preferred embodiment, and as described more fully in Example 2 infra, the immunogenicity of the antibody has been reduced using a method described in U.S. Ser. No. 60/619,483, filed Oct. 14, 2004 and U.S. Ser. No. 10/______, entitled “Methods of Generating Variant Proteins with Increased Host String Content and Compositions Thereof”, filed on Dec. 6, 2004. In an alternate embodiment, the antibodies of the present invention may be fully human, that is the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

The variable regions of any known or undiscovered anti-EGFR antibody may find use in the present invention. A number of useful antibodies have been discovered that target EGFR, including, but not limited to, Cetuximab (Erbitux®, Imclone) (U.S. Pat. No. 4,943,533; PCT WO 96/40210); ABX-EGF (Abgenix-Immunex-Amgen) (U.S. Pat. No. 6,235,883; Yang et al., 2001, Crit. Rev. Oncol. Hematol. 38:17-23); HuMax-EGFr (Genmab) (U.S. Ser. No. 10/172,317), 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); ICR62 (Institute of Cancer Research) (PCT WO 95/20045; Modjtahedi et al., 1993, J. Cell Biophys. 1993, 22(1-3):129-46; Modjtahedi et al., 1993, Br J Cancer); hR3 (U.S. Pat. No. 5,891,996; Mateo et al., 1997, Immunotechnology 3(1):71-81; mAb-806 (Jngbluth et al. 2003, Proc Natl Acad Sci U S A. 100(2):639-44; MR1-1 (WO 01/62931); SC100 (WO 01/88138); MDX-447, H22-EGF, 528(IgG1).

The EGFR targeting proteins of the present invention may be an Fc fusion, referred to herein as “anti-EGFR Fc fusions”. Anti-EGFR Fc fusions of the present invention comprise an Fc polypeptide operably linked to one or more fusion partners. The role of the fusion partner typically, but not always, is to mediate binding of the Fc fusion to a target antigen. (Chamow et al., 1996, Trends Biotechnol 14:52-60; Ashkenazi et al., 1997, Curr Opin Immunol 9:195-200). For the present invention, one of the fusion partners must bind EGFR. Fusion partners may be a protein, polypeptide, or small molecule. Virtually any polypeptide or molecule that targets EGFR may serve as a fusion partner, including but not limited to epidermal growth factor (EGF), transforming growth factor-α (TGFα), amphiregulin, heparin-binding EGF-like growth factor, betacellulin, epiregulin, CRIPTO (teratocarcinoma-derived growth factor), and vaccinnia virus growth factor (Salomon et al., 1995, Crit. Rev. Oncol. Hematol. 19:183-232). Undiscovered EGFR ligands may serve as fusion partners for the EGFR targeting proteins of the present invention. Variants of the EGFR ligands may also be used in the present invention. In one example, an EGFR ligand may be engineered to not agonize or alternatively antagonize, EGFR. Anti-EGFR Fc fusions of the invention may comprise immunoglobulin sequences that are substantially encoded by immunoglobulin genes belonging to any of the antibody classes, including but not limited to IgG (including human subclasses IgG1, IgG2, IgG3, or IgG4), IgA (including human subclasses IgA1 and IgA2), IgD, IgE, IgG, and IgM classes of antibodies. Most preferably the anti-EGFR Fc fusions of the present invention comprise sequences belonging to the human IgG class of antibodies.

EGFR targeting proteins of the present invention, including antibodies and Fc fusions, may comprise Fc fragments. An Fc fragment of the present invention may comprise from about 1-90% of the Fc region, with about 10-90% being preferred, and about 30-90% being most preferred. Thus for example, an Fc fragment of the present invention may comprise an IgG1 Cγ2 domain, an IgG1 Cγ2 domain and hinge region, an IgG1 Cγ3 domain, and so forth. In one embodiment, an Fc fragment of the present invention additionally comprises a fusion partner, effectively making it an Fc fragment fusion. Fc fragments may or may not contain extra polypeptide sequences.

EGFR targeting proteins of the present invention may be substantially encoded by genes from any organism, preferably mammals, including but not limited to humans, rodents including but not limited to mice and rats, lagomorpha including but not limited to rabbits and hares, camelidae including but not limited to camels, llamas, and dromedaries, and non-human primates, including but not limited to Prosimians, Platyrrhini (New World monkeys), Cercopithecoidea (Old World monkeys), and Hominoidea including the Gibbons and Lesser and Great Apes. In a most preferred embodiment, the EGFR targeting proteins of the present invention are substantially human. The EGFR targeting proteins of the present invention may be substantially encoded by immunoglobulin genes belonging to any of the antibody classes. In a most preferred embodiment, the EGFR targeting proteins of the present invention comprise sequences belonging to the IgG class of antibodies, including human subclasses IgG1, IgG2, IgG3, and IgG4. In an alternate embodiment, the EGFR targeting proteins of the present invention comprise sequences belonging to the IgA (including human subclasses IgA1 and IgA2), IgD, IgE, IgG, or IgM classes of antibodies. The EGFR targeting proteins of the present invention may comprise more than one protein chain. That is, the present invention may find use in an EGFR targeting protein that is a monomer or an oligomer, including a homo- or hetero-oligomer.

In the most preferred embodiment, the anti-EGFR antibodies and Fc fusions of the invention are based on human IgG sequences, and thus human IgG sequences are used as the “base” sequences against which other sequences are compared, including but not limited to sequences from other organisms, for example rodent and primate sequences, as well as sequences from other immunoglobulin classes such as IgA, IgE, IgGD, IgGM, and the like. It is contemplated that, although the EGFR targeting proteins of the present invention are engineered in the context of one parent EGFR targeting protein, the variants may be engineered in or “transferred” to the context of another, second parent EGFR targeting protein. This is done by determining the “equivalent” or “corresponding” residues and substitutions between the first and second EGFR targeting proteins, typically based on sequence or structural homology between the sequences of the two EGFR targeting proteins. In order to establish homology, the amino acid sequence of a first EGFR targeting protein outlined herein is directly compared to the sequence of a second EGFR targeting protein. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first EGFR targeting protein are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second EGFR targeting protein that is at the level of tertiary structure for EGFR targeting proteins whose structures have been determined. In this case, equivalent residues are defined as those for which the atomic coordinates of two or more of the main chain atoms of a particular amino acid residue of the parent or precursor (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent EGFR targeting protein in which the EGFR targeting proteins are made, what is meant to be conveyed is that the EGFR targeting proteins discovered by the present invention may be engineered into any second parent EGFR targeting protein that has significant sequence or structural homology with said EGFR targeting protein. Thus, for example, if a variant anti-EGFR antibody may be generated where the parent anti-EGFR antibody is human IgG1, by using the methods described above or other methods for determining equivalent residues, said variant anti-EGFR antibody may be engineered in a human IgG2 parent anti-EGFR antibody, a human IgA parent anti-EGFR antibody, a mouse IgG2a or IgG2b parent anti-EGFR antibody, and the like. Again, as described above, the context of the parent EGFR targeting protein does not affect the ability to transfer the EGFR targeting proteins of the present invention to other parent EGFR targeting proteins. For example, the variant anti-EGFR antibodies that are engineered in a human IgG1 antibody that targets one EGFR epitope may be transferred into a human IgG2 antibody that targets a different EGFR epitope, into an Fc fusion that comprises a human IgG1 Fc region that targets yet a different EGFR epitope, and so forth.

The EGFR targeting protein of the present invention may be virtually any antibody, Fc fusion, or other protein that binds EGFR. EGFR targeting proteins of the invention may display selectivity for EGFR versus alternative targets, for example other RTKs, or selectivity for a specific form of the EGFR target versus alternative forms. Examples include full-length versus splice variants, cell-surface vs. soluble forms, selectivity for various polymorphic variants, or selectivity for specific conformational forms of a target. An EGFR targeting protein of the present invention may bind any epitope or region on EGFR, and may be specific for fragments, mutant forms, splice forms, or aberrant forms of EGFR. For example, the anti-EGFR antibody mAb-806 binds a truncated version of EGFR called delta2-7 EGFR (Jungbluth et al., 2003, Proc Natl Acad Sci USA. 100(2): 639-644. As another example, the anti-EGFR antibody MR1-1, binds a mutant form of EGFR called EGFRvIII, but not WT EGFR (Landry et al., 2001, J. Mol. Biol. 308, 883-893). These antibodies or their variable regions may find use in the present invention.

The EGFR targeting proteins of the present invention may find use in a wide range of products. In one embodiment the EGFR targeting protein of the invention is a therapeutic, a diagnostic, or a research reagent, preferably a therapeutic. Alternatively, the EGFR targeting protein of the present invention may be used for agricultural or industrial uses. An anti-EGFR antibody of the present invention may find use in an antibody composition that is monoclonal or polyclonal. The EGFR targeting proteins of the present invention may be agonists, antagonists, neutralizing, inhibitory, or stimulatory. In a preferred embodiment, the EGFR targeting proteins of the present invention are used to kill target cells that bear the EGFR target antigen, for example cancer cells. In an alternate embodiment, the EGFR targeting proteins of the present invention are used to block, antagonize, or agonize the EGFR target antigen. In an alternately preferred embodiment, the EGFR targeting proteins of the present invention are used to block, antagonize, or agonize the target antigen and kill the target cells that bear the target antigen.

Modifications

The present invention provides variant EGFR targeting proteins that are optimized for a number of therapeutically relevant properties. A variant EGFR targeting protein comprises one or more amino acid modifications relative to a parent EGFR targeting protein, wherein said amino acid modification(s) provide one or more optimized properties. Thus the EGFR targeting proteins of the present invention are variants EGFR targeting proteins. An EGFR targeting protein of the present invention differs in amino acid sequence from its parent EGFR targeting protein by virtue of at least one amino acid modification. Thus variant EGFR targeting proteins of the present invention have at least one amino acid modification compared to the parent. Alternatively, the variant EGFR targeting proteins of the present invention may have more than one amino acid modification as compared to the parent, for example from about one to fifty amino acid modifications, preferably from about one to ten amino acid modifications, and most preferably from about one to about five amino acid modifications, each as compared to the parent. Thus the sequences of the variant EGFR targeting proteins and those of the parent EGFR targeting proteins are substantially homologous. For example, the variant EGFR targeting protein sequences of the present invention will preferably possess at least about 80% homology with the parent EGFR targeting protein sequence, more preferably at least about 90% homology, and most preferably at least about 95% homology.

In a most preferred embodiment, the EGFR targeting proteins of the present invention comprise amino acid modifications that provide optimized effector function properties relative to the parent. Most preferred substitutions and optimized effector function properties are described in U.S. Ser. No. 10/672,280, PCT U.S. Pat. No. 03/30249, and U.S. Ser. No. 10/822,231, and U.S. Ser. No. 60/627,774, filed Nov. 12, 2004 and entitled “Optimized Fc Variants”.

Variant proteins that target Epidermal Growth Factor Receptor (EGFR) with at least one amino acid modification relative to a parent protein are an aspect of the present invention. These variant proteins modulate binding to an FcγR or modulate effector function as compared to a parent protein. It is preferred that the parent the variable region of C225 or ICR162. The variant proteins of the present invention may be in the form of an antibody or Fc fusion. In either embodiment, the Fc region may be an IgG1, IgG2, IgG3 or IgG4, and most preferably an IgG1 or IgG2.

Preferably, the variant protein comprises an immunoglobulin constant chain and the amino acid modification is a substitution at a position selected from the group consisting of: 230, 240, 244, 245, 247, 262, 263, 266, 273, 275, 299, 302, 313, 323, 325, 328, and 332, wherein numbering is according to the EU index as in Kabat.

Examples of more preferred amino acid modifications include but are not limited to at least one of: P230A, E233D, L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L F243Y, F243R, F243Q, P244H, P245A, P247V, P247G, V2621, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, S267T, S267H, S267D, S267N, E269H, E269Y, E269F, E269R, E269T, E269L, E269N, D270Q, D270T, D270H, E272S, E272K, E272I, E272Y, V273I, K274T, K274E, K274R, K274L, K274Y, F275W, N276S, N276E, N276R, N276L, N276Y, Y278T, Y278E, Y278K, Y278W, E283R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, V302I, W313F, E318R, K320T, K320D, K320I, K322T, K322H, V323I, S324T, S324D, S324R, S324I, S324V, S324L, S324Y, N325Q, N325L, N325I, N325D, N325E, N325A, N325T, N325V, N325H, K326L, K326I, L328I, L328V, L328T, L328H, L328A, P329F, A330L, A330Y, A330V, A330I, A330F, A330R, A330H, A330S, A330W, A330M, P331V, P331H, 1332D, 1332E, 1332N, 1332Q, I332T, I332H, I332Y, I332A, E333T, E333H, E333I, E333Y, K334I, K334T, K334F, T335R, T335Y, 239D, 239E, 239N, 239Q, 239T, 240I, 240M, 264I, 264T, 264Y, 297D, 330I, 330L, 330Y, 332D, 332E, 332N, 332Q, A231E, A231G, A231K, A231P, A231Y, A298H, A327D, A327E, A327F, A327H, A327I, A327K, A327L, A327M, A327N, A327P, A327R, A327T, A327T, A327V, A327W, A327Y, A330E, A330F, A330F, A330G, A330H, A330I, A330L, A330M, A330N, A330P, A330R, A330S, A330T, A330V, A330W, A330Y, A330Y, D221K, D221Y, D249H, D249Q, D249Y, D265F, D265G, D265H, D265I, D265K, D265L, D265M, D265N, D265P, D265Q, D265R, D265S, D265T, D265V, D265W, D265Y, D270F, D270G, D270H, D270I, D270L, D270M, D270P, D270Q, D270R, D270S, D270T, D270W, D270Y, D280G, D280H, D280K, D280L, D280P, D280Q, D280W, D280Y, E233A, E233D, E233F, E233G, E233H, E233I, E233K, E233L, E233M, E233N, E233Q, E233R, E233S, E233T, E233V, E233W, E233Y, E258H, E258S, E258Y, E269F, E269G, E269H, E269I, E269K, E269L, E269M, E269N, E269P, E269R, E269S, E269T, E269V, E269W, E269Y, E272D, E272F, E272G, E272H, E272I, E272K, E272L, E272M, E272P, E272R, E272S, E272T, E272V, E272W, E272Y, E283G, E283H, E283K, E283L, E283P, E283R, E283Y, E293F, E293G, E293H, E293I, E293L, E293M, E293N, E293P, E293R, E293S, E293T, E293V, E293W, E293Y, E294F, E294G, E294H, E2941, E294K, E294L, E294M, E294N, E294P, E294R, E294S, E294T, E294V, E294W, E294Y, E318H, E318L, E318Q, E318R, E318Y, E333A, E333F, E333H, E333I, E333L, E333M, E333P, E333S, E333T, E333Y, E333Y, F241D, F241E, F243E, F243L, F243Q, F243R, F243W, F243W, F243Y, F275L, F275W, F275W, G236A, G236D, G236E, G236F, G236H, G236I, G236K, G236L, G236M, G236N, G236P, G236Q, G236R, G236S, G236T, G236V, G236W, G236Y, G237D, G237E, G237F, G237H, G237I, G237K, G237L, G237M, G237N, G237P, G237Q, G237R, G237S, G237T, G237V, G237W, G237Y, G281D, G281K, G281P, G281Y, H224E, H224Y, H268D, H268E, H268F, H268G, H268I, H268K, H268L, H268M, H268P, H268Q, H268R, H268T, H268V, H268W, H285D, H285E, H285K, H285Q, H285W, H285Y, I332A, I332D, I332E, I332F, I332G, I332H, I332K, I332L, I332M, I332N, I332N, I332P, I332Q, I332R, I332T, I332V, I332W, I332Y, I332Y, I336E, 1336K, I336Y, K222E, K222Y, K246D, K246H, K246Y, K274D, K274E, K274F, K274G, K274H, K274I, K274L, K274M, K274N, K274P, K274R, K274S, K274T, K274V, K274W, K274Y, K288D, K288E, K288Y, K290D, K290G, K290H, K290L, K290N, K290S, K290T, K290W, K290Y, K317E, K317Q, K320D, K320F, K320G, K320H, K320I, K320L, K320N, K320P, K320S, K320T, K320T, K320V, K320W, K320Y, K322D, K322F, K322G, K322H, K322I, K322P, K322S, K322T, K322V, K322W, K322Y, K325E, K326A, K326D, K3261, K326I, K326L, K326N, K326P, K326Q, K326S, K326T, K326T, K326W, K326Y, K334A, K334E, K334F, K3341, K334P, K334T, L234A, L234D, L234E, L234F, L234G, L234H, L2341, L2341, L234K, L234M, L234N, L234P, L234Q, L234R, L234S, L234T, L234V, L234W, L234Y, L235A, L235D, L235E, L235F, L235F, L235G, L235H, L235I, L235K, L235M, L235N, L235P, L235Q, L235R, L235S, L235T, L235V, L235W, L235Y, L328A, L328D, L328E, L328F, L328G, L328H, L328I, L328K, L328M, L328N, L328P, L328Q, L328Q, L328Q/I332E, L328R, L328S, L328T, L328V, L328V/I332E, L328W, L328Y, M428F M428L, N276D, N276E, N276F, N276G, N276H, N276I, N276L, N276M, N276P, N276R, N276S, N276T, N276V, N276W, N276Y, N276Y, N286E, N286G, N286P, N286Y, N297D, N297E, N297F, N297G, N297H, N297I, N297K, N297L, N297M, N297P, N297Q, N297R, N297S, N297S, N297T, N297V, N297W, N297Y, N325A, N325D, N325E, N325F, N325G, N325H, N325I, N325K, N325L, N325M, N325P, N325Q, N325Q, N325R, N325S, N325T, N325V, N325W, N325Y, P227E, P227G, P227K, P227Y, P228E, P228G, P228K, P228Y, P230A, P230E, P230G, P230Y, P232E, P232G, P232K, P232Y, P238D, P238E, P238F, P238G, P238H, P238I, P238K, P238L, P238M, P238N, P238Q, P238R, P238S, P238T, P238V, P238W, P238Y, P244H, P245A, P247G, P247V, P271A, P271D, P271E, P271F, P271G, P271H, P271I, P271K, P271L, P271M, P271N, P271Q, P271R, P271S, P271T, P271V, P271W, P271Y, P291D, P291E, P291G, P291H, P291I, P291Q, P291T, P329D, P329E, P329F, P329F, P329G, P329H, P329I, P329K, P329L, P329M, P329N, P329Q, P329R, P329S, P329T, P329V, P329W, P329Y, P331D, P331F, P331H, P331H, P331I, P331L, P331M, P331Q, P331R, P331T, P331V, P331W, P331Y, Q295D, Q295E, Q295F, Q295G, Q295H, Q295I, Q295K, Q295M, Q295N, Q295P, Q295R, Q295S, Q295T, Q295V, Q295W, Q295Y, R255E, R255Y, R292D, R292E, R292T, R292Y, R301D, R301E, R301H, R301Y, S239D, S239P, S239Q, S239R, S239T, S239V, S239W, S239Y, S267D, S267E, S267F, S267H, S267I, S267K, S267L, S267L/A327S, S267M, S267N, S267P, S267Q, S267Q/A327S, S267R, S267T, S267V, S267W, S267Y, S298A, S298D, S298E, S298F, S2981, S298K, S298M, S298N, S298Q, S298R, S298V, S298W, S298Y, S304D, S304H, S304L, S304N, S304T, S324D, S324F, S324G, S324H, S324I, S324L, S324M, S324P, S324R, S324T, S324V, S324W, S324Y, S337E, S337H, S337N, T223E, T223K, T225, T225E, T225K, T225W, T250E, T250Q, T260D, T260E, T260H, T260Y, T299A, T299D, T299E, T299F, T299G, T299H, T299I, T299K, T299L, T299L, T299M, T299N, T299P, T299Q, T299R, T299S, T299V, T299W, T299Y, T335D, T335F, T335G, T335H, T335I, T335L, T335M, T335N, T335P, T335R, T335S, T335V, T335W, T335Y, V240A, V240I, V2401N266I, V240M, V240T, V262A, V262E, V262F, V262I, V262T, V263A, V263I, V263M, V263T, V264D, V264E, V264F, V264G, V264H, V264I, V264K, V264L, V264M, V264N, V264P, V264Q, V264R, V264S, V264T, V264W, V264Y, V266A, V266I, V266M, V266T, V266T, V273I, V273I, V282E, V282G, V282K, V282P, V282Y, V284E, V284L, V284N, V284T, V284Y, V302I, V302I, V303D, V303E, V303Y, V305E, V305T, V305Y, V323I, V323I, W313F, Y278D, Y278E, Y278G, Y278H, Y278I, Y278K, Y278L, Y278M, Y278N, Y278P, Y278Q, Y278R, Y278S, Y278T, Y278V, Y278W, Y296A, Y296D, Y296I, Y296K, Y296L, Y296M, Y296N, Y296Q, Y296R, Y296S, Y296T, Y296V, Y300A, Y300D, Y300E, Y300G, Y300H, Y300I, Y300K, Y300L, Y300M, Y300N, Y300P, Y300Q, Y300R, Y300S, Y300T, Y300V, and Y300W.

More preferably, the variant protein of the present invention has at least one amino acid modification selected from: S239D, S239E, S239N, S239Q, S239T, V240I, V240M, V264I, V264T, V264Y, E272Y, K274E, Y278T, 297D, T299A, T299V, T299I, T299H, K326T, L328A, L328H, A330Y, A330L, A330I, I332D, I332E, I332N, and I332Q, wherein numbering is according to the EU index as in Kabat.

The variants may be combined to produce a variant having enhanced properties. Two or more single variants may be combined. In addition, 3, 4, 5, 6 or more variants may be combined, although combinations of about 2 to about 4 variants are preferred. Examples of variant combinations include but are not limited to 1332E, V264I/I332E, S239D/I332E, or S239D/A330L/I332E, wherein numbering is according to the EU index as in Kabat.

Additional variants may be combined with the variants disclosed above. These additional variants include but are not limited to: S298A, K326A, K326S, K326N, K326Q, K326D, K325E, K326W, K326Y, E333A, E333S, K334A, K334E, Y300I, Y300L, Q295K, E294N, S298N, S298V, S298D, D280H, K290S, D280Q, D280Y, K290G, K290T, K290Y, T250Q, T250E, M428L, and M428F, wherein numbering is according to the EU index as in Kabat. These variants may be added as a single variant addition or may be added as more than one addition to the existing variants discussed above.

The FcgRs of the variant proteins of the present invention may be FcgRI, FcgRIIa, FcgRIIb, FcgRIIc, or FcgRIIIa. In one embodiment, it is preferred that the variant protein of the present invention bind with greater affinity to the FcgR relative to a parent protein. In an alternative embodiment, the variant protein of the present invention may bind with reduced affinity to the FcgR relative to a parent protein. More particularly, it is preferred that a variant protein of the present invention binds with greater affinity to human FcgRIIIa relative to a parent protein. It is also preferred, that a variant protein binds with reduced affinity to human FcgRIIb relative to a parent protein.

A variant protein of the present invention may also include an engineered glycoform, an Fc fusion, be chemically modified, aglycosylated, glycosylated, deaminated, and the like, as discussed elsewhere in the specification. Alternatively, insertions may be made in the protein. For example, a glycine may be inserted at position 236 (−236G).

In addition, the variant protein of the present invention may also include amino acid modifications at one or more the following positions: P233E, V234L, A235L, and G327A. More preferably, one or more of these variants may be combined with −236G.

Properties that may be optimized include but are not limited to enhanced or reduced affinity for an FcγR. In a preferred embodiment, the EGFR targeting proteins of the present invention may be optimized to possess enhanced affinity for a human activating FcγR, preferably FcγRI, FcγRIIa, FcγRIIc, FcγRIIIa, and FcγRIIIb, most preferably FcγRIIIa. In an alternately preferred embodiment, the EGFR targeting proteins may be optimized to possess reduced affinity for the human inhibitory receptor FcγRIIb. These preferred embodiments provide EGFR targeting proteins with enhanced therapeutic properties in humans, for example enhanced effector function and greater anti-cancer potency. In an alternate embodiment, the EGFR targeting proteins of the present invention may be optimized to have reduced or ablated affinity for a human FcγR, including but not limited to FcγRI, FcγRIIa, FcγRIIb, FcγRIIc, FcγRIIIa, and FcγRIIIb. These embodiments provide EGFR targeting proteins with enhanced therapeutic properties in humans, for example reduced effector function and reduced toxicity. In other embodiments, EGFR targeting proteins of the present invention provide enhanced affinity for one or more FcγRs, yet reduced affinity for one or more other FcγRs. For example, an EGFR targeting protein of the present invention may have enhanced binding to FcγRIIIa, yet reduced binding to FcγRIIb. Alternately, an EGFR targeting protein of the present invention may have enhanced binding to FcγRIIa and FcγRI, yet reduced binding to FcγRIIb. In yet another embodiment, an EGFR targeting protein of the present invention may have enhanced affinity for FcγRIIb, yet reduced affinity to one or more activating FcγRs.

Preferred embodiments comprise optimization of Fc binding to a human FcγR, however in alternate embodiments the EGFR targeting proteins of the present invention possess enhanced or reduced affinity for FcγRs from nonhuman organisms, including but not limited to rodents and non-human primates. EGFR targeting proteins that are optimized for binding to a nonhuman FcγR may find use in experimentation. For example, mouse models are available for a variety of diseases that enable testing of properties such as efficacy, toxicity, and pharmacokinetics for a given drug candidate. As is known in the art, cancer cells can be grafted or injected into mice to mimic a human cancer, a process referred to as xenografting. Testing of EGFR targeting proteins that comprise EGFR targeting proteins that are optimized for one or more mouse FcγRs, may provide valuable information with regard to the efficacy of the protein, its mechanism of action, and the like. The EGFR targeting proteins of the present invention may also be optimized for enhanced functionality and/or solution properties in aglycosylated form. In a preferred embodiment, the aglycosylated EGFR targeting proteins of the present invention bind an Fc ligand with greater affinity than the aglycosylated form of the parent EGFR targeting protein. Said Fc ligands include but are not limited to FcγRs, C1q, FcRn, and proteins A and G, and may be from any source including but not limited to human, mouse, rat, rabbit, or monkey, preferably human. In an alternately preferred embodiment, the EGFR targeting proteins are optimized to be more stable and/or more soluble than the aglycosylated form of the parent EGFR targeting protein.

EGFR targeting proteins of the invention may comprise modifications that modulate interaction with Fc ligands other than FcγRs, including but not limited to complement proteins, FcRn, and Fc receptor homologs (FcRHs). FcRHs include but are not limited to FcRH1, FcRH2, FcRH3, FcRH4, FcRH5, and FcRH6 (Davis et al., 2002, Immunol. Reviews 190:123-136).

Preferably, the Fc ligand specificity of the EGFR targeting protein of the present invention will determine its therapeutic utility. The utility of a given EGFR targeting protein for therapeutic purposes will depend on the epitope or form of the EGFR target antigen and the disease or indication being treated. For some targets and indications, enhanced FcγR-mediated effector functions may be preferable. This may be particularly favorable for anti-cancer EGFR targeting proteins. Thus EGFR targeting proteins may be used that comprise EGFR targeting proteins that provide enhanced affinity for activating FcγRs and/or reduced affinity for inhibitory FcγRs. For some targets and indications, it may be further beneficial to utilize EGFR targeting proteins that provide differential selectivity for different activating FcγRs; for example, in some cases enhanced binding to FcγRIIa and FcγRIIIa may be desired, but not FcγRI, whereas in other cases, enhanced binding only to FcγRIIa may be preferred. For certain targets and indications, it may be preferable to utilize EGFR targeting proteins that enhance both FcγR-mediated and complement-mediated effector functions, whereas for other cases it may be advantageous to utilize EGFR targeting proteins that enhance either FcγR-mediated or complement-mediated effector functions. For some EGFR targets or cancer indications, it may be advantageous to reduce or ablate one or more effector functions, for example by knocking out binding to C1q, one or more FcγR's, FcRn, or one or more other Fc ligands. For other targets and indications, it may be preferable to utilize EGFR targeting proteins that provide enhanced binding to the inhibitory FcγRIIb, yet WT level, reduced, or ablated binding to activating FcγRs. This may be particularly useful, for example, when the goal of an EGFR targeting protein is to inhibit inflammation or autoimmune disease, or modulate the immune system in some way.

Clearly an important parameter that determines the most beneficial selectivity of a given EGFR targeting protein to treat a given disease is the context of the EGFR targeting protein, that is what type of EGFR targeting protein is being used. Thus the Fc ligand selectivity or specificity of a given EGFR targeting protein will provide different properties depending on whether it composes an antibody, Fc fusion, or an EGFR targeting proteins with a coupled fusion or conjugate partner. For example, toxin, radionucleotide, or other conjugates may be less toxic to normal cells if the EGFR targeting protein that comprises them has reduced or ablated binding to one or more Fc ligands. As another example, in order to inhibit inflammation or autoimmune disease, it may be preferable to utilize an EGFR targeting protein with enhanced affinity for activating FcγRs, such as to bind these FcγRs and prevent their activation. Conversely, an EGFR targeting protein that comprises two or more Fc regions with enhanced FcγRIIb affinity may co-engage this receptor on the surface of immune cells, thereby inhibiting proliferation of these cells. Whereas in some cases an EGFR targeting proteins may engage its target antigen on one cell type yet engage FcγRs on separate cells from the target antigen, in other cases it may be advantageous to engage FcγRs on the surface of the same cells as the target antigen. For example, if an antibody targets an antigen on a cell that also expresses one or more FcγRs, it may be beneficial to utilize an EGFR targeting protein that enhances or reduces binding to the FcγRs on the surface of that cell. This may be the case, for example when the EGFR targeting protein is being used as an anti-cancer agent, and co-engagement of target antigen and FcγR on the surface of the same cell promote signaling events within the cell that result in growth inhibition, apoptosis, or other anti-proliferative effect. Alternatively, antigen and FcγR co-engagement on the same cell may be advantageous when the EGFR targeting protein is being used to modulate the immune system in some way, wherein co-engagement of target antigen and FcγR provides some proliferative or anti-proliferative effect. Likewise, EGFR targeting proteins that comprise two or more Fc regions may benefit from EGFR targeting proteins that modulate FcγR selectivity or specificity to co-engage FcγRs on the surface of the same cell.

The Fc ligand specificity of the EGFR targeting proteins of the present invention can be modulated to create different effector function profiles that may be suited for particular EGFR epitopes, indications or patient populations. Table 1 describes several preferred embodiments of receptor binding profiles that include improvements to, reductions to or no effect to the binding to various receptors, where such changes may be beneficial in certain contexts. The receptor binding profiles in the table could be varied by degree of increase or decrease to the specified receptors. Additionally, the binding changes specified could be in the context of additional binding changes to other receptors such as C1q or FcRn, for example by combining with ablation of binding to C1q to shut off complement activation, or by combining with enhanced binding to C1q to increase complement activation. Other embodiments with other receptor binding profiles are possible, the listed receptor binding profiles are exemplary.

TABLE 1

| | Re- | | |
|---|---|---|---|
| Receptor | ceptor | | |
| binding | binding | | |
| improve- | reduc- | | |
| ment | tion | Cell activity | Therapeutic activity |
| Solely I | — | enhance dendritic cell activity | enhance cell-based |
| | | and uptake, and subsequence | immune response |
| | | presentation of | against target |
| | | antigens; enhance monocyte | |
| | | and macrophage response to | |
| | | antibody | |
| IIIa | | Enhance ADCC and | Increased target cell |
| | | phagocytosis of broad range | lysis |
| | | of cell types | |
| IIIa | IIb | Enhance ADCC and | Increased target cell |
| | | phagocytosis of broad range | lysis |
| | | of cell types | |
| IIb, IIc | | Reduction of activity of all | Enhancement of |
| | | FcR bearing cell types except | target cell lysis |
| | | NK cells and possible | selective for NK cell |
| | | activation of NK cells via IIc | accessible target |
| | | receptor signaling | cells |
| IIb, IIIa | — | Possible NK cell specific | Enhancement of |
| | | activation and enhancement | target cell lysis |
| | | of NK cell mediated ADCC | selective for NK cell |
| | | | accessible target |
| | | | cells |
| IIIb | | Neutrophil mediated | Enhanced target cell |
| | | phagocytosis enhancement | destruction for |
| | | | neutrophil |
| | | | accessible cells |
| FcαR | | Neutrophil mediated | Enhanced target cell |
| | | phagocytosis enhancement | destruction for |
| | | | neutrophil |
| | | | accessible cells |
| I, IIa, IIIa | IIb | enhance dendritic cell activity | enhance cell-based |
| | | and uptake, and subsequence | immune response |
| | | presentation of antigens to T | against target |
| | | cells; enhance monocyte and | |
| | | macrophage response to | |
| | | antibody | |
| IIb | IIIa, IIa, | Reduction in activity of | Eliminate or reduce |
| | I | monocytes, macrophages, | cell-mediated |
| | | neutrophils, NK, dendritic and | cytotoxicity against |
| | | other gamma receptor | target bearing cells |
| | | bearing cells | |

The presence of different polymorphic forms of FcγRs provides yet another parameter that impacts the therapeutic utility of the EGFR targeting proteins of the present invention. Whereas the specificity and selectivity of a given EGFR targeting protein for the different classes of FcγRs significantly affects the capacity of an EGFR targeting protein to target a given antigen for treatment of a given disease, the specificity or selectivity of an EGFR targeting protein for different polymorphic forms of these receptors may in part determine which research or pre-clinical experiments may be appropriate for testing, and ultimately which patient populations may or may not respond to treatment. Thus the specificity or selectivity of EGFR targeting proteins of the present invention to Fc ligand polymorphisms, including but not limited to FcγR, C1q, FcRn, and FcRH polymorphisms, may be used to guide the selection of valid research and pre-clinical experiments, clinical trial design, patient selection, dosing dependence, and/or other aspects concerning clinical trials.

The EGFR targeting proteins of the present invention may be combined with other amino acid modifications in the Fc region that provide altered or optimized interaction with one or more Fc ligands, including but not limited to FcγRs, C1q, FcRn, FcR homologues, and/or as yet undiscovered Fc ligands. Additional modifications may provide altered or optimized affinity and/or specificity to the Fc ligands. Additional modifications may provide altered or optimized effector functions, including but not limited to ADCC, ADCP, CDC, and/or serum half-life. Such combination may provide additive, synergistic, or novel properties in antibodies or Fc fusions. In one embodiment, the EGFR targeting proteins of the present invention may be combined with known Fc variants (Duncan et al., 1988, Nature 332:563-564; Lund et al., 1991, J Immunol 147:2657-2662; Lund et al., 1992, Mol Immunol 29:53-59; Alegre et al., 1994, Transplantation 57:1537-1543; Hutchins et al., 1995, Proc Natl Acad Sci USA 92:11980-11984; Jefferis et al., 1995, Immunol Lett 44:1 11-117; Lund et al., 1995, Faseb J 9:115-119; Jefferis et al., 1996, Immunol Lett 54:101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164:1925-1933; Xu et al., 2000, Cell Immunol 200:16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490; Hinton et al., 2004, J Biol Chem 279:6213-6216) (U.S. Pat. No. 5,624,821; U.S. Pat. No. 5,885,573; U.S. Pat. No. 6,194,551; PCT WO 00/42072; PCT WO 99/58572; US 2004/0002587 A1), U.S. Pat. No. 6,737,056, PCT US2004/000643, U.S. Ser. No. 10/370,749, and PCT/US2004/005112). For example, as described in U.S. Pat. No. 6,737,056, PCT US2004/000643, U.S. Ser. No. 10/370,749, and PCT/US2004/005112, the substitutions S298A, S298D, K326E, K326D, E333A, K334A, and P396L provide optimized FcγR binding and/or enhanced ADCC. Furthermore, as disclosed in Idusogie et al., 2001, J. Immunology 166:2571-2572, substitutions K326W, K326Y, and E333S provide enhanced binding to the complement protein C1q and enhanced CDC. Finally, as described in Hinton et al., 2004, J. Biol. Chem. 279(8): 6213-6216, substitutions T250Q, T250E, M428L, and M428F provide enhanced binding to FcRn and improved pharmacokinetics.

Because the binding sites for FcγRs, C1q, and FcRn reside in the Fc region, the differences between the IgGs in the Fc region are likely to contribute to differences in FcγR- and C1q-mediated effector functions. It is also possible that the modifications can be made in other non-Fc regions of an EGFR targeting protein, including for example the Fab and hinge regions of an antibody, or the Fc fusion partner of an Fc fusion. For example, as disclosed in U.S. Ser. Nos. 60/573,302; 60/585,328; 60/586,837; 60/589,906; 60/599,741; 60/607,398; 60/614,944; and 60/619,409, the Fab and hinge regions of an antibody may impact effector functions such as antibody dependent cell-mediated cytotoxicity (ADCC), antibody dependent cell-mediated phagocytosis (ADCP), and complement dependent cytotoxicity (CDC). Thus modifications outside the Fc region of an EGFR targeting protein of the present invention are contemplated. For example, anti-EGFR antibodies of the present invention may comprise one or more amino acid modifications in the VL, CL, VH, CH1, and/or hinge regions of an antibody.

Other modifications may provide additional or novel binding determinants into an EGFR targeting protein, for example additional or novel Fc receptor binding sites, for example as described in U.S. Ser. No. 60/531,752, filed Dec. 22, 2003, entitled “EGFR targeting proteins with novel Fc receptor binding sites”. In one embodiment, an EGFR targeting protein of one antibody isotype may be engineered such that it binds to an Fc receptor of a different isotype. This may be particularly applicable when the Fc binding sites for the respective Fc receptors do not significantly overlap. For example, the structural determinants of IgA binding to FcγR1 may be engineered into an IgG EGFR targeting protein.

The EGFR targeting proteins of the present invention may comprise modifications that modulate the in vivo pharmacokinetic properties of an EGFR targeting protein. These include, but are not limited to, modifications that enhance affinity for the neonatal Fc receptor FcRn (See for example, U.S. Ser. No. 10/020,354; WO 2001 US0048432; EP 2001000997063; U.S. Pat. No. 6,277,375; U.S. Ser. No. 09/933,497; WO 1997US0003321; U.S. Pat. No. 6,737,056; WO 2000US0000973; Shields et al. J. Biol. Chem., 276(9), 6591-6604 (2001); Zhou et al. J. Mol. Biol., 332, 901-913 (2003)). These further include modifications that modify FcRn affinity in a pH-specific manner. In some embodiments, where enhanced in vivo half-life is desired, modifications that specifically enhance FcRn affinity at lower pH (5.5-6) relative to higher pH (7-8) are preferred (Hinton et al. J. Biol. Chem. 279(8), 6213-6216 (2004); Dall' Acqua et al. J. Immuno. 169, 5171-5180 (2002); Ghetie et al. Nat. Biotechnol., 15(7), 637-640 (1997); WO 2003US0033037; WO 2004US0011213). For example, as described in Hinton et al., 2004, “Engineered Human IgG Antibodies with Longer Serum Half-lives in Primates” J. Biol. Chem. 279(8): 6213-6216, substitutions T250Q, T250E, M428L, and M428F provide enhanced binding to FcRn and improved pharmacokinetics. Additionally preferred modifications are those that maintain the wild-type Fc's improved binding at lower pH relative to the higher pH. In alternative embodiments, where rapid in vivo clearance is desired, modifications that reduce affinity for FcRn are preferred. (See for example, U.S. Pat. No. 6,165,745; WO 1993US0003895; EP 1993000910800; WO 1997US0021437; Medesan et al., J. Immunol., 158(5), 2211-2217 (1997); Ghetie and Ward, Annu. Rev. Immunol., 18, 739-766 (2000); Martin et al. Molecular Cell, 7, 867-877 (2001); Kim et al. Eur. J. Immunol. 29, 2819-2825 (1999)).

EGFR targeting proteins of the present invention may comprise one or more modifications that provide optimized properties that are not specifically related to effector function per se. Said modifications may be amino acid modifications, or may be modifications that are made enzymatically or chemically. Such modification(s) likely provide some improvement in the EGFR targeting protein, for example an enhancement in its stability, solubility, function, or clinical use. The present invention contemplates a variety of improvements that made be made by coupling the EGFR targeting proteins of the present invention with additional modifications.

In a preferred embodiment, the EGFR targeting proteins of the present invention may comprise modifications to reduce immunogenicity in humans. In a most preferred embodiment, the immunogenicity of an EGFR targeting protein of the present invention is reduced using a method described in U.S. Ser. Nos. 60/581,613; 60/601,665; 60/619,483; and U.S. Ser. No. 10/______, entitled “Methods of Generating Variant Proteins with Increased Host String Content and Compositions Thereof”, filed on Dec. 6, 2004. In alternate embodiments, the antibodies of the present invention are humanized (Clark, 2000, Immunol Today 21 :397-402). By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (See, for example, U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; and U.S. Pat. No. 6,407,213). The humanized antibody optimally also will comprise at least a portion of an immunoglobulin constant region, typically that of a human immunoglobulin, and thus will typically comprise a human Fc region. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et a., 1992, Proc Natl Acad Sci USA 89:42859, Presta et al., 1997, Cancer Res. 57(20): 4593-9; Gorman et al., 1991, Proc. Natl. Acad. Sci. USA 88:4181-4185; O'Connor et al., 1998, Protein Eng 11:321-8. Humanization or other methods of reducing the immunogenicity of nonhuman antibody variable regions may include resurfacing methods, as described for example in Roguska et al., 1994, Proc. Natl. Acad. Sci. USA 91:969-973. In one embodiment, selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16): 10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10): 753-759. Other humanization methods may involve the grafting of only parts of the CDRs, including but not limited to methods described in U.S. Ser. No. 09/810,502; Tan et al., 2002, J. Immunol. 169:1119-1125; De Pascalis et al., 2002, J. Immunol. 169:3076-3084. Structure-based methods may be employed for humanization and affinity maturation, for example as described in U.S. Ser. No. 10/153,159 and related applications.

Modifications to reduce immunogenicity may include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an EGFR targeting protein of the present invention. See for example WO 98/52976; WO 02/079232; WO 00/3317; U.S. Ser. No. 09/903,378; U.S. Ser. No. 10/039,170; U.S. Ser. No. 60/222,697; U.S. Ser. No. 10/339788; PCT WO 01/21823; and PCT WO 02/00165; Mallios, 1999, Bioinformatics 15: 432439; Mallios, 2001, Bioinformatics 17: 942-948; Sturniolo et al., 1999, Nature Biotech. 17: 555-561; WO 98/59244; WO 02/069232; WO 02/77187; Marshall et al., 1995, J. Immunol. 154: 5927-5933; and Hammer et al., 1994, J. Exp. Med. 180: 2353-2358. Sequence-based information can be used to determine a binding score for a given peptide—MHC interaction (see for example Mallios, 1999, Bioinformatics 15: 432-439; Mallios, 2001, Bioinformatics 17: p942-948; Sturniolo et. al., 1999, Nature Biotech. 17: 555-561). It is possible to use structure-based methods in which a given peptide is computationally placed in the peptide-binding groove of a given MHC molecule and the interaction energy is determined (for example, see WO 98/59244 and WO 02/069232). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (See for example WO 02/77187). In a preferred embodiment, MHC-binding propensity scores are calculated for each 9-residue frame along the protein sequence using a matrix method (see Sturniolo et. al., supra; Marshall et. al., 1995, J. Immunol. 154: 5927-5933, and Hammer et. al., 1994, J. Exp. Med. 180: 2353-2358). It is also possible to consider scores for only a subset of these residues, or to consider also the identities of the peptide residues before and after the 9-residue frame of interest. The matrix comprises binding scores for specific amino acids interacting with the peptide binding pockets in different human class 11 MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”. In an alternate embodiment, additional amino acid modifications may be engineered to reduce the propensity of the intact molecule to interact with B cell receptors and circulating antibodies.

Anti-EGFR antibodies and Fc fusions of the present invention may comprise amino acid modifications in one or more regions outside the Fc region, for example the antibody Fab region or the Fc fusion partner, that provide optimal properties. In one embodiment, the variable region of an antibody of the present invention may be affinity matured, that is to say that amino acid modifications have been made in the VH and/or VL domains of the antibody to enhance binding of the antibody to its target antigen. Likewise, modifications may be made in the Fc fusion partner to enhance affinity of the Fc fusion for its target antigen. Such types of modifications may improve the association and/or the dissociation kinetics for binding to the target antigen. Other modifications include those that improve selectivity for target antigen vs. alternative targets. These include modifications that improve selectivity for antigen expressed on target vs. non-target cells. Other improvements to the target recognition properties may be provided by additional modifications. Such properties may include, but are not limited to, specific kinetic properties (i.e. association and dissociation kinetics), selectivity for the particular target versus alternative targets, and selectivity for a specific form of target versus alternative forms. Examples include full-length versus splice variants, cell-surface vs. soluble forms, selectivity for various polymorphic variants, or selectivity for specific conformational forms of the EGFR target.

EGFR targeting proteins of the invention may comprise one or more modifications that provide reduced or enhanced internalization of an EGFR targeting protein. In one embodiment, EGFR targeting proteins of the present invention can be utilized or combined with additional modifications in order to reduce the cellular internalization of an EGFR targeting protein that occurs via interaction with one or more Fc ligands. This property might be expected to enhance effector function, and potentially reduce immunogenicity of the EGFR targeting proteins of the invention. Alternatively, EGFR targeting proteins of the present EGFR targeting proteins of the present invention can be utilized directly or combined with additional modifications in order to enhance the cellular internalization of an EGFR targeting protein that occurs via interaction with one or more Fc ligands. For example, in a preferred embodiment, an EGFR targeting protein is used that provides enhanced binding to FcγRI, which is expressed on dendritic cells and active early in immune response. This strategy could be further enhanced by combination with additional modifications, either within the EGFR targeting protein or in an attached fusion or conjugate partner, that promote recognition and presentation of Fc peptide fragments by MHC molecules. These strategies are expected to enhance target antigen processing and thereby improve antigenicity of the target antigen (Bonnerot and Amigorena, 1999, Immunol Rev. 172:279-84), promoting an adaptive immune response and greater target cell killing by the human immune system. These strategies may be particularly advantageous when the targeted antigen is shed from the cellular surface. An additional application of these concepts arises with idiotype vaccine immunotherapies, in which clone-specific antibodies produced by a patient's lymphoma cells are used to vaccinate the patient.

In a preferred embodiment, modifications are made to improve biophysical properties of the EGFR targeting proteins of the present invention, including but not limited to stability, solubility, and oligomeric state. Modifications can include, for example, substitutions that provide more favorable intramolecular interactions in the EGFR targeting protein such as to provide greater stability, or substitution of exposed nonpolar amino acids with polar amino acids for higher solubility. A number of optimization goals and methods are described in U.S. Ser. No. 10/379,392 that may find use for engineering additional modifications to further optimize the EGFR targeting proteins of the present invention. The EGFR targeting proteins of the present invention can also be combined with additional modifications that reduce oligomeric state or size, such that tumor penetration is enhanced, or in vivo clearance rates are increased as desired.

Other modifications to the EGFR targeting proteins of the present invention include those that enable the specific formation or homodimeric or homomultimeric molecules. Such modifications include but are not limited to engineered disulfides, as well as chemical modifications or aggregation methods which may provide a mechanism for generating covalent homodimeric or homomultimers. For example, methods of engineering and compositions of such molecules are described in Kan et al., 2001, J Immunol., 2001, 166: 1320-1326; Stevenson et al., 2002, Recent Results Cancer Res. 159: 104-12; U.S. Pat. No. 5,681,566; Caron et al., 1992, J. Exp. Med. 176:1191-1195, and Shopes, 1992, J. Immunol. 148(9): 2918-22. Additional modifications to the variants of the present invention include those that enable the specific formation or heterodimeric, heteromultimeric, bifunctional, and/or multifunctional molecules. Such modifications include, but are not limited to, one or more amino acid substitutions in the CH3 domain, in which the substitutions reduce homodimer formation and increase heterodimer formation. For example, methods of engineering and compositions of such molecules are described in Atwell et al., 1997, J. Mol. Biol. 270(1):26-35, and Carter et al., 2001, J. Immunol. Methods 248:7-15. Additional modifications include modifications in the hinge and CH3 domains, in which the modifications reduce the propensity to form dimers.

In further embodiments, the EGFR targeting proteins of the present invention comprise modifications that remove proteolytic degradation sites. These may include, for example, protease sites that reduce production yields, as well as protease sites that degrade the administered protein in vivo. In a preferred embodiment, additional modifications are made to remove covalent degradation sites such as deamidation (i.e. deamidation of glutaminyl and asparaginyl residues to the corresponding glutamyl and aspartyl residues), oxidation, and proteolytic degradation sites. Deamidation sites that are particular useful to remove are those that have enhance propensity for deamidation, including, but not limited to asparaginyl and gltuamyl residues followed by glycines (NG and QG motifs, respectively). In such cases, substitution of either residue can significantly reduce the tendency for deamidation. Common oxidation sites include methionine and cysteine residues. Other covalent modifications, that can either be introduced or removed, include hydroxylation of proline and lysine, phosphorylation of hydroxyl groups of seryl or threonyl residues, methylation of the amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group. Additional modifications also may include but are not limited to posttranslational modifications such as N-linked or O-linked glycosylation and phosphorylation.

Modifications may include those that improve expression and/or purification yields from hosts or host cells commonly used for production of biologics. These include, but are not limited to various mammalian cell lines (e.g. CHO), yeast cell lines, bacterial cell lines, and plants. Additional modifications include modifications that remove or reduce the ability of heavy chains to form inter-chain disulfide linkages. Additional modifications include modifications that remove or reduce the ability of heavy chains to form intra-chain disulfide linkages.

The EGFR targeting proteins of the present invention may comprise modifications that include the use of unnatural amino acids incorporated using, for example, the technologies developed by Schultz and colleagues, including but not limited to methods described by Cropp & Shultz, 2004, Trends Genet. 20(12): 625-30, Anderson et al., 2004, Proc. Natl. Acad. Sci. U.S.A. 101(2): 7566-71, Zhang et al., 2003, 303(5656): 371-3, and Chin et al., 2003, Science 301(5635): 964-7. In some embodiments, these modifications enable manipulation of various functional, biophysical, immunological, or manufacturing properties discussed above. In additional embodiments, these modifications enable additional chemical modification for other purposes. Other modifications are contemplated herein. For example, the EGFR targeting protein may be linked to one of a variety of nonproteinaceous polymers, e.g., polyethylene glycol (PEG), polypropylene glycol, polyoxyalkylenes, or copolymers of polyethylene glycol and polypropylene glycol. Additional amino acid modifications may be made to enable specific or non-specific chemical or posttranslational modification of the EGFR targeting proteins. Such modifications, include, but are not limited to PEGylation and glycosylation. Specific substitutions that can be utilized to enable PEGylation include, but are not limited to, introduction of novel cysteine residues or unnatural amino acids such that efficient and specific coupling chemistries can be used to attach a PEG or otherwise polymeric moiety. Introduction of specific glycosylation sites can be achieved by introducing novel N-X-T/S sequences into the EGFR targeting proteins of the present invention.

In one embodiment, the EGFR targeting proteins of the present invention comprise one or more engineered glycoforms. By “engineered glycoform” as used herein is meant a carbohydrate composition that is covalently attached to an EGFR targeting protein, wherein said carbohydrate composition differs chemically from that of a parent EGFR targeting protein. Engineered glycoforms may be useful for a variety of purposes, including but not limited to enhancing or reducing effector function. Engineered glycoforms may be generated by a variety of methods known in the art (Umaña et al., 1999, Nat Biotechnol 17:176-180; Davies et al., 2001, Biotechnol Bioeng 74:288-294; Shields et al., 2002, J Biol Chem 277:26733-26740; Shinkawa et al., 2003, J Biol Chem 278:3466-3473); (U.S. Pat. No. 6,602,684; U.S. Ser. No. 10/277,370; U.S. Ser. No. 10/113,929; PCT WO 00/61739A1; PCT WO 01/29246A1; PCT WO 02/31140A1; PCT WO 02/30954A1); (Potelligent™ technology [Biowa, Inc., Princeton, N.J.]; GlycoMAb™ glycosylation engineering technology [GLYCART biotechnology A G, Zürich, Switzerland]). Many of these techniques are based on controlling the level of fucosylated and/or bisecting oligosaccharides that are covalently attached to the Fc region, for example by expressing an EGFR targeting protein in various organisms or cell lines, engineered or otherwise (for example Lec-13 CHO cells or rat hybridoma YB2/0 cells), by regulating enzymes involved in the glycosylation pathway (for example FUT8 [α1,6-fucosyltranserase] and/or β1-4-N-acetylglucosaminyltransferase III [GnTIII]), or by modifying carbohydrate(s) after the EGFR targeting protein has been expressed. Engineered glycoform typically refers to the different carbohydrate or oligosaccharide; thus an EGFR targeting protein, for example an anti-EGFR antibody or Fc fusion, may comprise an engineered glycoform. Alternatively, engineered glycoform may refer to the EGFR targeting protein that comprises the different carbohydrate or oligosaccharide.

The EGFR targeting proteins of the present invention may be fused or conjugated to one or more other molecules or polypeptides. Conjugate and fusion partners may be any molecule, including small molecule chemical compounds and polypeptides. For example, a variety of antibody conjugates and methods are described in Trail et al., 1999, Curr. Opin. Immunol. 11:584-588. Possible conjugate partners include but are not limited to cytokines, cytotoxic agents, toxins, radioisotopes, chemotherapeutic agent, anti-angiogenic agents, tyrosine kinase inhibitors, and other therapeutically active agents. In some embodiments, conjugate partners may be thought of more as payloads, that is to say that the goal of a conjugate is targeted delivery of the conjugate partner to a targeted cell, for example a cancer cell or immune cell, by the EGFR targeting protein. Thus, for example, the conjugation of a toxin to an anti-EGFR antibody or Fc fusion targets the delivery of said toxin to cells expressing the EGFR antigen. As will be appreciated by one skilled in the art, in reality the concepts and definitions of fusion and conjugate are overlapping. The designation of an EGFR targeting protein as a fusion or conjugate is not meant to constrain it to any particular embodiment of the present invention. Rather, these terms are used loosely to convey the broad concept that any EGFR targeting protein of the present invention may be linked genetically, chemically, or otherwise, to one or more polypeptides or molecules to provide some desirable property.

In one embodiment, the EGFR targeting proteins of the present invention are fused or conjugated to a cytokine. By “cytokine” as used herein is meant a generic term for proteins released by one cell population that act on another cell as intercellular mediators. For example, as described in Penichet et al., 2001, J. Immunol. Methods 248:91-101, cytokines may be fused to antibody to provide an array of desireable properties. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; C5a; and other polypeptide factors including LIF and kit ligand (KL). As used herein, the term cytokine includes proteins from natural sources or from recombinant cell culture, and biologically active equivalents of the native sequence cytokines.

In an alternate embodiment, the EGFR targeting proteins of the present invention are fused, conjugated, or operably linked to a toxin, including but not limited to small molecule toxins and enzymatically active toxins of bacterial, fungal, plant or animal origin, including fragments and/or variants thereof. For example, a variety of immunotoxins and immunotoxin methods are described in Thrush et al., 1996, Ann. Rev. Immunol. 14:49-71. Small molecule toxins include but are not limited to calicheamicin, maytansine (U.S. Pat. No. 5,208,020), trichothene, and CC1065. In one embodiment of the invention, the anti-EGFR antibody or Fc fusion is conjugated to one or more maytansine molecules (e.g. about 1 to about 10 maytansine molecules per antibody molecule). Maytansine may, for example, be converted to May-SS-Me, which may be reduced to May-SH3 and reacted with modified antibody or Fc fusion (Chari et al., 1992, Cancer Research 52: 127-131) to generate a maytansinoid-antibody or maytansinoid-Fc fusion conjugate. Another conjugate of interest comprises an anti-EGFR antibody or Fc fusion conjugated to one or more calicheamicin molecules. The calicheamicin family of antibiotics are capable of producing double-stranded DNA breaks at sub-picomolar concentrations. Structural analogues of calicheamicin that may be used include but are not limited to γ_{1 hu 1}, α₂¹, α₃, N-acetyl-γ₁¹, PSAG, and ⊖¹₁, (Hinman et al., 1993, Cancer Research 53:3336-3342; Lode et al., 1998, Cancer Research 58:2925-2928) (U.S. Pat. No. 5,714,586; U.S. Pat. No. 5,712,374; U.S. Pat. No. 5,264,586; U.S. Pat. No. 5,773,001). Dolastatin 10 analogs such as auristatin E (AE) and monomethylauristatin E (MMAE) may find use as conjugates for the EGFR targeting proteins of the present invention (Doronina et al., 2003, Nat Biotechnol 21(7): 778-84; Francisco et al., 2003 Blood 102(4): 1458-65). Useful enyzmatically active toxins include but are not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, PCT WO 93/21232. The present invention further contemplates a conjugate between an EGFR targeting protein of the present invention and a compound with nucleolytic activity, for example a ribonuclease or DNA endonuclease such as a deoxyribonuclease (Dnase).

In an alternate embodiment, an EGFR targeting protein of the present invention may be fused, conjugated, or operably linked to a radioisotope to form a radioconjugate. A variety of radioactive isotopes are available for the production of radioconjugate antibodies and Fc fusions. Examples include, but are not limited to, At211, I131, I125, Y90, Re186, Re188, Sm153, Bi212, P32, and radioactive isotopes of Lu. See for example, reference.

In yet another embodiment, an EGFR targeting protein of the present invention may be conjugated to a “receptor” (such streptavidin) for utilization in tumor pretargeting wherein the EGFR targeting protein-receptor conjugate is administered to the patient, followed by removal of unbound conjugate from the circulation using a clearing agent and then administration of a “ligand” (e.g. avidin) which is conjugated to a cytotoxic agent (e.g. a radionucleotide). In an alternate embodiment, the EGFR targeting protein is conjugated or operably linked to an enzyme in order to employ Antibody Dependent Enzyme Mediated Prodrug Therapy (ADEPT). ADEPT may be used by conjugating or operably linking the EGFR targeting protein to a prodrug-activating enzyme that converts a prodrug (e.g. a peptidyl chemotherapeutic agent, see PCT WO 81/01145) to an active anti-cancer drug. See, for example, PCT WO 88/07378 and U.S. Pat. No. 4,975,278. The enzyme component of the immunoconjugate useful for ADEPT includes any enzyme capable of acting on a prodrug in such a way so as to covert it into its more active, cytotoxic form. Enzymes that are useful in the method of this invention include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as .beta.-galactosidase and neuramimidase useful for converting glycosylated prodrugs into free drugs; beta-lactamase useful for converting drugs derivatized with .alpha.-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, for example, Massey, 1987, Nature 328: 457-458). EGFR targeting protein-abzyme conjugates can be prepared for delivery of the abzyme to a tumor cell population. A variety of additional conjugates are contemplated for the EGFR targeting proteins of the present invention. A variety of chemotherapeutic agents, anti-angiogenic agents, tyrosine kinase inhibitors, and other therapeutic agents are described below, which may find use as EGFR targeting protein conjugates.

Alternatively, fusion and conjugate partners also include Fc polypeptides. Thus, an EGFR targeting protein may be a multimeric Fc polypeptide, comprising two or more Fc regions. The advantage of such a molecule is that it provides multiple binding sites for Fc receptors with a single protein molecule. In one embodiment, Fc regions may be linked using a chemical engineering approach. For example, Fab's and Fc's may be linked by thioether bonds originating at cysteine residues in the hinges, generating molecules such as FabFc₂ (Kan et al., 2001, J. Immunol., 2001,166: 1320-1326; Stevenson et al., 2002, Recent Results Cancer Res. 159: 104-12; U.S. Pat. No. 5,681,566). Fc regions may be linked using disulfide engineering and/or chemical cross-linking, for example as described in Caron et al., 1992, J. Exp. Med. 176:1191-1195, and Shopes, 1992, J. Immunol. 148(9): 2918-22. In a preferred embodiment, Fc regions may be linked genetically. For example multiple Cγ2 domains have been fused between the Fab and Fc regions of an antibody (White et al., 2001, Protein Expression and Purification 21: 446-455). In a preferred embodiment, Fc regions in an EGFR targeting protein are linked genetically to generated tandemly linked Fc regions as described in U.S. Ser. No. 60/531,752, filed Dec. 22, 2003, entitled “Fc polypeptides with novel Fc receptor binding sites”. Tandemly linked Fc polypeptides may comprise two or more Fc regions, preferably one to three, and most preferably two Fc regions. It may be advantageous to explore a number of engineering constructs in order to obtain homo- or hetero-tandemly linked EGFR targeting proteins with the most favorable structural and functional properties. Tandemly linked EGFR targeting proteins may be homo-tandemly linked EGFR targeting proteins, i.e., an EGFR targeting protein of one isotype is fused genetically to another EGFR targeting protein of the same isotype. It is anticipated that because there are multiple FcγR, C1q, and/or FcRn binding sites on tandemly linked Fc polypeptides, effector functions and/or pharmacokinetics may be enhanced. In an alternate embodiment, EGFR targeting proteins from different isotypes may be tandemly linked, referred to as hetero-tandemly linked EGFR targeting proteins. For example, because of the capacity to target FcγR and FcαRI receptors, an EGFR targeting protein that binds both FcγRs and FcαRI may provide a significant clinical improvement.

Fusion and conjugate partners may be linked to any region of an EGFR targeting protein of the present invention, including at the N- or C-termini, or at some residue in-between the termini. In a preferred embodiment, a fusion or conjugate partner is linked at the N- or C-terminus of the EGFR targeting protein, most preferably the N-terminus. A variety of linkers may find use in the present invention to covalently link EGFR targeting proteins to a fusion or conjugate partner or generate an Fc fusion. By “linker”, “linker sequence”, “spacer”, “tethering sequence” or grammatical equivalents thereof, herein is meant a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 50 amino acid residues. Preferably, the linker is from about 1 to 30 amino acids in length, with linkers of 1 to 20 amino acids in length being most preferred. In addition, the amino acid residues selected for inclusion in the linker peptide should exhibit properties that do not interfere significantly with the activity of the polypeptide. Thus, the linker peptide on the whole should not exhibit a charge that would be inconsistent with the activity of the polypeptide, or interfere with internal folding, or form bonds or other interactions with amino acid residues in one or more of the monomers that would seriously impede the binding of receptor monomer domains. Useful linkers include glycine-serine polymers (including, for example, (GS)n, (GSGGS)n (GGGGS)n and (GGGS)n, where n is an integer of at least one), glycine-alanine polymers, alanine-serine polymers, and other flexible linkers such as the tether for the shaker potassium channel, and a large variety of other flexible linkers, as will be appreciated by those in the art. Glycine-serine polymers are preferred since both of these amino acids are relatively unstructured, and therefore may be able to serve as a neutral tether between components. Secondly, serine is hydrophilic and therefore able to solubilize what could be a globular glycine chain. Third, similar chains have been shown to be effective in joining subunits of recombinant proteins such as single chain antibodies. Suitable linkers may also be identified by screening databases of known three-dimensional structures for naturally occurring motifs that can bridge the gap between two polypeptide chains. In a preferred embodiment, the linker is not immunogenic when administered in a human patient. Thus linkers may be chosen such that they have low immunogenicity or are thought to have low immunogenicity. For example, a linker may be chosen that exists naturally in a human. In a most preferred embodiment, the linker has the sequence of the hinge region of an antibody, that is the sequence that links the antibody Fab and Fc regions; alternatively the linker has a sequence that comprises part of the hinge region, or a sequence that is substantially similar to the hinge region of an antibody. Another way of obtaining a suitable linker is by optimizing a simple linker, e.g., (Gly4Ser)n, through random mutagenesis. Alternatively, once a suitable polypeptide linker is defined, additional linker polypeptides can be created to select amino acids that more optimally interact with the domains being linked. Other types of linkers that may be used in the present invention include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyidithiol) propionate (SPDP), succinimidyl-4-(N-maleimidomethyl)cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., 1971, Science 238:1098. Chemical linkers may enable chelation of an isotope. For example, Carbon¹⁴-labeled 1-isothiocyanatobenzyl-3-methyldiethylene triaminepentaacetic acid (MX-DTPA) is an exemplary chelating agent for conjugation of radionucleotide to the antibody (see, for example, PCT WO 94/11026). The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the EGFR targeting proteins of the present invention to a fusion or conjugate partner to generate an anti-EGFR Fc fusion, or to link the EGFR targeting proteins of the present invention to a conjugate.

Experimental Production of EGFR Targeting Proteins

The present invention provides methods for producing and experimentally testing EGFR targeting proteins. The described methods are not meant to constrain the present invention to any particular application or theory of operation. Rather, the provided methods are meant to illustrate generally that one or more EGFR targeting proteins may be produced and experimentally tested to obtain variant EGFR targeting proteins. General methods for antibody molecular biology, expression, purification, and screening are described in Antibody Engineering, edited by Duebel & Kontermann, Springer-Verlag, Heidelberg, 2001; and Hayhurst & Georgiou, 2001, Curr Opin Chem Biol 5:683-689; Maynard & Georgiou, 2000, Annu Rev Biomed Eng 2:339-76; Antibodies: A Laboratory Manual by Harlow & Lane, New York: Cold Spring Harbor Laboratory Press, 1988.

In one embodiment of the present invention, nucleic acids are created that encode the EGFR targeting proteins, and that may then be cloned into host cells, expressed and assayed, if desired. Thus, nucleic acids, and particularly DNA, may be made that encode each protein sequence. These practices are carried out using well-known procedures. For example, a variety of methods that may find use in the present invention are described in Molecular Cloning—A Laboratory Manual, 3^{rd} Ed. (Maniatis, Cold Spring Harbor Laboratory Press, New York, 2001), and Current Protocols in Molecular Biology (John Wiley & Sons). As will be appreciated by those skilled in the art, the generation of exact sequences for a library comprising a large number of sequences is potentially expensive and time consuming. Accordingly, there are a variety of techniques that may be used to efficiently generate libraries of the present invention. Such methods that may find use in the present invention are described or referenced in U.S. Pat. No. 6,403,312; U.S. Ser. No. 09/782,004; U.S. Ser. No. 09/927,790; U.S. Ser. No. 10/218,102; PCT WO 01/40091; and PCT WO 02/25588. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode EGFR targeting proteins.

The EGFR targeting proteins of the present invention may be produced by culturing a host cell transformed with nucleic acid, preferably an expression vector, containing nucleic acid encoding the EGFR targeting proteins, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC®) cell line catalog, available from the American Type Culture Collection.

In a preferred embodiment, the EGFR targeting proteins are expressed in mammalian expression systems, including systems in which the expression constructs are introduced into the mammalian cells using virus such as retrovirus or adenovirus. Any mammalian cells may be used, with human, mouse, rat, hamster, and primate cells being particularly preferred. Suitable cells also include known research cells, including but not limited to Jurkat T cells, NIH3T3, CHO, BHK, COS, HEK293, PER C.6, HeLa, Sp2/0, NS0 cells and variants thereof. In an alternately preferred embodiment, library proteins are expressed in bacterial cells. Bacterial expression systems are well known in the art, and include Escherichia coli (E. coli), Bacillus subtilis, Streptococcus cremoris, and Streptococcus lividans. In alternate embodiments, EGFR targeting proteins are produced in insect cells (e.g. Sf21/Sf9, Trichoplusia ni Bti-Tn5b1-4) or yeast cells (e.g. S. cerevisiae, Pichia, etc). In an alternate embodiment, EGFR targeting proteins are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the EGFR targeting proteins may be produced by chemical synthesis methods. Also transgenic expression systems both animal (e.g. cow, sheep or goat milk, embryonated hen's eggs, whole insect larvae, etc.) and plant (e.g. corn, tobacco, duckweed, etc.)

The nucleic acids that encode the EGFR targeting proteins of the present invention may be incorporated into an expression vector in order to express the protein. A variety of expression vectors may be utilized for protein expression. Expression vectors may comprise self-replicating extra-chromosomal vectors or vectors which integrate into a host genome. Expression vectors are constructed to be compatible with the host cell type. Thus expression vectors which find use in the present invention, include but are not limited to, those which enable protein expression in mammalian cells, bacteria, insect cells, yeast, and in in vitro systems. As is known in the art, a variety of expression vectors are available, commercially or otherwise, that may find use in the present invention for expressing EGFR targeting proteins.

Expression vectors typically comprise a protein operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the EGFR targeting protein, and are typically appropriate to the host cell used to express the protein. In general, the transcriptional and translational regulatory sequences may include promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

EGFR targeting proteins may be operably linked to a fusion partner to enable targeting of the expressed protein, purification, screening, display, and the like. Fusion partners may be linked to the EGFR targeting protein sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS. A fusion partner may be a targeting or signal sequence that directs EGFR targeting protein and any associated fusion partners to a desired cellular location or to the extracellular media. As is known in the art, certain signaling sequences may target a protein to be either secreted into the growth media, or into the periplasmic space, located between the inner and outer membrane of the cell. A fusion partner may also be a sequence that encodes a peptide or protein that enables purification and/or screening. Such fusion partners include but are not limited to polyhistidine tags (His-tags) (for example H₆ and H₁₀ or other tags for use with Immobilized Metal Affinity Chromatography (IMAC) systems (e.g. Ni⁺² affinity columns)), GST fusions, MBP fusions, Strep-tag, the BSP biotinylation target sequence of the bacterial enzyme BirA, and epitope tags which are targeted by antibodies (for example c-myc tags, flag-tags, and the like). As will be appreciated by those skilled in the art, such tags may be useful for purification, for screening, or both. For example, an EGFR targeting protein may be purified using a His-tag by immobilizing it to a Ni⁺² affinity column, and then after purification the same His-tag may be used to immobilize the antibody to a Ni⁺² coated plate to perform an ELISA or other binding assay (as described below). A fusion partner may enable the use of a selection method to screen EGFR targeting proteins (see below). Fusion partners that enable a variety of selection methods are well-known in the art, and all of these find use in the present invention. For example, by fusing the members of an EGFR targeting protein library to the gene III protein, phage display can be employed (Kay et al., Phage display of peptides and proteins: a laboratory manual, Academic Press, San Diego, Calif., 1996; Lowman et al., 1991, Biochemistry 30:10832-10838; Smith, 1985, Science 228:1315-1317). Fusion partners may enable EGFR targeting proteins to be labeled. Alternatively, a fusion partner may bind to a specific sequence on the expression vector, enabling the fusion partner and associated EGFR targeting protein to be linked covalently or noncovalently with the nucleic acid that encodes them. For example, U.S. Ser. No. 09/642,574; U.S. Ser. No. 10/080,376; U.S. Ser. No. 09/792,630; U.S. Ser. No. 10/023,208; U.S. Ser. No. 09/792,626; U.S. Ser. No. 10/082,671; U.S. Ser. No. 09/953,351; U.S. Ser. No. 10/097,100; U.S. Ser. No. 60/366,658; PCT WO 00/22906; PCT WO 01/49058; PCT WO 02/04852; PCT WO 02/04853; PCT WO 02/08023; PCT WO 01/28702; and PCT WO 02/07466 describe such a fusion partner and technique that may find use in the present invention.

The methods of introducing exogenous nucleic acid into host cells are well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

In a preferred embodiment, EGFR targeting proteins are purified or isolated after expression. Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of EGFR targeting proteins. For example, the bacterial proteins A and G bind to the Fc region. Likewise, the bacterial protein L binds to the Fab region of some antibodies, as of course does the antibody's target antigen. Purification may often be enabled by a particular fusion partner. For example, EGFR targeting proteins may be purified using glutathione resin if a GST fusion is employed, Ni⁺² affinity chromatography if a His-tag is employed, or immobilized anti-flag antibody if a flag-tag is used. For general guidance in suitable purification techniques, see Protein Purification: Principles and Practice, 3^{rd} Ed., Scopes, Springer-Verlag, N.Y., 1994. The degree of purification necessary will vary depending on the screen or use of the EGFR targeting proteins. In some instances no purification is necessary. For example in one embodiment, if the EGFR targeting proteins are secreted, screening may take place directly from the media. As is well known in the art, some methods of selection do not involve purification of proteins. Thus, for example, if a library of EGFR targeting proteins is made into a phage display library, protein purification may not be performed.

Experimental Testing of EGFR Targeting Proteins

Assays

EGFR targeting proteins may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label. The use of fusion partners has been discussed above. By “labeled” herein is meant that the EGFR targeting proteins of the invention have one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and colorimetric dyes, or molecules such as biotin that enable other labeling methods. Labels may be incorporated into the compound at any position and may be incorporated in vitro or in vivo during protein expression.

In a preferred embodiment, the functional and/or biophysical properties of EGFR targeting proteins are screened in an in vitro assay. In vitro assays may allow a broad dynamic range for screening properties of interest. Properties of EGFR targeting proteins that may be screened include but are not limited to stability, solubility, and affinity for Fc ligands, for example FcγRs. Multiple properties may be screened simultaneously or individually. Proteins may be purified or unpurified, depending on the requirements of the assay. In one embodiment, the screen is a qualitative or quantitative binding assay for binding of EGFR targeting proteins to a protein or nonprotein molecule that is known or thought to bind the EGFR targeting protein. In a preferred embodiment, the screen is a binding assay for measuring binding to the EGFR target antigen. In an alternately preferred embodiment, the screen is an assay for binding of EGFR targeting proteins to an Fc ligand, including but are not limited to the family of FcγRs, the neonatal receptor FcRn, the complement protein C1q, and the bacterial proteins A and G. Said Fc ligands may be from any organism, with humans, mice, rats, rabbits, and monkeys preferred. Binding assays can be carried out using a variety of methods known in the art, including but not limited to FRET (Fluorescence Resonance Energy Transfer) and BRET (Bioluminescence Resonance Energy Transfer)-based assays, AlphaScreen™ (Amplified Luminescent Proximity Homogeneous Assay), Scintillation Proximity Assay, ELISA (Enzyme-Linked Immunosorbent Assay), SPR (Surface Plasmon Resonance, also known as BIACORE®), isothermal titration calorimetry, differential scanning calorimetry, gel electrophoresis, and chromatography including gel filtration. These and other methods may take advantage of some fusion partner or label of the EGFR targeting protein. Assays may employ a variety of detection methods including but not limited to chromogenic, fluorescent, luminescent, or isotopic labels.

The biophysical properties of EGFR targeting proteins, for example stability and solubility, may be screened using a variety of methods known in the art. Protein stability may be determined by measuring the thermodynamic equilibrium between folded and unfolded states. For example, EGFR targeting proteins of the present invention may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an EGFR targeting protein may be quantitatively or qualitatively determined using a wide range of methods that are known in the art. Methods which may find use in the present invention for characterizing the biophysical properties of EGFR targeting proteins include gel electrophoresis, isoelectric focusing, capillary electrophoresis, chromatography such as size exclusion chromatography, ion-exchange chromatography, and reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assay. Structural analysis employing X-ray crystallographic techniques and NMR spectroscopy may also find use. In one embodiment, stability and/or solubility may be measured by determining the amount of protein solution after some defined period of time. In this assay, the protein may or may not be exposed to some extreme condition, for example elevated temperature, low pH, or the presence of denaturant. Because function typically requires a stable, soluble, and/or well-folded/structured protein, the aforementioned functional and binding assays also provide ways to perform such a measurement. For example, a solution comprising an EGFR targeting protein could be assayed for its ability to bind target antigen, then exposed to elevated temperature for one or more defined periods of time, then assayed for antigen binding again. Because unfolded and aggregated protein is not expected to be capable of binding antigen, the amount of activity remaining provides a measure of the EGFR targeting protein's stability and solubility.

In a preferred embodiment, the library is screened using one or more cell-based or in vitro assays. For such assays, EGFR targeting proteins, purified or unpurified, are typically added exogenously such that cells are exposed to individual variants or groups of variants belonging to a library. These assays are typically, but not always, based on the biology of the ability of the anti-EGFR antibody or Fc fusion to bind to EGFR and mediate some biochemical event, for example effector functions like cellular lysis, phagocytosis, ligand/receptor binding inhibition, inhibition of growth and/or proliferation, apoptosis and the like. Such assays often involve monitoring the response of cells to EGFR targeting protein, for example cell survival, cell death, cellular phagocytosis, cell lysis, change in cellular morphology, or transcriptional activation such as cellular expression of a natural gene or reporter gene. For example, such assays may measure the ability of EGFR targeting proteins to elicit ADCC, ADCP, or CDC. For some assays additional cells or components, that is in addition to the target cells, may need to be added, for example serum complement, or effector cells such as peripheral blood monocytes (PBMCs), NK cells, macrophages, and the like. Such additional cells may be from any organism, preferably humans, mice, rat, rabbit, and monkey. Crosslinked or monomeric antibodies and Fc fusions may cause apoptosis of certain cell lines expressing the antibody's target antigen, or they may mediate attack on target cells by immune cells which have been added to the assay. Methods for monitoring cell death or viability are known in the art, and include the use of dyes, fluorophores, immunochemical, cytochemical, and radioactive reagents. For example, caspase assays or annexin-flourconjugates may enable apoptosis to be measured, and uptake or release of radioactive substrates (e.g. Chromium-51 release assays) or the metabolic reduction of fluorescent dyes such as alamar blue may enable cell growth, proliferation or activation to be monitored. In a preferred embodiment, the DELFIA® EuTDA-based cytotoxicity assay (Perkin Elmer, Mass.) is used. Alternatively, dead or damaged target cells may be monitored by measuring the release of one or more natural intracellular proteins, for example lactate dehydrogenase. Transcriptional activation may also serve as a method for assaying function in cell-based assays. In this case, response may be monitored by assaying for natural genes or proteins which may be up-regulated or down-regulated, for example the release of certain interleukins may be measured, or alternatively readout may be via a luciferase or GFP-reporter construct. Cell-based assays may also involve the measure of morphological changes of cells as a response to the presence of an EGFR targeting protein. Cell types for such assays may be prokaryotic or eukaryotic, and a variety of cell lines that are known in the art may be employed. Alternatively, cell-based screens are performed using cells that have been transformed or transfected with nucleic acids encoding the EGFR targeting proteins.

In vitro assays include but are not limited to binding assays, ADCC, CDC, cytotoxicity, proliferation, peroxide/ozone release, chemotaxis of effector cells, inhibition of such assays by reduced effector function antibodies; ranges of activities such as >100× improvement or >100× reduction, blends of receptor activation and the assay outcomes that are expected from such receptor profiles.

Animal Models

The biological properties of the EGFR targeting proteins of the present invention may be characterized in cell, tissue, and whole organism experiments. As is know in the art, drugs are often tested in animals, including but not limited to mice, rats, rabbits, dogs, cats, pigs, and monkeys, in order to measure a drug's efficacy for treatment against a disease or disease model, or to measure a drug's pharmacokinetics, toxicity, and other properties. Said animals may be referred to as disease models. With respect to the EGFR targeting proteins of the present invention, a particular challenge arises when using animal models to evaluate the potential for in-human efficacy of candidate polypeptides—this is due, at least in part, to the fact that EGFR targeting proteins that have a specific effect on the affinity for a human Fc receptor may not have a similar affinity effect with the orthologous animal receptor. These problems can be further exacerbated by the inevitable ambiguities associated with correct assignment of true orthologues (Mechetina et al., Immunogenetics, 2002 54:463-468), and the fact that some orthologues simply do not exist in the animal (e.g. humans possess an FcγRIIa whereas mice do not). Therapeutics are often tested in mice, including but not limited to nude mice, SCID mice, xenograft mice, and transgenic mice (including knockins and knockouts). For example, an anti-EGFR antibody or Fc fusion of the present invention that is intended as an anti-cancer therapeutic may be tested in a mouse cancer model, for example a xenograft mouse. In this method, a tumor or tumor cell line is grafted onto or injected into a mouse, and subsequently the mouse is treated with the therapeutic to determine the ability of the anti-EGFR antibody or Fc fusion to reduce or inhibit cancer growth and metastasis. An alternative approach is the use of a SCID murine model in which immune-deficient mice are injected with human PBLs, conferring a semi-functional and human immune system—with an appropriate array of human FcRs—to the mice that have subsequently been injected with antibodies or Fc-polypeptides that target injected human tumor cells. In such a model, the Fc-polypeptides that target the desired antigen (such as her2/neu on SkOV3 ovarian cancer cells) interact with human PBLs within the mice to engage tumoricidal effector functions. Such experimentation may provide meaningful data for determination of the potential of said EGFR targeting protein to be used as a therapeutic. Any organism, preferably mammals, may be used for testing. For example because of their genetic similarity to humans, monkeys can be suitable therapeutic models, and thus may be used to test the efficacy, toxicity, pharmacokinetics, or other property of the anti-EGFR antibodies and Fc fusions of the present invention. Tests of the EGFR targeting proteins of the present invention in humans are ultimately required for approval as drugs, and thus of course these experiments are contemplated. Thus the EGFR targeting proteins of the present invention may be tested in humans to determine their therapeutic efficacy, toxicity, pharmacokinetics, and/or other clinical properties.

The EGFR targeting proteins of the present invention may confer superior performance on Fc-containing therapeutics in animal models or in humans. The receptor binding profiles of such EGFR targeting proteins, as described in this specification, may, for example, be selected to increase the potency of cytotoxic drugs or to target specific effector functions or effector cells to improve the selectivity of the drug's action. Further, receptor binding profiles can be selected that may reduce some or all effector functions thereby reducing the side-effects or toxicity of such Fc-containing drug. For example, an EGFR targeting protein with reduced binding to FcγRIIa, FcγRI and FcγRIIa can be selected to eliminate most cell-mediated effector function, or an EGFR targeting protein with reduced binding to C1q may be selected to limit complement-mediated effector functions. In some contexts, such effector functions are known to have potential toxic effects, therefore eliminating them may increase the safety of the Fc-bearing drug and such improved safety may be characterized in animal models. In some contexts, such effector functions are known to mediate the desirable therapeutic activity, therefore enhancing them may increase the activity or potency of the Fc-bearing drug and such improved activity or potency may be characterized in animal models.

Optimized EGFR targeting proteins can be tested in a variety of orthotopic tumor models. These clinically relevant animal models are important in the study of pathophysiology and therapy of aggressive cancers like pancreatic, prostate and breast cancer. Immune deprived mice including, but not limited to athymic nude or SCID mice are frequently used in scoring of local and systemic tumor spread from the site of intraorgan (e.g. pancreas, prostate or mammary gland) injection of human tumor cells or fragments of donor patients.

In preferred embodiments, EGFR targeting proteins of the present invention may be assessed for efficacy in clinically relevant animal models of various human diseases. In many cases, relevant models include various transgenic animals for specific tumor antigens.

Relevant transgenic models such as those that express human Fc receptors (e.g., CD16 including the gamma chain, FcγRI, FcγRIIa/b, FcγRIIIa and others) could be used to evaluate and test EGFR targeting protein antibodies and Fc-fusions in their efficacy. The evaluation of EGFR targeting proteins by the introduction of human genes that directly or indirectly mediate effector function in mice or other rodents that may enable physiological studies of efficacy in tumor toxicity or other diseases such as autoimmune disorders and RA. Human Fc receptors such as FCγRIIIa may possess polymorphisms, such as that in position 158 V or F, which would further enable the introduction of specific combinations of human polymorphisms into rodents. The various studies involving polymorphism-specific FcγRs is not limited to this section, however encompasses all discussions and applications of FcγRs in general as specified in throughout this application. EGFR targeting proteins of the present invention may confer superior activity on Fc-containing drugs in such transgenic models, in particular variants with binding profiles optimized for human FcγRIIIa mediated activity may show superior activity in transgenic CD16 mice. Similar improvements in efficacy in mice transgenic for the other human Fc receptors, e.g. FcγRIIa, FcγRI, etc., may be observed for EGFR targeting proteins with binding profiles optimized for the respective receptors. Mice transgenic for multiple human receptors would show improved activity for EGFR targeting proteins with binding profiles optimized for the corresponding multiple receptors, for example as outlined in Table 1.

Because of the difficulties and ambiguities associated with using animal models to characterize the potential efficacy of candidate therapeutic antibodies in a human patient, some variant polypeptides of the present invention may find utility as proxies for assessing potential in-human efficacy. Such proxy molecules would preferably mimic—in the animal system—the FcR and/or complement biology of a corresponding candidate human EGFR targeting protein. This mimicry is most likely to be manifested by relative association affinities between specific EGFR targeting proteins and animal vs. human receptors. For example, if one were using a mouse model to assess the potential in-human efficacy of an EGFR targeting protein that has enhanced affinity for human FcγRIIIa, an appropriate proxy variant would have enhanced affinity for mouse FcγRIII-2 (mouse CD16-2). Alternatively if one were using a mouse model to assess the potential in-human efficacy of an EGFR targeting protein that has reduced affinity for the inhibitory human FcγRIIb, an appropriate proxy variant would have reduced affinity for mouse FcγRII. It should also be noted that the proxy EGFR targeting proteins could be created in the context of a human EGFR targeting protein, an animal EGFR targeting protein, or both.

In a preferred embodiment, the testing of EGFR targeting proteins may include study of efficacy in primates (e.g. cynomolgus monkey model) to facilitate the evaluation of depletion of specific target cells harboring EGFR antigen. Additional primate models include but not limited to that of the rhesus monkey and Fc polypeptides in therapeutic studies of autoimmune, transplantation and cancer.

Toxicity studies are performed to determine the antibody or Fc-fusion related-effects that cannot be evaluated in standard pharmacology profile or occur only after repeated administration of the agent. Most toxicity tests are performed in two species—a rodent and a non-rodent—to ensure that any unexpected adverse effects are not overlooked before new therapeutic entities are introduced into man. In general, these models may measure a variety of toxicities including genotoxicity, chronic toxicity, immunogenicity, reproductive/developmental toxicity and carcinogenicity. Included within the aforementioned parameters are standard measurement of food consumption, bodyweight, antibody formation, clinical chemistry, and macro- and microscopic examination of standard organs/tissues (e.g. cardiotoxicity). Additional parameters of measurement are injection site trauma and the measurement of neutralizing antibodies, if any. Traditionally, monoclonal antibody therapeutics, naked or conjugated, are evaluated for cross-reactivity with normal tissues, immunogenicity/antibody production, conjugate or linker toxicity and “bystander” toxicity of radiolabeled species. Nonetheless, such studies may have to be individualized to address specific concerns and following the guidance set by ICH S6 (Safety studies for biotechnological products also noted above). As such, the general principles are that the products are sufficiently well characterized and for which impurities/contaminants have been removed, that the test material is comparable throughout development, and GLP compliance.

The pharmacokinetics (PK) of the EGFR targeting proteins of the invention can be studied in a variety of animal systems, with the most relevant being non-human primates such as the cynomolgus, rhesus monkeys. Single or repeated i.v./s.c. administration(s) over a dose range of about 6000-fold (about 0.05-300 mg/kg) can be evaluated for the half-life (days to weeks) using plasma concentration and clearance as well as volume of distribution at a steady state and level of systemic absorbance can be measured. Examples of such parameters of measurement generally include maximum observed plasma concentration (Cmax), the time to reach Cmax (Tmax), the area under the plasma concentration-time curve from time 0 to infinity [AUC(0-inf] and apparent elimination half-life (T½). Additional measured parameters could include compartmental analysis of concentration-time data obtained following i.v. administration and bioavailability. Examples of pharmacological/toxicological studies using cynomolgus have been established for Rituxan® (rituxumab) and Zevalin® (ibritumomab tiuxetan) in which monoclonal antibodies to CD20 are cross-reactive. Biodistribution, dosimetry (for radiolabled antibodies or Fc fusions), and PK studies can also be done in rodent models. Such studies would evaluate tolerance at all doses administered, toxicity to local tissues, preferential localization to rodent xenograft animal models, depletion of target cells (e.g. CD20 positive cells).

The EGFR targeting proteins of the present invention confer superior pharmacokinetics on Fc-containing therapeutics in animal systems or in humans. For example, increased binding to FcRn may increase the half-life and exposure of the Fc-containing drug. Alternatively, decreased binding to FcRn may decrease the half-life and exposure of the Fc-containing drug in cases where reduced exposure is favorable such as when such drug has side-effects.

It is known in the art that the array of Fc receptors is differentially expressed on various immune cell types, as well as in different tissues. Differential tissue distribution of Fc receptors may ultimately have an impact on the pharmacodynamic (PD) and pharmacokinetic (PK) properties of EGFR targeting proteins of the present invention. Because EGFR targeting proteins of the presentation have varying affinities for the array of Fc receptors, further screening of the polypeptides for PD and/or PK properties may be extremely useful for defining the optimal balance of PD, PK, and therapeutic efficacy conferred by each candidate polypeptide.

Pharmacodynamic studies may include, but are not limited to, targeting specific tumor cells or blocking signaling mechanisms, measuring depletion of target antigen expressing cells or signals, etc. The EGFR targeting proteins of the present invention may target particular effector cell populations and thereby direct Fc-containing drugs to recruit certain activities to improve potency or to increase penetration into a particularly favorable physiological compartment. For example, neutrophil activity and localization can be targeted by an EGFR targeting protein that preferentially targets FcγRIIIb. Such pharmacodynamic effects may be demonstrated in animal models or in humans.

Clinical Use of EGFR Targeting Proteins

The EGFR targeting proteins of the present invention may be used for various therapeutic purposes. As will be appreciated by those in the art, the EGFR targeting proteins of the present invention may be used for any therapeutic purpose that antibodies, Fc fusions, and the like may be used for. In a preferred embodiment, the EGFR targeting proteins are administered to a patient to treat disorders including but not limited to autoimmune and inflammatory diseases, infectious diseases, and cancer.

A “patient” for the purposes of the present invention includes both humans and other animals, preferably mammals and most preferably humans. Thus the EGFR targeting proteins of the present invention have both human therapy and veterinary applications. The term “treatment” in the present invention is meant to include therapeutic treatment, as well as prophylactic, or suppressive measures for a disease or disorder. Thus, for example, successful administration of an EGFR targeting protein prior to onset of the disease results in treatment of the disease. As another example, successful administration of an optimized EGFR targeting protein after clinical manifestation of the disease to combat the symptoms of the disease comprises treatment of the disease. “Treatment” also encompasses administration of an optimized EGFR targeting protein after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

Diseases

In one embodiment, an EGFR targeting protein of the present invention is administered to a patient having a disease involving inappropriate expression of a protein or other molecule. Within the scope of the present invention this is meant to include diseases and disorders characterized by aberrant proteins, due for example to alterations in the amount of a protein present, protein localization, posttranslational modification, conformational state, the presence of a mutant or pathogen protein, etc. Similarly, the disease or disorder may be characterized by alterations molecules including but not limited to polysaccharides and gangliosides. An overabundance may be due to any cause, including but not limited to overexpression at the molecular level, prolonged or accumulated appearance at the site of action, or increased activity of a protein relative to normal. Included within this definition are diseases and disorders characterized by a reduction of a protein. This reduction may be due to any cause, including but not limited to reduced expression at the molecular level, shortened or reduced appearance at the site of action, mutant forms of a protein, or decreased activity of a protein relative to normal. Such an overabundance or reduction of a protein can be measured relative to normal expression, appearance, or activity of a protein, and said measurement may play an important role in the development and/or clinical testing of the EGFR targeting proteins of the present invention.

By “cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

More particular examples of such cancers include hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g., nasopharyngeal cancer, salivary gland carcinoma, and esophagael cancer), lung (e.g., small-cell lung cancer, non-small cell lung cancer, adenocarcinoma of the lung and squamous carcinoma of the lung), digestive system (e.g., gastric or stomach cancer including gastrointestinal cancer, cancer of the bile duct or biliary tract, colon cancer, rectal cancer, colorectal cancer, and anal carcinoma), reproductive system (e.g., testicular, penile, or prostate cancer, uterine, vaginal, vulval, cervical, ovarian, and endometrial cancer), skin (e.g., melanoma, basal cell carcinoma, squamous cell cancer, actinic keratosis), liver (e.g., liver cancer, hepatic carcinoma, hepatocellular cancer, and hepatoma), bone (e.g., osteoclastoma, and osteolytic bone cancers) additional tissues and organs (e.g., pancreatic cancer, bladder cancer, kidney or renal cancer, thyroid cancer, breast cancer, cancer of the peritoneum, and Kaposi's sarcoma), and tumors of the vascular system (e.g., angiosarcoma and hemagiopericytoma).

By “autoimmune diseases” herein include allogenic islet graft rejection, alopecia areata, ankylosing spondylitis, antiphospholipid syndrome, autoimmune Addison's disease, antineutrophil cytoplasmic autoantibodies (AN CA), autoimmune diseases of the adrenal gland, autoimmune hemolytic anemia, autoimmune hepatitis, autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune dysfunction syndrome, chronic inflammatory demyelinating polyneuropathy, Churg-Strauss syndrome, cicatrical pemphigoid, CREST syndrome, cold agglutinin disease, Crohn's disease, dermatomyositis, discoid lupus, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, immune mediated thrombocytopenia, juvenile arthritis, Kawasaki's disease, lichen plantus, lupus erthymatosis, Meniere's disease, mixed connective tissue disease, multiple sclerosis, type 1 diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, psoriasis, psoriatic arthritis, Reynauld's phenomenon, Reiter's syndrome, rheumatoid arthritis, sarcoidosis, scleroderma, Sjorgen's syndrome, solid organ transplant rejection, stiff-man syndrome, systemic lupus erythematosus, takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis.

By “inflammatory disorders” herein include acute respiratory distress syndrome (ARDS), acute septic arthritis, allergic encephalomyelitis, allergic rhinitis, allergic vasculitis, allergy, asthma, atherosclerosis, chronic inflammation due to chronic bacterial or viral infectionis, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy.

By “infectious diseases” herein include diseases caused by pathogens such as viruses, bacteria, fungi, protozoa, and parasites. Infectious diseases may be caused by viruses including adenovirus, cytomegalovirus, dengue, Epstein-Barr, hanta, hepatitis A, hepatitis B, hepatitis C, herpes simplex type I, herpes simplex type II, human immunodeficiency virus, (HIV), human papilloma virus (HPV), influenza, measles, mumps, papova virus, polio, respiratory syncytial virus, rinderpest, rhinovirus, rotavirus, rubella, SARS virus, smallpox, viral meningitis, and the like. Infections diseases may also be caused by bacteria including Bacillus antracis, Borrelia burgdorferi, Campylobacter jejuni, Chlamydia trachomatis, Clostridium botulinum, Clostridium tetani, Diptheria, E. coli, Legionella, Helicobacter pylori, Mycobacterium rickettsia, Mycoplasma nesisseria, Pertussis, Pseudomonas aeruginosa, S. pneumonia, Streptococcus, Staphylococcus, Vibria cholerae, Yersinia pestis, and the like. Infectious diseases may also be caused by fungi such as Aspergillus fumigatus, Blastomyces dermatitidis, Candida albicans, Coccidioides immitis, Cryptococcus neoformans, Histoplasma capsulatum, Penicillium marneffei, and the like. Infectious diseases may also be caused by protozoa and parasites such as chlamydia, kokzidioa, leishmania, malaria, rickettsia, trypanosoma, and the like.

Furthermore, EGFR targeting proteins of the present invention may be used to prevent or treat additional conditions including but not limited to heart conditions such as congestive heart failure (CHF), myocarditis and other conditions of the myocardium; skin conditions such as rosecea, acne, and eczema; bone and tooth conditions such as bone loss, osteoporosis, Paget's disease, Langerhans' cell histiocytosis, periodontal disease, disuse osteopenia, osteomalacia, monostotic fibrous dysplasia, polyostotic fibrous dysplasia, bone metastasis, bone pain management, humoral malignant hypercalcemia, periodontal reconstruction, spinal cord injury, and bone fractures; metabolic conditions such as Gaucher's disease; endocrine conditions such as Cushing's syndrome; and neurological conditions.

Formulation

Pharmaceutical compositions of the present invention may include an EGFR targeting protein of the present invention and one or more therapeutically active agents. Formulations of the EGFR targeting proteins of the present invention are prepared for storage by mixing said EGFR targeting protein having the desired degree of purity with optional pharmaceutically acceptable carriers, excipients or stabilizers (See, for example, Remington's Pharmaceutical Sciences 16th edition, Osol, A. Ed., 1980), in the form of lyophilized formulations or aqueous solutions. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™ PLURONICO® or polyethylene glycol (PEG). In a preferred embodiment, the pharmaceutical composition that comprises the EGFR targeting protein of the present invention may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration are preferrably sterile. This is readily accomplished by filtration through sterile filtration membranes or other methods.

The EGFR targeting proteins disclosed herein may also be formulated as immunoliposomes. A liposome is a small vesicle comprising various types of lipids, phospholipids and/or surfactant that is useful for delivery of a therapeutic agent to a mammal. Liposomes containing the EGFR targeting protein are prepared by methods known in the art, such as described in Epstein et al., 1985, Proc Natl Acad Sci USA, 82:3688; Hwang et al., 1980, Proc Natl Acad Sci USA, 77:4030; U.S. Pat. No. 4,485,045; U.S. Pat. No. 4,544,545; and PCT WO 97/38731. Liposomes with enhanced circulation time are disclosed in U.S. Pat. No. 5,013,556. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. A chemotherapeutic agent or other therapeutically active agent is optionally contained within the liposome (Gabizon et al., 1989, J National Cancer Inst 81:1484).

The EGFR targeting protein and other therapeutically active agents may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides (U.S. Pat. No. 3,773,919), copolymers of L-glutamic acid and gamma ethyl-L-glutamate, non-degradable ethylene-vinyl acetate, degradable lactic acid-glycolic acid copolymers such as the LUPRON DEPOT® (which are injectable microspheres composed of lactic acid-glycolic acid copolymer and leuprolide acetate), poly-D-(-)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

Administration

Administration of the pharmaceutical composition comprising an EGFR targeting protein of the present invention, preferably in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances, for example for the treatment of wounds, inflammation, etc., the EGFR targeting protein may be directly applied as a solution or spray. As is known in the art, the pharmaceutical composition may be formulated accordingly depending upon the manner of introduction or administration.

Subcutaneous administration may be preferable in some circumstances because the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate (see, for example, WO 04091658). Anti-EGFR antibodies or Fc fusions of the present invention may be more amenable to subcutaneous administration due to, for example, increased potency, improved serum half-life, or enhanced solubility.

As is known in the art, protein therapeutics are often delivered by IV infusion or bolus. The EGFR targeting proteins of the present invention may also be delivered using such methods. For example, administration may be by venous or intravenous infusion with 0.9% sodium chloride as an infusion vehicle.

Pulmonary delivery may be accomplished using an inhaler or nebulizer and a formulation comprising an aerosolizing agent. For example, AERx® inhalable technology commercially available from Aradigm, or Inhance™ pulmonary delivery system commercially available from Nektar Therapeutics may be used. EGFR targeting proteins of the present invention may be more amenable to intrapulmonary delivery. FcRn is present in the lung, and may promote transport from the lung to the bloodstream (e.g., Syntonix WO 04004798, Bitonti et.al. (2004) Proc. Nat. Acad. Sci. 101:9763-8). Accordingly, anti-EGFR antibodies or Fc fusions that bind FcRn more effectively in the lung or that are released more efficiently in the bloodstream may have improved bioavailability following intrapulmonary administration. EGFR targeting proteins of the present invention may also be more amenable to intrapulmonary administration due to, for example, improved solubility or altered isoelectric point.

Furthermore, EGFR targeting proteins of the present invention may be more amenable to oral delivery due to, for example, improved stability at gastric pH and increased resistance to proteolysis. Furthermore, FcRn appears to be expressed in the intestinal epithelia of adults (Dickinson et al. (1999) J. Clin. Invest. 104:903-11), so anti-EGFR antibodies or Fc fusions of the present invention with improved FcRn interaction profiles may show enhanced bioavailability following oral administration. FcRn mediated transport of EGFR targeting proteins may also occur at other mucus membranes such as those in the gastrointestinal, respiratory, and genital tracts (Yoshida et. al. (2004) Immunity 20:769-83).

In addition, any of a number of delivery systems are known in the art and may be used to administer the EGFR targeting proteins of the present invention. Examples include, but are not limited to, encapsulation in liposomes, microparticles, microspheres (e.g., PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the LUPRON DEPOT®, and poly-D-(−)-3-hydroxyburyric acid. It is also possible to administer a nucleic acid encoding the EGFR targeting protein of the current invention, for example by retroviral infection, direct injection, or coating with lipids, cell surface receptors, or other transfection agents. In all cases, controlled release systems may be used to release the EGFR targeting protein at or close to the desired location of action.

Dosing

The dosing amounts and frequencies of administration are, in a preferred embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the therapeutically active EGFR targeting protein in the formulation may vary from about 0.1 to 100 weight %. In a preferred embodiment, the concentration of the EGFR targeting protein is in the range of 0.003 to 1.0 molar. In order to treat a patient, a therapeutically effective dose of the EGFR targeting protein of the present invention may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.1, 1, 10, or 50 mg/kg of body weight, with 1 to 10 mg/kg being preferred.

In some embodiments, only a single dose of the EGFR targeting protein is used. In other embodiments, multiple doses of the EGFR targeting protein are administered. The elapsed time between administrations may be less than 1 hour, about 1 hour, about 1-2 hours, about 2-3 hours, about 3-4 hours, about 6 hours, about 12 hours, about 24 hours, about 48 hours, about 24 days, about 4-6 days, about 1 week, about 2 weeks, or more than 2 weeks.

In other embodiments the EGFR targeting proteins of the present invention are administered in metronomic dosing regimes, either by continuous infusion or frequent administration without extended rest periods. Such metronomic administration may involve dosing at constant intervals without rest periods. Typically such regimens encompass chronic low-dose or continuous infusion for an extended period of time, for example 1-2 days, 1-2 weeks, 1-2 months, or up to 6 months or more. The use of lower doses may minimize side effects and the need for rest periods.

In certain embodiments the EGFR targeting protein of the present invention and one or more other prophylactic or therapeutic agents are cyclically administered to the patient. Cycling therapy involves administration of a first agent at one time, a second agent at a second time, optionally additional agents at additional times, optionally a rest period, and then repeating this sequence of administration one or more times. The number of cycles is typically from 2-10. Cycling therapy may reduce the development of resistance to one or more agents, may minimize side effects, or may improve treatment efficacy.

Combination Therapies

The EGFR targeting proteins of the present invention may be administered concomitantly with one or more other therapeutic regimens or agents. The additional therapeutic regimes or agents may be used to improve the efficacy or safety of the EGFR targeting protein. Also, the additional therapeutic regimes or agents may be used to treat the same disease or a comorbidity rather than to alter the action of the EGFR targeting protein. For example, an EGFR targeting protein of the present invention may be administered to the patient along with chemotherapy, radiation therapy, or both chemotherapy and radiation therapy. The EGFR targeting protein of the present invention may be administered in combination with one or more other prophylactic or therapeutic agents, including but not limited to cytotoxic agents, chemotherapeutic agents, cytokines, growth inhibitory agents, anti-hormonal agents, kinase inhibitors, anti-angiogenic agents, cardioprotectants, immunostimulatory agents, immunosuppressive agents, agents that promote proliferation of hematological cells, angiogenesis inhibitors, protein tyrosine kinase (PTK) inhibitors, additional EGFR targeting proteins, FcγRIIb or other Fc receptor inhibitors, or other therapeutic agents.

The terms “in combination with” and “co-administration” are not limited to the administration of said prophylactic or therapeutic agents at exactly the same time. Instead, it is meant that the EGFR targeting protein of the present invention and the other agent or agents are administered in a sequence and within a time interval such that they may act together to provide a benefit that is increased versus treatment with only either the EGFR targeting protein of the present invention or the other agent or agents. It is preferred that the EGFR targeting protein and the other agent or agents act additively, and especially preferred that they act synergistically. Such molecules are suitably present in combination in amounts that are effective for the purpose intended. The skilled medical practitioner can determine empirically, or by considering the pharmacokinetics and modes of action of the agents, the appropriate dose or doses of each therapeutic agent, as well as the appropriate timings and methods of administration.

In one embodiment, the EGFR targeting proteins of the present invention are administered with one or more additional molecules comprising antibodies or Fc. The EGFR targeting proteins of the present invention may be co-administered with one or more other antibodies that have efficacy in treating the same disease or an additional comorbidity; for example two antibodies may be administered that recognize two antigens that are overexpressed in a given type of cancer, or two antigens that mediate pathogenesis of an autoimmune or infectious disease.

Examples of anti-cancer antibodies that may be co-administered include, but are not limited to, anti 17-IA cell surface antigen antibodies such as Panorex® (edrecolomab); anti-4-1BB antibodies; anti-4Dc antibodies; anti-A33 antibodies such as A33 and CDP-833; anti-α1 integrin antibodies such as natalizumab; anti-α4β7 integrin antibodies such as LDP-02; anti-αVβ1 integrin antibodies such as F-200, M-200, and SJ-749; anti-αVβ3 integrin antibodies such as abciximab, CNTO-95, Mab-17E6, and Vitaxin™; anti-complement factor 5 (C5) antibodies such as 5G1.1; anti-CA125 antibodies such as OvaRex® (oregovomab); anti-CD3 antibodies such as Nuvion® (visilizumab) and Rexomab; anti-CD4 antibodies such as IDEC-151, MDX-CD4, OKT4A; anti-CD6 antibodies such as Oncolysin B and Oncolysin CD6; anti-CD7 antibodies such as HB2; anti-CD19 antibodies such as B43, MT-103, and Oncolysin B; anti-CD20 antibodies such as 2H7, 2H7.v16, 2H7.v114, 2H7.v115, Bexxar®(tositumomab), Rituxan® (rituximab), and Zevalin® (Ibritumomab tiuxetan); anti-CD22 antibodies such as Lymphocide™ (epratuzumab); anti-CD23 antibodies such as IDEC-152; anti-CD25 antibodies such as basiliximab and Zenapax® (daclizumab); anti-CD30 antibodies such as AC10, MDX-060, and SGN-30; anti-CD33 antibodies such as Mylotarg® (gemtuzumab ozogamicin), Oncolysin M, and Smart Ml 95; anti-CD38 antibodies; anti-CD40 antibodies such as SGN-40 and toralizumab; anti-CD40L antibodies such as 5c8, Antova™, and IDEC-131; anti-CD44 antibodies such as bivatuzumab; anti-CD46 antibodies; anti-CD52 antibodies such as Campath® (alemtuzumab); anti-CD55 antibodies such as SC-1; anti-CD56 antibodies such as huN901-DM1; anti-CD64 antibodies such as MDX-33; anti-CD66e antibodies such as XR-303; anti-CD74 antibodies such as IMMU-1 10; anti-CD80 antibodies such as galiximab and IDEC-1 14; anti-CD89 antibodies such as MDX-214; anti-CD123 antibodies; anti-CD138 antibodies such as B-B4-DM1; anti-CD146 antibodies such as AA-98; anti-CD148 antibodies; anti-CEA antibodies such as cT84.66, labetuzumab, and Pentacea™; anti-CTLA-4 antibodies such as MDX-101; anti-CXCR4 antibodies; anti-EGFR antibodies such as ABX-EGF, Erbitux® (cetuximab), IMC-C225, and Merck Mab 425; anti-EpCAM antibodies such as Crucell's anti-EpCAM, ING-1, and IS-IL-2; anti-ephrin B2/EphB4 antibodies; anti-Her2 antibodies such as Herceptin®), MDX-210; anti-FAP (fibroblast activation protein) antibodies such as sibrotuzumab; anti-ferritin antibodies such as NXT-211; anti-FGF-1 antibodies; anti-FGF-3 antibodies; anti-FGF-8 antibodies; anti-FGFR antibodies, anti-fibrin antibodies; anti-G250 antibodies such as WX-G250 and Rencarex®; anti-GD2 ganglioside antibodies such as EMD-273063 and TriGem; anti-GD3 ganglioside antibodies such as BEC2, KW-2871, and mitumomab; anti-gpIIb/IIIa antibodies such as ReoPro; anti-heparinase antibodies; anti-Her2/ErbB2 antibodies such as Herceptin® (trastuzumab), MDX-210, and pertuzumab; anti-HLA antibodies such as Oncolym®, Smart 1D10; anti-HM1.24 antibodies; anti-ICAM antibodies such as ICM3; anti-IgA receptor antibodies; anti-IGF-1 antibodies such as CP-751871 and EM-164; anti-IGF-1R antibodies such as IMC-A12; anti-IL-6 antibodies such as CNTO-328 and elsilimomab; anti-IL-15 antibodies such as HuMax™-IL15; anti-KDR antibodies; anti-laminin 5 antibodies; anti-Lewis Y antigen antibodies such as Hu3S193 and IGN-311; anti-MCAM antibodies; anti-Muc1 antibodies such as BravaRex and TriAb; anti-NCAM antibodies such as ERIC-1 and ICRT; anti-PEM antigen antibodies such as Theragyn and Therex; anti-PSA antibodies; anti-PSCA antibodies such as IG8; anti-Ptk antbodies; anti-PTN antibodies; anti-RANKL antibodies such as AMG-162; anti-RLIP76 antibodies; anti-SK-1 antigen antibodies such as Monopharm C; anti-STEAP antibodies; anti-TAG72 antibodies such as CC49-SCA and MDX-220; anti-TGF-β antibodies such as CAT-152; anti-TNF-α antibodies such as CDP571, CDP870, D2E7, Humira® (adalimumab), and Remicade® (infliximab); anti-TRAIL-R1 and TRAIL-R2 antibodies; anti-VE-cadherin-2 antibodies; and anti-VLA-4 antibodies such as Antegren™. Furthermore, anti-idiotype antibodies including but not limited to the GD3 epitope antibody BEC2 and the gp72 epitope antibody 105AD7, may be used. In addition, bispecific antibodies including but not limited to the anti-CD3/CD20 antibody Bi20 may be used.

Examples of antibodies that may be co-administered to treat autoimmune or inflammatory disease, transplant rejection, GVHD, and the like include, but are not limited to, anti-α4β7 integrin antibodies such as LDP-02, anti-beta2 integrin antibodies such as LDP-01, anti-complement (C5) antibodies such as 5G1.1, anti-CD2 antibodies such as BTI-322, MEDI-507, anti-CD3 antibodies such as OKT3, SMART anti-CD3, anti-CD4 antibodies such as IDEC-151, MDX-CD4, OKT4A, anti-CD11a antibodies, anti-CD14 antibodies such as IC14, anti-CD18 antibodies, anti-CD23 antibodies such as IDEC 152, anti-CD25 antibodies such as Zenapax, anti-CD40L antibodies such as 5c8, Antova, IDEC-131, anti-CD64 antibodies such as MDX-33, anti-CD80 antibodies such as IDEC-114, anti-CD147 antibodies such as ABX-CBL, anti-E-selectin antibodies such as CDP850, anti-gpIIb/IIIa antibodies such as ReoPro®/Abcixima, anti-ICAM-3 antibodies such as ICM3, anti-ICE antibodies such as VX-740, anti-FcγR1 antibodies such as MDX-33, anti-IgE antibodies such as rhuMab-E25, anti-IL-4 antibodies such as SB-240683, anti-IL-5 antibodies such as SB-240563, SCH55700, anti-IL-8 antibodies such as ABX-IL8, anti-interferon gamma antibodies, and anti-TNFa antibodies such as CDP571, CDP870, D2E7, Infliximab, MAK-195F, anti-VLA-4 antibodies such as Antegren. Examples of other Fc-containing molecules that may be co-administered to treat autoimmune or inflammatory disease, transplant rejection, GVHD, and the like include, but are not limited to, the p75 TNF receptor/Fc fusion Enbrel® (etanercept) and Regeneron's IL-1 trap.

Examples of antibodies that may be co-administered to treat infectious diseases include, but are not limited to, anti-anthrax antibodies such as ABthrax, anti-CMV antibodies such as CytoGam and sevirumab, anti-cryptosporidium antibodies such as CryptoGAM, Sporidin-G, anti-helicobacter antibodies such as Pyloran, anti-hepatitis B antibodies such as HepeX-B, Nabi-HB, anti-HIV antibodies such as HRG-214, anti-RSV antibodies such as felvizumab, HNK-20, palivizumab, RespiGam, and anti-staphylococcus antibodies such as Aurexis, Aurograb, BSYX-A110, and SE-Mab.

Alternatively, the EGFR targeting proteins of the present invention may be co-administered or with one or more other molecules that compete for binding to one or more Fc receptors. For example, co-administering inhibitors of the inhibitory receptor FcγRIIb may result in increased effector function. Similarly, co-administering inhibitors of the activating receptors such as FcγRIIIa may minimize unwanted effector function. Fc receptor inhibitors include, but are not limited to, Fc molecules that are engineered to act as competitive inhibitors for binding to FcγRIIb FcγRIIIa, or other Fc receptors, as well as other immunoglobulins and specifically the treatment called IVIg (intravenous immunoglobulin). In one embodiment, the inhibitor is administered and allowed to act before the EGFR targeting protein is administered. An alternative way of achieving the effect of sequential dosing would be to provide an immediate release dosage form of the Fc receptor inhibitor and then a sustained release formulation of the EGFR targeting protein of the invention. The immediate release and controlled release formulations could be administered separately or be combined into one unit dosage form. Administration of an FcγRIIb inhibitor may also be used to limit unwanted immune responses, for example anti-Factor VIII antibody response following Factor VIII administration to hemophiliacs.

In one embodiment, the EGFR targeting proteins of the present invention are administered with a chemotherapeutic agent. By “chemotherapeutic agent” as used herein is meant a chemical compound useful in the treatment of cancer. Examples of chemotherapeutic agents include but are not limited to alkylating agents such as thiotepa and cyclosphosphamide (CYTOXAN™); alkyl sulfonates such as busulfan, improsulfan and piposulfan; androgens such as calusterone, dromostanolone propionate, epitiostanol, mepitiostane, testolactone; anti-adrenals such as aminoglutethimide, mitotane, trilostane; anti-androgens such as flutamide, nilutamide, bicalutamide, leuprolide, and goserelin; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti estrogens including for example tamoxifen, raloxifene, aromatase inhibiting 4(5)-imidazoles, 4-hydroxytamoxifen, trioxifene, keoxifene, LY 117018, onapristone, and toremifene (Fareston); anti-metabolites such as methotrexate and 5-fluorouracil (5-FU); folic acid analogues such as denopterin, methotrexate, pteropterin, trimetrexate; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; folic acid replenisher such as frolinic acid; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; platinum analogs such as cisplatin and carboplatin; vinblastine; platinum; proteins such as arginine deiminase and asparaginase; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; taxanes, e.g. paclitaxel (TAXOL®, Bristol-Myers Squibb Oncology, Princeton, N.J.) and docetaxel (TAXOTERE®), Rhne-Poulenc Rorer, Antony, France); topoisomerase inhibitor RFS 2000; thymidylate synthase inhibitor (such as Tomudex); additional chemotherapeutics including aceglatone; aldophosphamide glycoside; aminolevulinic acid; amsacrine; bestrabucil; bisantrene; edatraxate; defofamine; demecolcine; diaziquone; difluoromethylornithine (DMFO); elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; etoposide (VP-16); ifosfamide; mitomycin C; mitoxantrone; vincristine; vinorelbine; navelbine; novantrone; teniposide; daunomycin; aminopterin; xeloda; ibandronate; CPT-11;retinoic acid; esperamicins; capecitabine. Pharmaceutically acceptable salts, acids or derivatives of any of the above may also be used.

A chemotherapeutic or other cytotoxic agent may be administered as a prodrug. By “Prodrug” as used herein is meant a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985. The prodrugs that may find use with the present invention include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the EGFR targeting proteins of the present invention include but are not limited to any of the aforementioned chemotherapeutic agents.

A variety of other therapeutic agents may find use for administration with the EGFR targeting proteins of the present invention. In one embodiment, the EGFR targeting protein is administered with an anti-angiogenic agent. By “anti-angiogenic agent” as used herein is meant a compound that blocks, or interferes to some degree, the development of blood vessels. The anti-angiogenic factor may, for instance, be a small molecule or a protein (e.g., an antibody, Fc fusion, or cytokine) that binds to a growth factor or growth factor receptor involved in promoting angiogenesis. The preferred anti-angiogenic factor herein is an antibody that binds to Vascular Endothelial Growth Factor (VEGF). Other agents that inhibit signaling through VEGF may also be used, for example RNA-based therapeutics that reduce levels of VEGF or VEGF-R expression, VEGF-toxin fusions, Regeneron's VEGF-trap, and antibodies that bind VEGF-R. In an alternate embodiment, the EGFR targeting protein is administered with a therapeutic agent that induces or enhances adaptive immune response, for example an antibody that targets CTLA-4. Additional anti-angiogenesis agents include, but are not limited to, angiostatin (plasminogen fragment), antithrombin III, angiozyme, ABT-627, Bay 12-9566, benefin, bevacizumab, bisphosphonates, BMS-275291, cartilage-derived inhibitor (CDI), CAI, CD59 complement fragment, CEP-7055, Col 3, combretastatin A-4, endostatin (collagen XVIII fragment), farnesyl transferase inhibitors, fibronectin fragment, gro-beta, halofuginone, heparinases, heparin hexasaccharide fragment, HMV833, human chorionic gonadotropin (hCG), IM-862, interferon alpha, interferon beta, interferon gamma, interferon inducible protein 10 (IP-10), interleukin-12, kringle 5 (plasminogen fragment), marimastat, metalloproteinase inhibitors (eg. TIMPs), 2-methodyestradiol, MMI 270 (CGS 27023A), plasminogen activiator inhibitor (PAI), platelet factor-4 (PF4), prinomastat, prolactin 16 kDa fragment, proliferin-related protein (PRP), PTK 787/ZK 222594, retinoids, solimastat, squalamine, SS3304, SU5416, SU6668, SU11248, tetrahydrocortisol-S, tetrathiomolybdate, thalidomide, thrombospondin-1 (TSP-1), TNP470, transforming growth factor beta (TGF-β), vasculostatin, vasostatin (calreticulin fragment), ZS6126, and ZD6474.

In a preferred embodiment, the EGFR targeting protein is administered with a tyrosine kinase inhibitor. By “tyrosine kinase inhibitor” as used herein is meant a molecule that inhibits to some extent tyrosine kinase activity of a tyrosine kinase. Examples of such inhibitors include but are not limited to quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo(2,3-d) pyrimidines; curcumin (diferuloyl methane, 4,5-bis (4-fluoroanilino)phthalimide); tyrphostines containing nitrothiophene moieties; PD-0183805 (Warner-Lambert); antisense molecules (e.g. those that bind to ErbB-encoding nucleic acid); quinoxalines (U.S. Pat. No. 5,804,396); tryphostins (U.S. Pat. No. 5,804,396); ZD6474 (Astra Zeneca); PTK-787 (Novartis/Schering A G); pan-ErbB inhibitors such as C1-1033 (Pfizer); Affinitac (ISIS 3521; Isis/Lilly); Imatinib mesylate (STI571,Gleevec®; Novartis); PKI 166 (Novartis); GW2016 (Glaxo SmithKline); C1-1033 (Pfizer); EKB-569 (Wyeth); Semaxinib (Sugen); ZD6474 (AstraZeneca); PTK-787 (Novartis/Schering A G); INC-1 C11 (Imclone); or as described in any of the following patent publications: U.S. Pat. No. 5,804,396; PCT WO 99/09016 (American Cyanimid); PCT WO 98/43960 (American Cyanamid); PCT WO 97/38983 (Warner-Lambert); PCT WO 99/06378 (Warner-Lambert); PCT WO 99/06396 (Warner-Lambert); PCT WO 96/30347 (Pfizer, Inc); PCT WO 96/33978 (AstraZeneca); PCT WO96/3397 (AstraZeneca); PCT WO 96/33980 (AstraZeneca), gefitinib (Iressa®, ZD1839, AstraZeneca), and OSI-774 (Tarceva™, OSI Pharmaceuticals/Genentech).

In another embodiment, the EGFR targeting protein is administered with one or more immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but not limited to: non-steroidal anti-inflammatory drugs (NSAIDs) such as asprin, ibuprofed, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (eg. glucocorticoids, dexamethasone, cortisone, hydroxycortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFb, IFNa, IFNb, IFNg, IL-2, IL4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFNα, IFNβ, IFNγ, IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGFβ, TNFα, TNFβ, TNF-R1, T-cell receptor, including Enbrel® (etanercept), Humira® (adalimumab), and Remicade® (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromocryptine, cyclophosphamide, cyclosporine A, D-penicillamine, deoxyspergualin, FK506, glutaraldehyde, gold, hydroxychloroquine, leflunomide, malononitriloamides (eg. leflunomide), methotrexate, minocycline, mizoribine, mycophenolate mofetil, rapamycin, and sulfasasazine.

In an alternate embodiment, EGFR targeting proteins of the present invention are administered with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

In a preferred embodiment, cytokines or other agents that stimulate cells of the immune system are co-administered with the EGFR targeting protein of the present invention. Such a mode of treatment may enhance desired effector function. For example, agents that stimulate NK cells, including but not limited to IL-2 may be co-administered. In another embodiment, agents that stimulate macrophages, including but not limited to C5a, formyl peptides such as N-formyl-methionyl-leucyl-phenylalanine (Beigier-Bompadre et. al. (2003) Scand. J. Immunol. 57: 221-8), may be co-administered. Also, agents that stimulate neutrophils, including but not limited to G-CSF, GM-CSF, and the like may be administered. Furthermore, agents that promote migration of such immunostimulatory cytokines may be used. Also additional agents including but not limited to interferon gamma, IL-3 and IL-7 may promote one or more effector functions.

In an alternate embodiment, cytokines or other agents that inhibit effector cell function are co-administered with the EGFR targeting protein of the present invention. Such a mode of treatment may limit unwanted effector function.

In an additional embodiment, the EGFR targeting protein is administered with one or more antibiotics, including but not limited to: aminoglycoside antibiotics (eg. apramycin, arbekacin, bambermycins, butirosin, dibekacin, gentamicin, kanamycin, neomycin, netilmicin, paromomycin, ribostamycin, sisomycin, spectrinomycin), aminocyclitols (eg. sprctinomycin), amphenicol antibiotics (eg. azidamfenicol, chloramphenicol, florfrnicol, and thiamphemicol), ansamycin antibiotics (eg. rifamide and rifampin), carbapenems (eg. imipenem, meropenem, panipenem); cephalosporins (eg. cefaclor, cefadroxil, cefamandole, cefatrizine, cefazedone, cefozopran, cefpimizole, cefpiramide, cefpirome, cefprozil, cefuroxine, cefixime, cephalexin, cephradine), cephamycins (cefbuperazone, cefoxitin, cefminox, cefmetazole, and cefotetan); lincosamides (eg. clindamycin, lincomycin); macrolide (eg. azithromycin, brefeldin A, clarithromycin, erythromycin, roxithromycin, tobramycin), monobactams (eg. aztreonam, carumonam, and tigernonam); mupirocin; oxacephems (eg. flomoxef, latamoxef, and moxalactam); penicillins (eg. amdinocillin, amdinocillin pivoxil, amoxicillin, bacampicillin, bexzylpenicillinic acid, benzylpenicillin sodium, epicillin, fenbenicillin, floxacillin, penamecillin, penethamate hydriodide, penicillin o-benethamine, penicillin O, penicillin V, penicillin V benzoate, penicillin V hydrabamine, penimepicycline, and phencihicillin potassium); polypeptides (eg. bacitracin, colistin, polymixin B, teicoplanin, vancomycin); quinolones (amifloxacin, cinoxacin, ciprofloxacin, enoxacin, enrofloxacin, feroxacin, flumequine, gatifloxacin, gemifloxacin, grepafloxacin, lomefloxacin, moxifloxacin, nalidixic acid, norfloxacin, ofloxacin, oxolinic acid, pefloxacin, pipemidic acid, rosoxacin, rufloxacin, sparfloxacin, temafloxacin, tosufloxacin, and trovafloxacin); rifampin; streptogramins (eg. quinupristin, dalfopristin); sulfonamides (sulfanilamide, sulfamethoxazole); tetracyclenes (chlortetracycline, demeclocycline hydrochloride, demethylchlortetracycline, doxycycline, duramycin, minocycline, neomycin, oxytetracycline, streptomycin, tetracycline, and vancomycin).

Anti-fungal agents such as amphotericin B, ciclopirox, clotrimazole, econazole, fluconazole, flucytosine, itraconazole, ketoconazole, niconazole, nystatin, terbinafine, terconazole, and tioconazole may also be used.

Antiviral agents including protease inhibitors, reverse transcriptase inhibitors, and others, including type I interferons, viral fusion inhibitors, and neuramidase inhibitors, may also be used. Examples of antiviral agents include, but are not limited to, acyclovir, adefovir, amantadine, amprenavir, clevadine, enfuvirtide, entecavir, foscarnet, gangcyclovir, idoxuridine, indinavir, lopinavir, pleconaril, ribavirin, rimantadine, ritonavir, saquinavir, trifluridine, vidarabine, and zidovudine, may be used.

The EGFR targeting proteins of the present invention may be combined with other therapeutic regimens. For example, in one embodiment, the patient to be treated with an anti-EGFR antibody or Fc fusion of the present invention may also receive radiation therapy. Radiation therapy can be administered according to protocols commonly employed in the art and known to the skilled artisan. Such therapy includes but is not limited to cesium, iridium, iodine, or cobalt radiation. The radiation therapy may be whole body irradiation, or may be directed locally to a specific site or tissue in or on the body, such as the lung, bladder, or prostate. Typically, radiation therapy is administered in pulses over a period of time from about 1 to 2 weeks. The radiation therapy may, however, be administered over longer periods of time. For instance, radiation therapy may be administered to patients having head and neck cancer for about 6 to about 7 weeks. Optionally, the radiation therapy may be administered as a single dose or as multiple, sequential doses. The skilled medical practitioner can determine empirically the appropriate dose or doses of radiation therapy useful herein. In accordance with another embodiment of the invention, the EGFR targeting protein of the present invention and one or more other anti-cancer therapies are employed to treat cancer cells ex vivo. It is contemplated that such ex vivo treatment may be useful in bone marrow transplantation and particularly, autologous bone marrow transplantation. For instance, treatment of cells or tissue(s) containing cancer cells with EGFR targeting protein and one or more other anti-cancer therapies, such as described above, can be employed to deplete or substantially deplete the cancer cells prior to transplantation in a recipient patient.

Radiation therapy may also comprise treatment with an isotopically labeled molecule, such as an antibody. Examples of radioimmunotherapeutics include but Zevalin™ (Y-90 labeled anti-CD20), LymphoCide™ (Y-90 labeled anti-CD22) and Bexxar™ (1-131 labeled anti-CD20)

It is of course contemplated that the EGFR targeting proteins of the invention may employ in combination with still other therapeutic techniques such as surgery or phototherapy.

A number of the receptors that may interact with the EGFR targeting proteins of the present invention are polymorphic in the human population. For a given patient or population of patients, the efficacy of the EGFR targeting proteins of the present invention may be affected by the presence or absence of specific polymorphisms in proteins. For example, FcγRIIIa is polymorphic at position 158, which is commonly either V (high affinity) or F (low affinity). Patients with the V/V homozygous genotype are observed to have a better clinical response to treatment with the anti-CD20 antibody Rituxan® (rituximab), likely because these patients mount a stronger NK response (Dall'Ozzo et. al. (2004) Cancer Res. 64:4664-9). Additional polymorphisms include but are not limited to FcγRIIa R131 or H131, and such polymorphisms are known to either increase or decrease Fc binding and subsequent biological activity, depending on the polymorphism. EGFR targeting proteins of the present invention may bind preferentially to a particular polymorphic form of a receptor, for example FcγRIIIa 158 V, or to bind with equivalent affinity to all of the polymorphisms at a particular position in the receptor, for example both the 158V and 158F polymorphisms of FcγRIIIa. In a preferred embodiment, EGFR targeting proteins of the present invention may have equivalent binding to polymorphisms may be used in an antibody to eliminate the differential efficacy seen in patients with different polymorphisms. Such a property may give greater consistency in therapeutic response and reduce non-responding patient populations. Such variant Fc with identical binding to receptor polymorphisms may have increased biological activity, such as ADCC, CDC or circulating half-life, or alternatively decreased activity, via modulation of the binding to the relevant Fc receptors. In a preferred embodiment, EGFR targeting proteins of the present invention may bind with higher or lower affinity to one of the polymorphisms of a receptor, either accentuating the existing difference in binding or reversing the difference. Such a property may allow creation of therapeutics particularly tailored for efficacy with a patient population possessing such polymorphism. For example, a patient population possessing a polymorphism with a higher affinity for an inhibitory receptor such as FcγRIIb could receive a drug containing an EGFR targeting protein with reduced binding to such polymorphic form of the receptor, creating a more efficacious drug.

In a preferred embodiment, patients are screened for one or more polymorphisms in order to predict the efficacy of the EGFR targeting proteins of the present invention. This information may be used, for example, to select patients to include or exclude from clinical trials or, post-approval, to provide guidance to physicians and patients regarding appropriate dosages and treatment options. For example, in patients that are homozygous or heterozygous for FcγRIIIa 158F antibody drugs, such as the anti-CD20 mAb Rituximab, are minimally effective (Carton 2002 Blood 99: 754-758; Weng 2003 J. Clin. Oncol. 21:3940-3947); such patients may show a much better clinical response to the antibodies of the present invention. In one embodiment, patients are selected for inclusion in clinical trials for an antibody of the present invention if their genotype indicates that they are likely to respond significantly better to an antibody of the present invention as compared to one or more currently used antibody therapeutics. In another embodiment, appropriate dosages and treatment regimens are determined using such genotype information. In another embodiment, patients are selected for inclusion in a clinical trial or for receipt of therapy post-approval based on their polymorphism genotype, where such therapy contains an EGFR targeting protein engineered to be specifically efficacious for such population, or alternatively where such therapy contains an EGFR targeting protein that does not show differential activity to the different forms of the polymorphism.

Included in the present invention are diagnostic tests to identify patients who are likely to show a favorable clinical response to an EGFR targeting protein of the present invention, or who are likely to exhibit a significantly better response when treated with an EGFR targeting protein of the present invention versus one or more currently used antibody therapeutics. Any of a number of methods for determining FcγR polymorphisms in humans known in the art may be used.

Furthermore, the present invention comprises prognostic tests performed on clinical samples such as blood and tissue samples. Such tests may assay for effector function activity, including but not limited to ADCC, CDC, phagocytosis, and opsonization, or for killing, regardless of mechanism, of cancerous or otherwise pathogenic cells. In a preferred embodiment, ADCC assays, such as those described previously, are used to predict, for a specific patient, the efficacy of a given EGFR targeting protein of the present invention. Such information may be used to identify patients for inclusion or exclusion in clinical trials, or to inform decisions regarding appropriate dosages and treatment regains. Such information may also be used to select a drug that contains a particular EGFR targeting protein that shows superior activity in such assay.

### EXAMPLES

Examples are provided below to illustrate the present invention. These examples are not meant to constrain the present invention to any particular application or theory of operation. For reference to immunoglobulin constant regions, positions are numbered according to the EU index as in Kabat (Kabat et al., 1991, Sequences of Proteins of Immunological Interest, 5th Ed., United States Public Health Service, National Institutes of Health, Bethesda). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

### Example 1

### Anti-EGFR Antibodies with Enhanced Effector Function

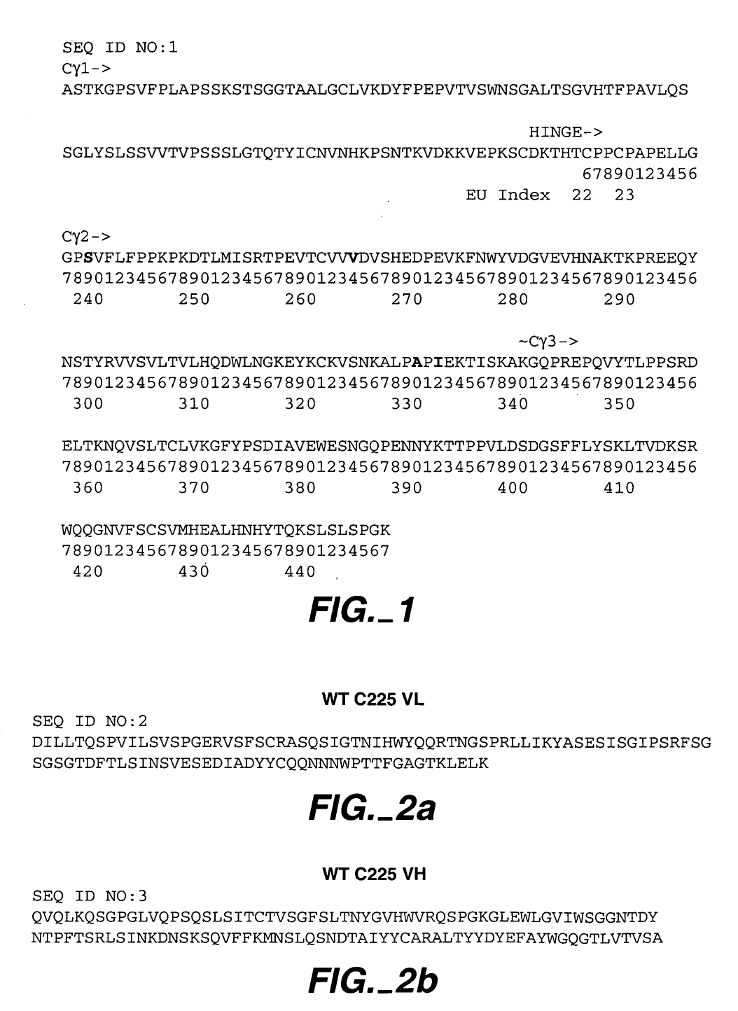

Antibodies are the most commonly used class of therapeutic proteins. As discussed, a number of favorable properties are imparted on antibodies by the Fc region, including but not limited to favorable pharmacokinetics and effector function. The latter property is particularly relevant for anti-cancer antibodies, and thus is an important property for antibodies that target EGFR. As has been discussed above and described more fully in U.S. Ser. No. 10/672,280; PCT US03/30249; U.S. Ser. No. 10/822,231; U.S. Ser. Nos. 60/568,440, 60/627,026, 60/626,991 and 60/627,774, amino acid modifications have been engineered that provide antibodies with enhanced effector function. A number of amino acid substitutions obtained in these studies, including but not limited to S239D, V2641, A330L, I332E, and combinations thereof, provide optimal enhancements in binding to FcγRs and substantially enhanced ADCC. FIG. 1 presents the amino acid sequence of constant region of human IgG1, the most frequently used antibody isotype for therapeutic purposes, with positions S239, V264, A330, and I332 highlighted.

To investigate the potential for improving the effector function of a protein that targets EGFR, a number of such Fc variants were engineered into the context of C225 (U.S. Pat. No. 4,943,533; PCT WO 96/40210), a chimeric anti-EGFR antibody that comprises the variable regions of a mouse antibody and the constant regions of human CLκ and IgG1. FIGS. 2a and 2b show the light and heavy chain variable region sequences respectively of the parent C225 antibody used in the present study. The genes for the variable regions of C225 were constructed using recursive PCR, and subcloned into the mammalian expression vector pcDNA3.1Zeo (Invitrogen) comprising the full length light kappa (CLκ) and heavy chain IgG1 constant regions. Fc variants V264I/I332E, S239D/I332E, and S239D/A330L/I332E were introduced into the C225 heavy chain using quick-change mutagenesis techniques (Stratagene). Fc variants were sequenced to confirm the fidelity of the sequence. Plasmids containing heavy chain gene (VH-CH1-CH2-CH3) (wild-type or variants) were co-transfected with plasmid containing light chain gene (VL-CLκ) into 293T cells. Media were harvested 5 days after transfection, and antibodies were purified from the supernatant using protein A affinity chromatography (Pierce, Catalog #20334).

In order to screen for FcγR binding, the extracellular region of human V158 FcγRIIIa was expressed and purified. The extracellular region of this receptor was obtained by PCR from a clone obtained from the Mammalian Gene Collection (MGC:22630). The receptor was fused with glutathione S-Transferase (GST) to enable screening. Tagged FcγRIIIa was transfected in 293T cells, and media containing secreted FcγRIIIa were harvested 3 days later and purified.

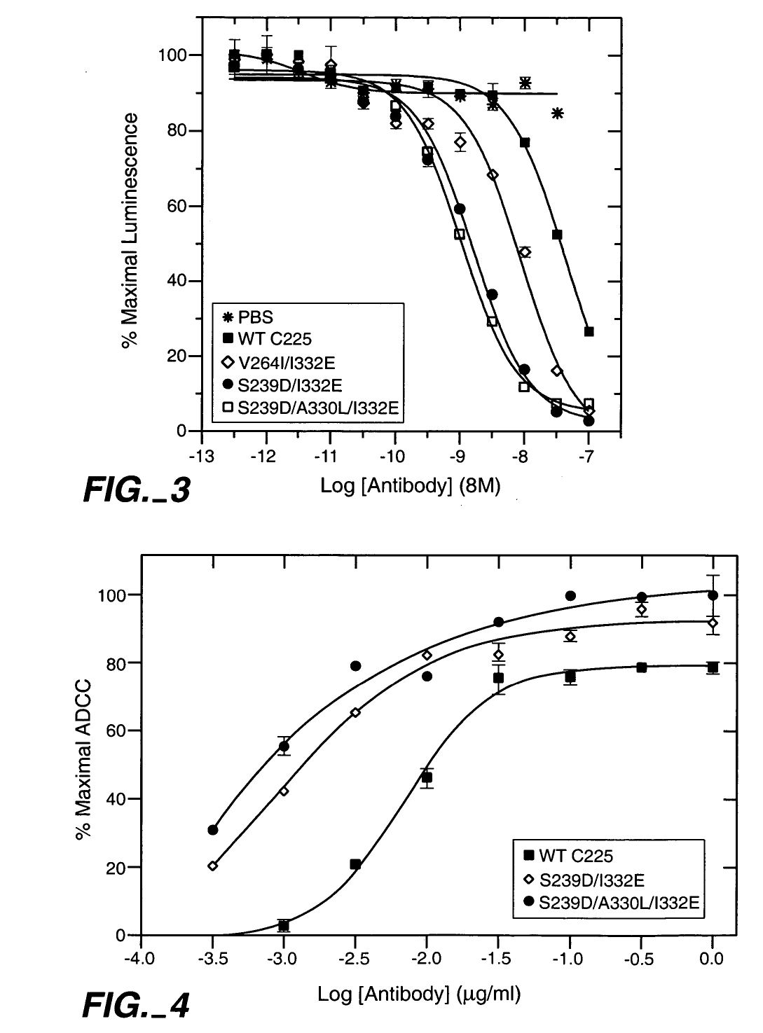

Binding affinity to human FcγRIIIa by the anti-EGFR antibodies was measured using a quantitative and extremely sensitive method, AlphaScreen™ assay. The AlphaScreen™ assay is a bead-based non-radioactive luminescent proximity assay. Laser excitation of a donor bead excites oxygen, which if sufficiently close to the acceptor bead will generate a cascade of chemiluminescent events, ultimately leading to fluorescence emission at 520-620 nm. The AlphaScreen™ assay was applied as a competition assay for screening the antibodies. Wild-type IgG1 C225 antibody was biotinylated by standard methods for attachment to streptavidin donor beads, and tagged FcγRIIIa was bound to glutathione chelate acceptor beads. In the absence of competing Fc polypeptides, wild-type antibody and FcγR interact and produce a signal at 520-620 nm. Addition of untagged antibody competes with wild-type Fc/FcγR interaction, reducing fluorescence quantitatively to enable determination of relative binding affinities. FIG. 3 shows AlphaScreen™ data for the binding of WT and Fc variant C225 antibodies to human V158 FcγRIIIa. As can be seen, the V264I/I332E, S239D/I332E, and S239D/A330L/I332E Fc variants provide substantial enhancements to the binding affinity of C225 for FcγRIIIa.

To investigate the capacity of the C225 variants to mediate effector function, a cell-based ADCC assay was carried out. Human peripheral blood monocytes (PBMCs) were isolated from buffy-coat and used as effector cells, and A431 epidermoid carcinoma cells were used as target cells. The A431 cell line expresses approximately 2.6×10⁶ copies of EGFR per cell. Target cells were incubated with varying concentration of antibodies and PBMCs at a 10:1 effector:target cell ratio, overnight at 37° C. Lysis was monitored by measuring LDH activity using the Cytotoxicity Detection Kit (LDH, Roche Diagnostic Corporation, Indianapolis, Ind.). Samples were run in triplicate to provide error estimates (n=3, ±S.D.). FIG. 4 provides the dose dependence of ADCC at various antibody concentrations. Substantial ADCC enhancements are provided by the S239D/I332E and S239D/A330L/I332E modifications relative to the WT C225 antibody. The graphs show that the antibodies differ not only in their EC50, reflecting their relative potency, but also in the maximal level of ADCC attainable by the antibodies at saturating concentrations, reflecting their relative efficacy. These two terms, potency and efficacy, are sometimes used loosely to refer to desired clinical properties. In the current experimental context, however, they are denoted as specific quantities, and therefore are here explicitly defined. By “potency” as used in the current experimental context is meant the EC50 of an EGFR targeting protein. By “efficacy” as used in the current experimental context is meant the maximal possible effector function of an EGFR targeting protein at saturating levels. FIG. 4 indicates that the Fc variants provide approximately 10- to 100-fold enhancements in potency and approximately 30% enhancements in efficacy over WT C225.

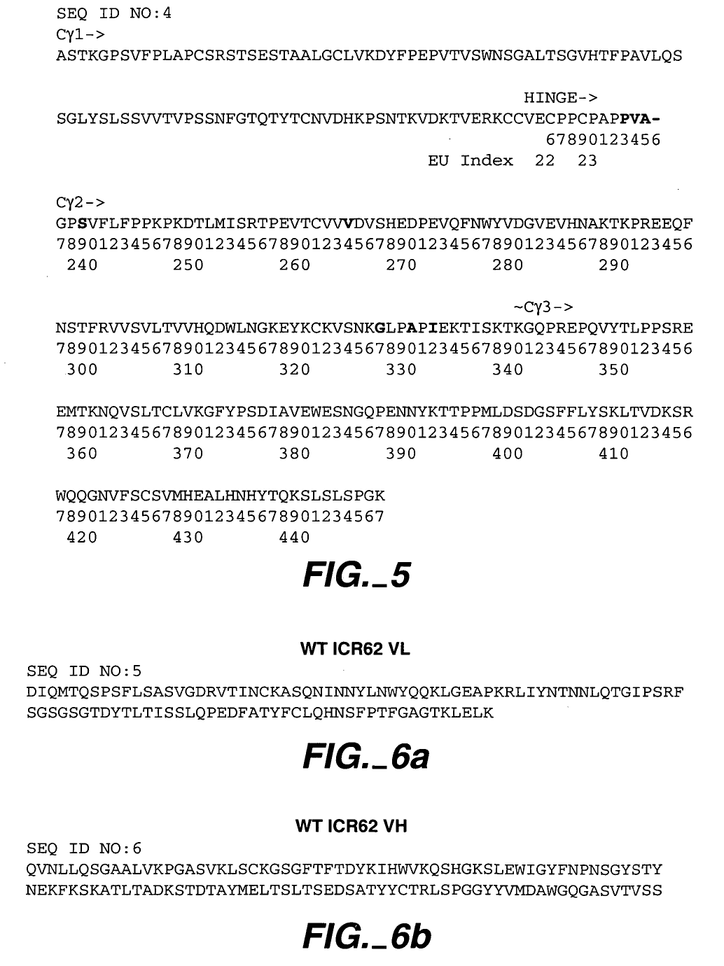

Although human IgG1 is the most commonly used constant region for therapeutic antibodies, other embodiments may utilize constant regions or variants thereof of other IgG immunoglobulin chains. Effector functions such as ADCC, ADCP, CDC, and serum half-life differ significantly between the different classes of antibodies, including for example human IgG1, IgG2, IgG3, IgG4, IgA1, IgA2, IgD, IgE, IgG, and IgM (Michaelsen et al., 1992, Molecular Immunology, 29(3): 319-326). A number of studies have explored IgG1, IgG2, IgG3, and IgG4 variants in order to investigate the determinants of the effector function differences between them. See for example Canfield & Morrison, 1991, J. Exp. Med. 173: 1483-1491; Chappel et al., 1991, Proc. Natl. Acad. Sci. USA 88(20): 9036-9040; Chappel et al., 1993, Journal of Biological Chemistry 268:25124-25131; Tao et al., 1991, J. Exp. Med. 173: 1025-1028; Tao et al., 1993, J. Exp. Med. 178: 661-667; Redpath et al., 1998, Human Immunology, 59, 720-727. As described above, it is possible to determine corresponding or equivalent residues in proteins that have significant sequence or structural homology with each other. By the same token, it is possible to use such methods to engineer amino acid modifications in an antibody or Fc fusion that comprise constant regions from other immunoglobulin classes, for example as described in U.S. Ser. Nos. 60/621,387 and 60/629,068, to provide optimal properties. As an example, the relatively poor effector function of IgG2 may be improved by replacing key FcγR binding residues with the corresponding amino acids in an IgG with better effector function, for example IgG1. FIG. 5 provides the constant region sequence of human IgG2, highlighting key residue differences between IgG2 and IgG1 with respect to FcγR binding. These residues include P233, V234, A235, −236, and G327; here −236 indicates a deletion in IgG2 relative to IgG1. One or more amino acid modifications in the parent IgG2 wherein one or more of these residues is replaced with the corresponding IgG1 amino acids, P233E, V234L, A235L, -236G, and G327A, may provide enhanced effector function. Furthermore, one or more additional amino acid modifications, for example the S239D, V264I, A330L, I332E, or combinations thereof as described above, may provide enhanced FcγR binding and effector function relative to the parent IgG2.

### Example 2

### Anti-EGFR Antibodies with Reduced Immunogenicity

The C225 variable region utilized in Example 1 is derived from a murine antibody. Indeed due to the wide use of hybridoma technology, a substantial number of antibodies are derived from nonhuman sources, for example rodent. However, nonhuman proteins are often immunogenic when administered to humans, thereby greatly reducing their therapeutic utility. Immunogenicity is the result of a complex series of responses to a substance that is perceived as foreign, and may include production of neutralizing and non-neutralizing antibodies, formation of immune complexes, complement activation, mast cell activation, inflammation, hypersensitivity responses, and anaphylaxis. Several factors can contribute to protein immunogenicity, including but not limited to protein sequence, route and frequency of administration, and patient population. Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. Thus in a preferred embodiment, protein engineering is used to reduce the immunogenicity of the EGFR targeting proteins of the present invention.

In order to reduce the potential for immunogenicity of the anti-EGFR proteins of the present invention, the immunogenicity of two anti-EGFR antibodies was reduced using a method described in U.S. Ser. Nos. 60/527,167; 60/582,613; 60/619,483; and U.S. Ser. No. 10/______, entitled “Methods of Generating Variant Proteins with Increased Host String Content and Compositions Thereof”, filed on Dec. 6, 2004. The two antibodies are C225, described above, and ICR62, a rat anti-EGFR antibody that has also been investigated clinically for the treatment of cancer (Institute of Cancer Research) (PCT WO 95/20045; Modjtahedi et al., 1993, J. Cell Biophys. 1993, 22(1-3): 129-46; Modjtahedi et al., 1993, Br J Cancer. 1993, 67(2): 247-53; Modjtahedi et al, 1996, Br J Cancer, 73(2):228-35; Modjtahedi et al, 2003, Int J Cancer, 105(2):273-80). FIG. 6 provides the light and heavy chain variable region sequences respectively of the parent chimeric [CR62 used in the present study. The genes for the C225 and ICR62 variable were constructed as described above, and subcloned into a modified pASK84 vector (Skerra, 1994, Gene 141: 79-84) comprising mouse constant regions for expression as Fabs.

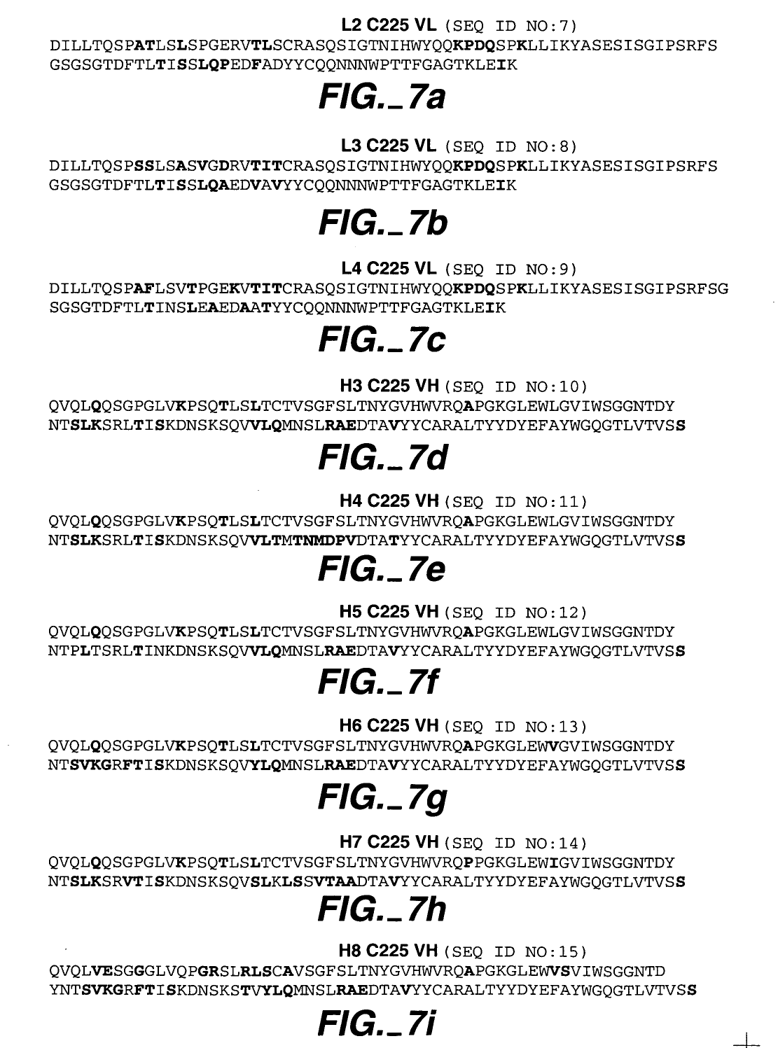

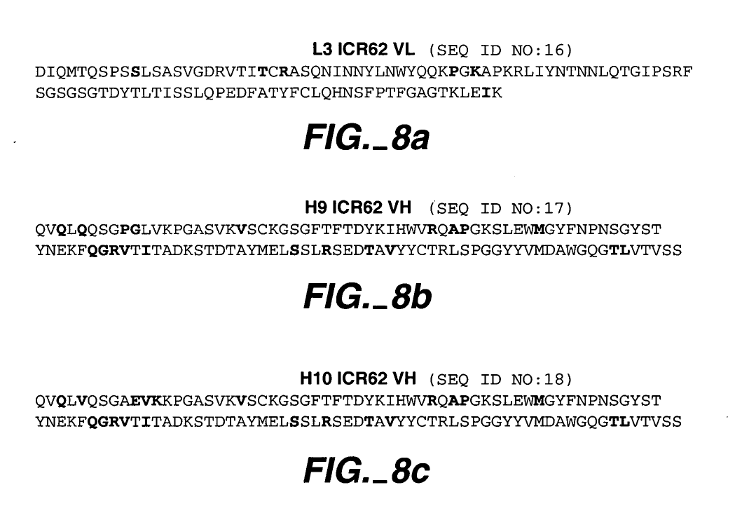

Structural models of the murine C225 and rat ICR62 variable regions were constructed using standard antibody modeling methods known in the art. Variants with reduced immunogenicity were generated by applying a human string optimization algorithm on the WT C225 and ICR62 VL and VH sequences U.S. Ser. Nos. 60/527,167; 60/582,613; 60/619,483; and U.S. Ser. No. 10/______, entitled “Methods of Generating Variant Proteins with Increased Host String Content and Compositions Thereof”, filed on Dec. 6, 2004. This algorithm heuristically samples multiple amino acid mutations that exist in the diversity of the human VL and VH germline sequences, and calculates the human string content (HSC), using a window size w=9. In these calculations, residues close to a CDR or to the VL/VH interface were masked, that is were not allowed to mutate. These calculations were run for C225 and ICR62 VL and VH in 100 separate iterations, generating a set of diverse anti-EGFR variants with greater human string content than WT. In addition to the HSC score, each sequence was evaluated for its structural and functional integrity using a nearest neighbor structure-based scoring method (see, for example, U.S. Ser. Nos. 60/528,229 and 60/602,566). Two measures of structural fitness, referred to as “Structural Consensus” and “Structural Precedence”, were also used to evaluate the variants. The output sequences were clustered based on their mutational distance from the other sequences in the set, and from these clustered output sequences were chosen a set of C225 and ICR62 VL and VH variants with reduced immunogenicity. In some cases, further substitutions were made to output sequences, using HSC and structural scores, as well as visual inspection of the modeled C225 and ICR62 structures, to evaluate fitness. FIGS. 7 and 8 present the sequences for each of the C225 and ICR62 variants with reduced immunogenicity.

Tables 2 through 5 present the human string and structural fitness scores, as well as the number of mutations relative to WT for the C225 VL and VH variants (Tables 2-3 respectively) and ICR62 VL and VH variants (Tables 4-5 respectively), as compared the corresponding WT sequences. Structural Consensus and Structural Precedence reflect the overall structural fitness of the sequences using a nearest neighbor structural approach, and Human String Content and Human String Similarity reflect the level of immunogenicity relative to an aligned set of human sequences (U.S. Ser. Nos. 60/527,167; 60/582,613; 60/619,483, filed Oct. 14, 2004 and U.S. Ser. No. 10/______, entitled “Methods of Generating Variant Proteins with Increased Host String Content and Compositions Thereof”, filed on Dec. 6, 2004; U.S. Ser. Nos. 60/528,229 and 60/602,566). In addition, the maximum identity match to the germline for each epitope in the sequences was also determined, referred to as N₉max (U.S. Ser. Nos. 60/527,167; 60/582,613; 60/619,483 and U.S. Ser. No. 10/______, entitled ” Methods of Generating Variant Proteins with Increased Host String Content and Compositions Thereof”, filed on Dec. 6, 2004). This represents the total number of strings in each sequence whose maximum identity to the corresponding strings in the human germline is 9; for w=9 this represents a perfect match, and thus this value is an additional measure of immunogenicity relative to the human sequences. Together, these parameters for the C225 and ICR62 variant sequences indicate that the variants provide substantially reduced immunogenicity relative to WT, while maintaining and in some cases improving the structural fitness of the proteins.

TABLE 2
C225 VL Variants

| | WT | L2 | L3 | L4 |
|---|---|---|---|---|
| Mutations | | 17 | 21 | 18 |
| Structural | 0.49 | 0.56 | 0.58 | 0.54 |
| Consensus | | | | |
| Structural | 0.53 | 0.57 | 0.59 | 0.57 |
| Precedence | | | | |
| Human String | 0.79 | 0.91 | 0.92 | 0.91 |
| Content | | | | |
| Human String | 0.15 | 0.51 | 0.58 | 0.57 |
| Similarity | | | | |
| N₉max | 13 | 52 | 60 | 58 |

TABLE 3

| | C225 VH Variants | | | | | | |
|---|---|---|---|---|---|---|---|
| | WT | H3 | H4 | H5 | H6 | H7 | H8 |
| Mutations | | 18 | 21 | 15 | 21 | 22 | 28 |
| Structural | 0.44 | 0.51 | 0.46 | 0.49 | 0.52 | 0.49 | 0.53 |
| Consensus | | | | | | | |
| Structural | 0.55 | 0.55 | 0.54 | 0.51 | 0.55 | 0.58 | 0.55 |
| Precedence | | | | | | | |
| Human String | 0.67 | 0.79 | 0.81 | 0.77 | 0.79 | 0.79 | 0.79 |
| Content | | | | | | | |
| Human String | 0.04 | 0.36 | 0.41 | 0.33 | 0.36 | 0.35 | 0.33 |
| Similarity | | | | | | | |
| N₉max | 3 | 42 | 48 | 38 | 42 | 41 | 39 |

TABLE 4
ICR62 VL Variants

| | WT | L2 |
|---|---|---|
| Mutations | 0 | 6 |
| Structural | 0.56 | 0.61 |
| Consensus | | |
| Structural | 0.52 | 0.57 |
| Precedence | | |
| Human String | 0.86 | 0.90 |
| Content | | |
| Human String | 0.38 | 0.56 |
| Similarity | | |
| N₉max | 37 | 56 |

TABLE 5
ICR62 VH Variants

| | WT | H9 | H10 |
|---|---|---|---|
| Mutations | 0 | 20 | 21 |
| Structural | 0.43 | 0.46 | 0.45 |
| Consensus | | | |
| Structural | 0.42 | 0.47 | 0.49 |
| Precedence | | | |
| Human String | 0.64 | 0.79 | 0.79 |
| Content | | | |
| Human String | 0.01 | 0.28 | 0.33 |
| Similarity | | | |
| N₉max | 1 | 33 | 39 |

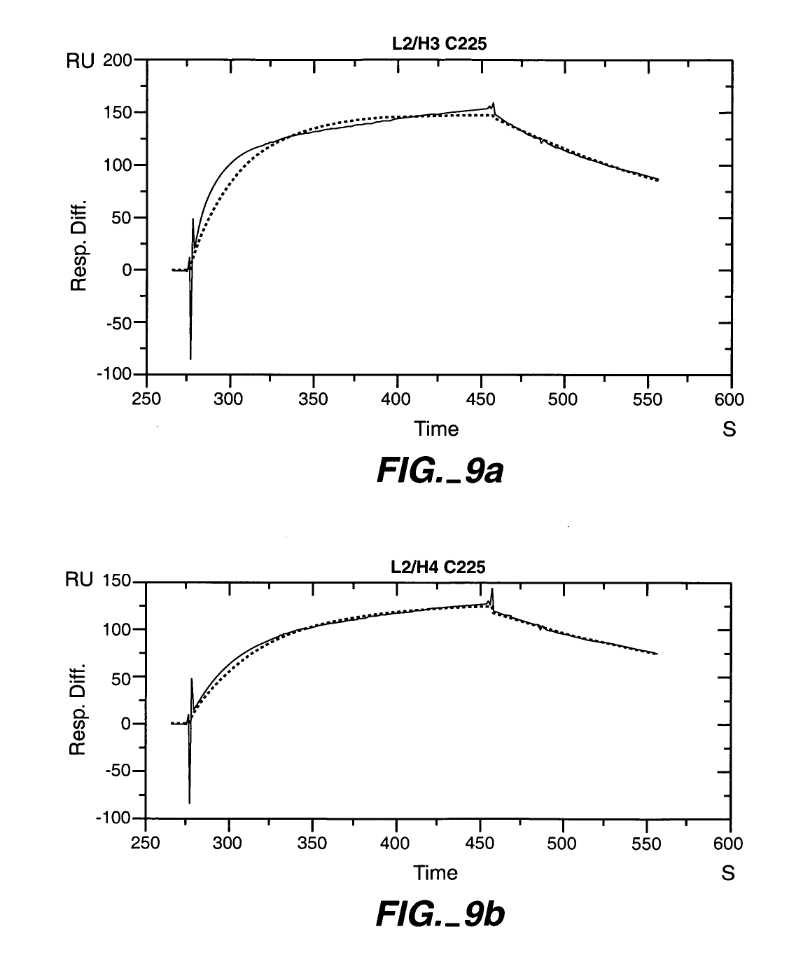

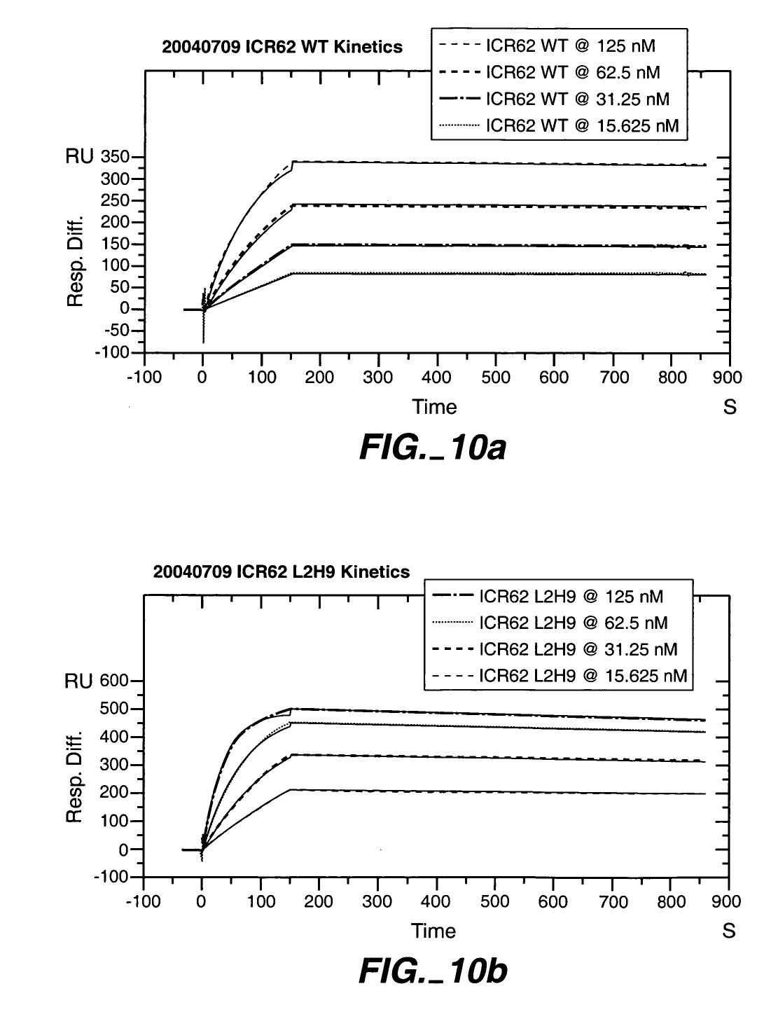

Select C225 and ICR62 variants were experimentally tested for their capacity to bind EGFR antigen. L2/H3 and L2/H4 C225 Fabs, and WT and L2/H9 ICR62 Fabs were expressed from the pASK84 vector in E. Coli with a His-tag, and purified using Nickel-affinity chromatography. Here L2/H3 C225 refers to the L2 C225 VL paired with H3 C225 heavy chain VH as described above. Antigen affinity of the C225 and ICR62 was tested using Surface Plasmon Resonance (SPR) (Biacore, Uppsala, Sweden). SPR is a sensitive and quantitative method that allows for the measurement of binding affinities of protein-protein interactions. EGFR extracellular domain (purchased commercially from R&D Systems) was covalently coupled to the dextrane matrix of a CM5 chip using NHS-linkage chemistry. C225 and ICR62 Fabs were reacted with the EGFR sensor chip surface at varying concentrations. The resulting sensorgrams for select C225 and ICR62 variants are shown in FIGS. 9 and 10. Global Langmuir fits were been carried out for the concentrations series using the BiaEvaluation curve fitting software. The on-rate constant (ka), off-rate constant (kd), equilibrium binding constant (KD=kd/ka), and predicted saturation binding signal (Rmax) derived from these fits are presented in Tables 6 and 7, along with the Chi2 which quantifies the average deviation of the fit curve from the actual data curve. The data indicate that both the C225 and ICR62 variants bind EGFR antigen, and further that the L2/H9 ICR62 variant binds EGFR antigen with comparable affinity as WT ICR62.

TABLE 6
SPR data on C225 Variants

| C225 | ka (1/Ms) | kd (1/s) | KD (M) | Rmax (RU) | Chi2 |
|---|---|---|---|---|---|
| L2/H3 | 2.79 × 10⁴ | 5.35 × 10⁻³ | 1.92 × 10⁻⁷ | 174 | 8.83 |
| L2/H4 | 1.79 × 10⁴ | 4.73 × 10⁻³ | 2.64 × 10⁻⁷ | 153 | 2.69 |

TABLE 7
SPR data on ICR62 Variants

| ICR62 | ka (1/Ms) | kd (1/s) | KD (M) | Rmax (RU) | Chi2 |
|---|---|---|---|---|---|
| WT | 9.86 × 10⁴ | 2.53 × 10⁻⁵ | 2.57 × 10⁻¹⁰ | 402 | 1.86 |
| L2/H9 | 2.35 × 10⁵ | 1.06 × 10⁻⁴ | 4.50 × 10⁻¹⁰ | 508 | 4.91 |

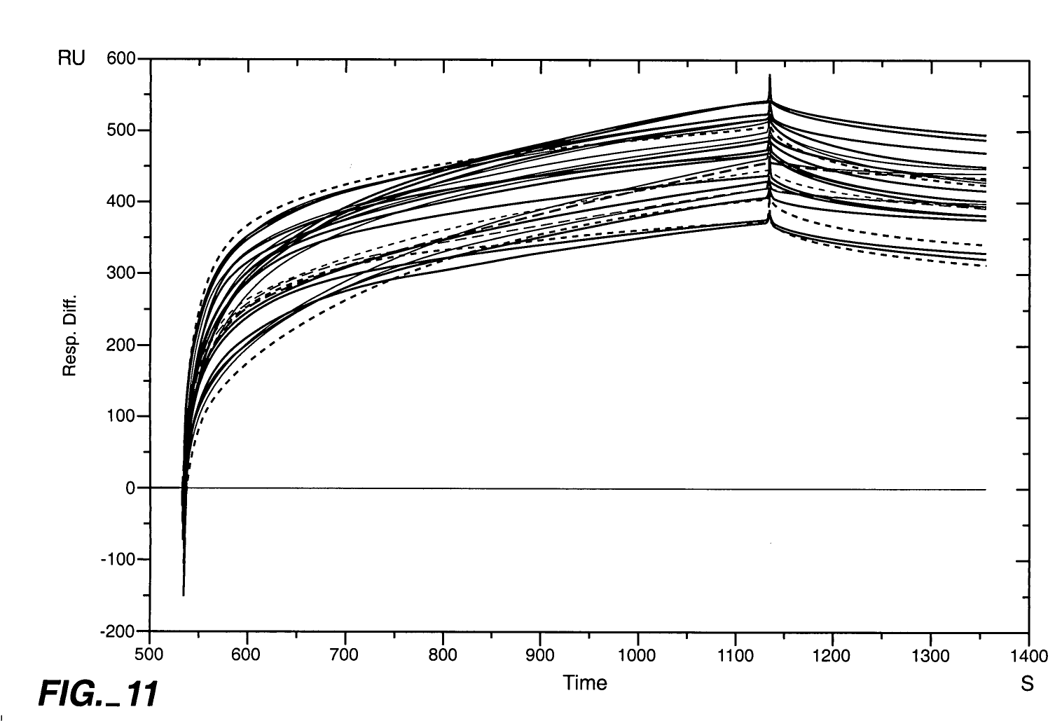

In order to investigate the anti-EGFR variants in the context of a full length antibody, the C225 WT (L0 ad H0) and variant (L2, L3, L4, H3, H4, H5, H6, H7, and H8) regions were subcloned into the mammalian expression vector pcDNA3.1Zeo (Invitrogen) as described above. All combinations of the light and heavy chain plasmids were co-transfected into 293T cells, and antibodies were expressed, harvested, and purified as described above. Binding of the C225 WT (L0/H0) and variant (L0/H3, L0/H4, L0/H5, L0/H6, L0/H7, L0/H8, L2/H3, L2/H4, L2/H5, L2/H6, L2/H7, L2/H8, L3/H3, L3/H4, L3/H5, L3/H6, L3/H7, L3/H8, L4/H3, L4/H4, L4/H5, L4/H6, L4/H7, and L4/H8) antibodies was determined using SPR similar to as described above. Full length antibodies were flowed over the EGFR sensor chip described above. FIG. 11 shows the SPR sensorgrams obtained from the experiments. The curves consist of a association phase and dissociation phase, the separation being marked by a little spike on each curve. As a very rough approximation the signal level reached near the end of the association phase can be used as an indicator for relative binding. For all the curves this signal level is within 25% of the average level indicating that none of the antibody variants have significantly lost their ability to bind to EGFR.

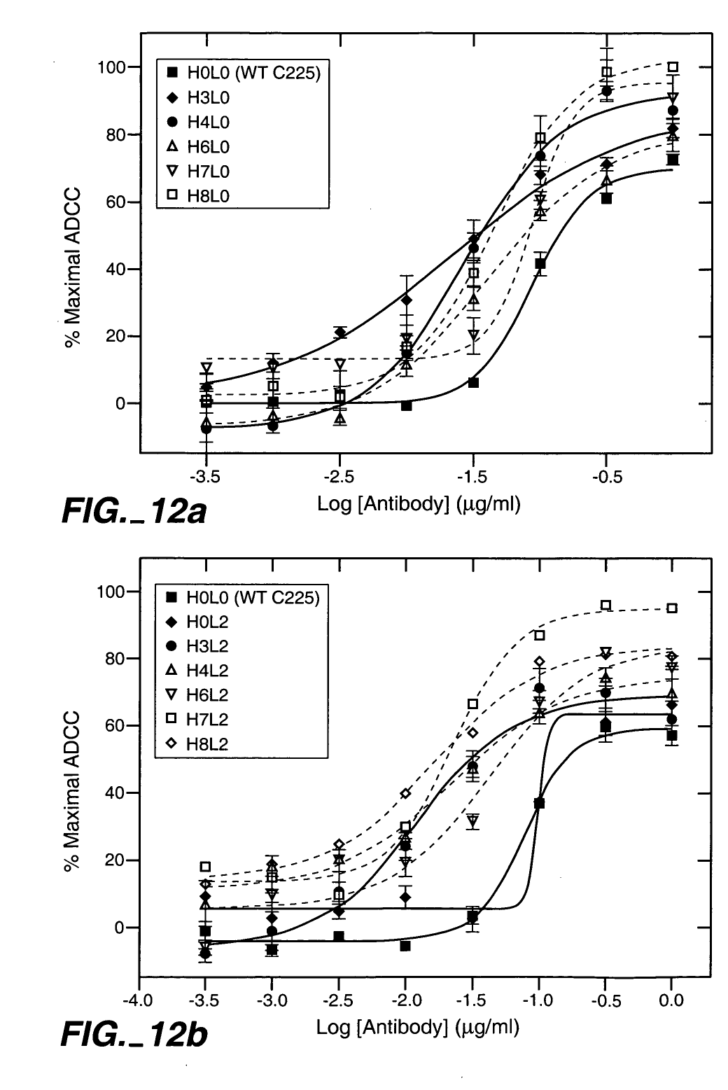

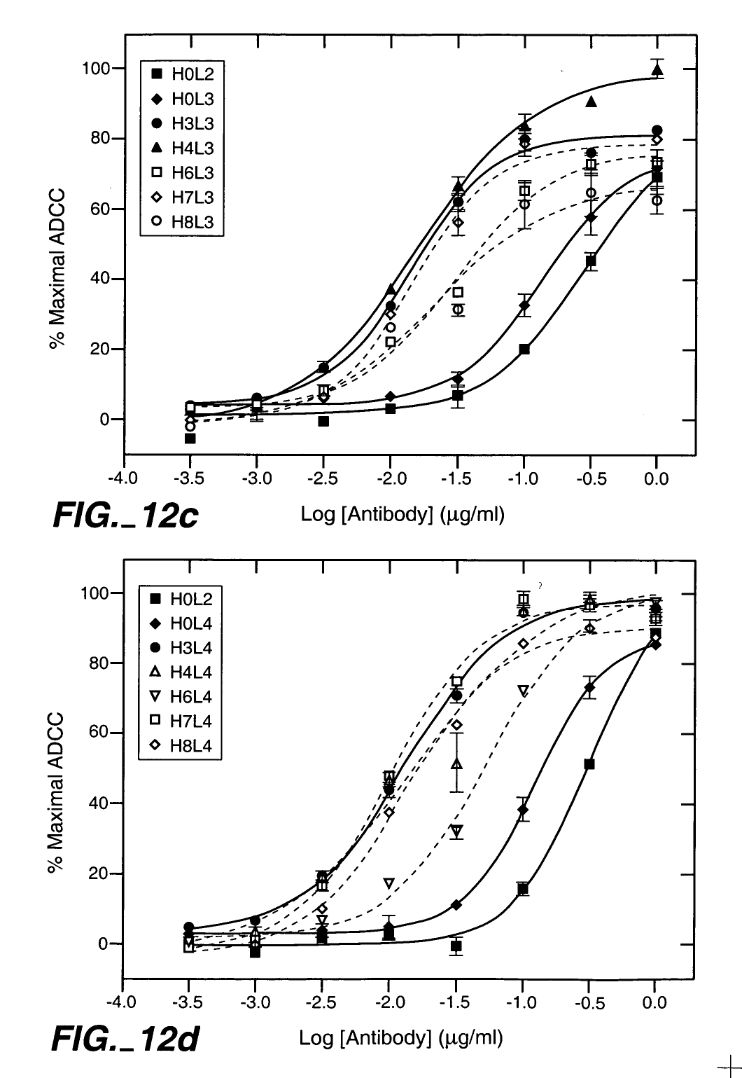

To assess the capacity of the anti-EGFR antibodies to mediate effector function against EGFR expressing cells, the C225 variants were tested in a cell-based ADCC assay. Human peripheral blood monocytes (PBMCs) were used as effector cells, A431 epidermoid carcinoma cells were used as target cells, and lysis was monitored by measuring LDH activity using the Cytotoxicity Detection Kit as described above. FIG. 12 shows the dose dependence of ADCC at various antibody concentrations for WT and variant C225 antibodies. The results show that a number of the C225 variants have comparable or better ADCC than WT C225 with respect to potency and efficacy. These data may be weighed together with the antigen affinity data and other data to choose the optimal anti-EGFR clinical candidate.

Given their lower immunogenicity relative to the WT C225 antibody, their favorable binding affinities for the EGFR target antigen, and their capacity to mediate ADCC in the presence of human effector cells, the C225 and ICR62 variants described herein may themselves be considered clinical candidates. In alternate embodiments, these sequences may be further optimized. As described in U.S. Ser. Nos. 60/527,167; 60/582,613; 60/619,483; and U.S. Ser. No. 10/______, entitled “Methods of Generating Variant Proteins with Increased Host String Content and Compositions Thereof”, filed on Dec. 6, 2004, because variant sequences of the invention are preferably derived from a HSC-increasing procedure in which substitution of structurally important positions is disallowed or discouraged, it is likely that additional optimization of HSC is possible if those positions are allowed to vary in a secondary analysis. Thus one or more subsequent substitutions may be explored to increase antigen affinity or further improve HSC, for example by mutating residues that were masked in the calculations and/or residues in or close to the CDRs or VL/VH interface. Thus, for example, the H4/L3 or H7/L4 C225 variant can be thought of as a primary variant or template for further optimization, and variants of H4/L3 or H7/L4 C225 can be thought of as secondary variants. Secondary substitutions in the variants of the present invention will comprise forward or neutral mutations with respect to human sequences, and thus are expected to only improve or to not affect HSC. An additional benefit of generating secondary variants is that, by exploring quality structural and epitope diversity, it is also possible that other properties can be optimized, including but not limited to affinity, activity, specificity, solubility, expression level, and effector function.

### Example 3

### Optimized Anti-EGFR Antibodies

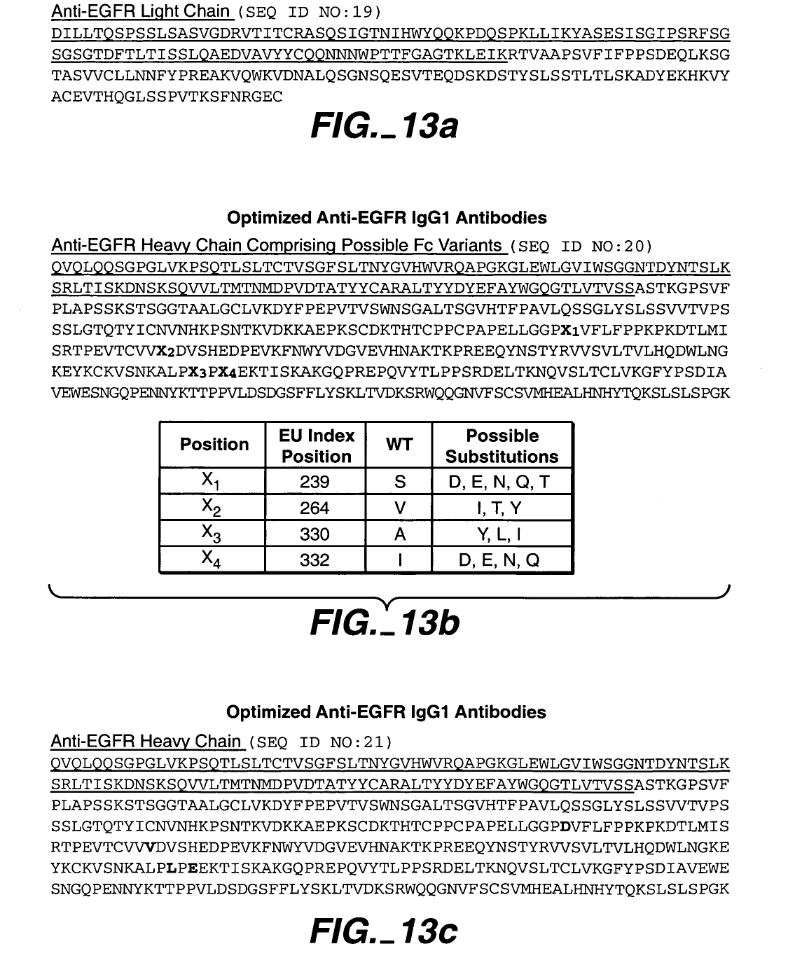

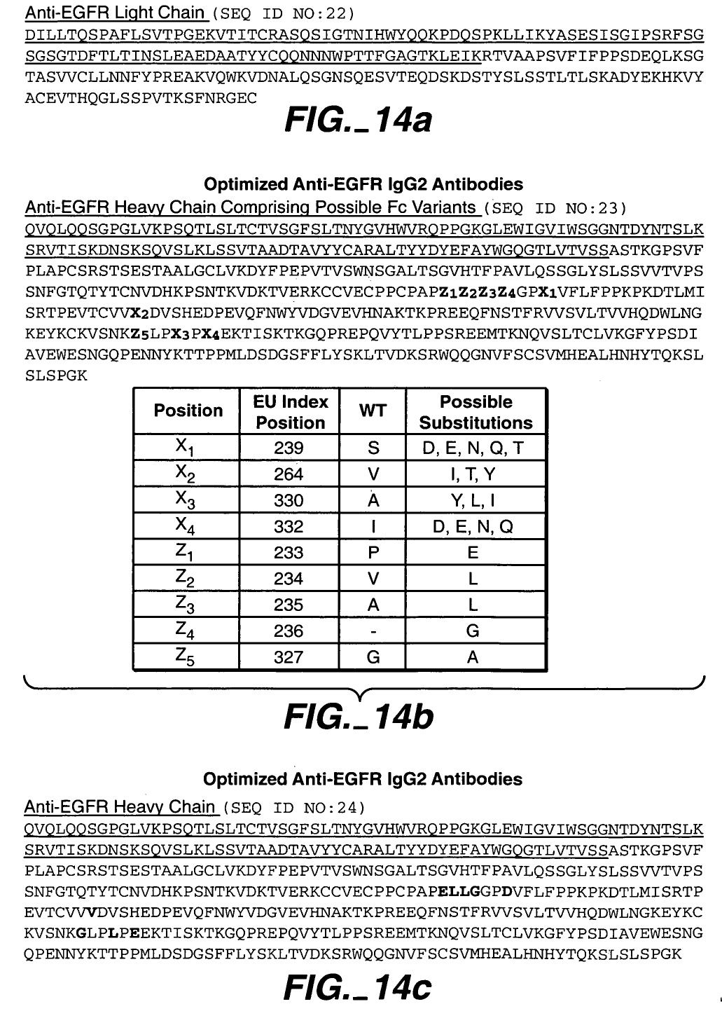

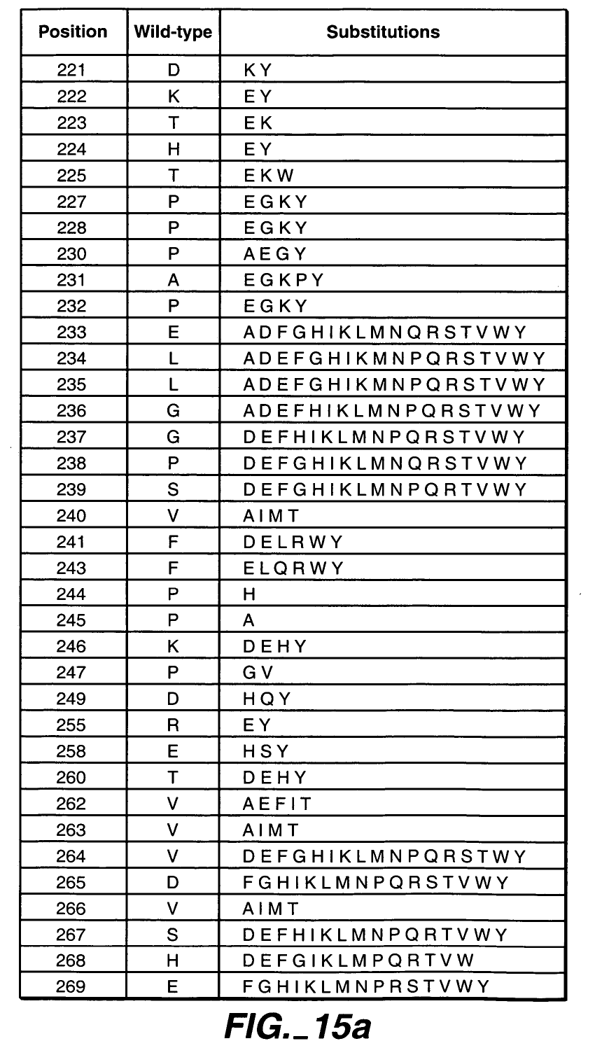

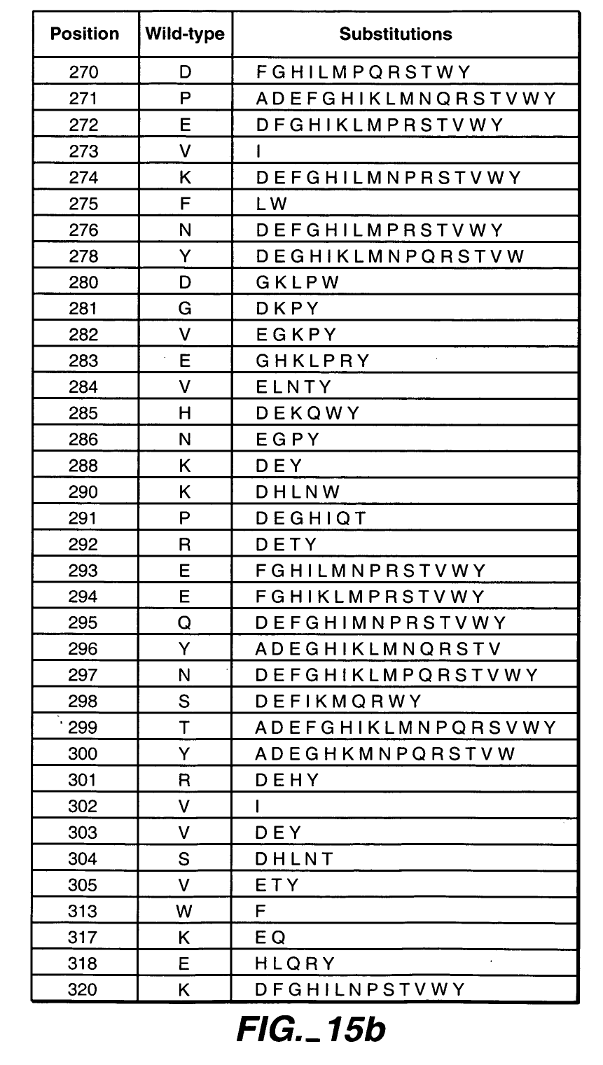

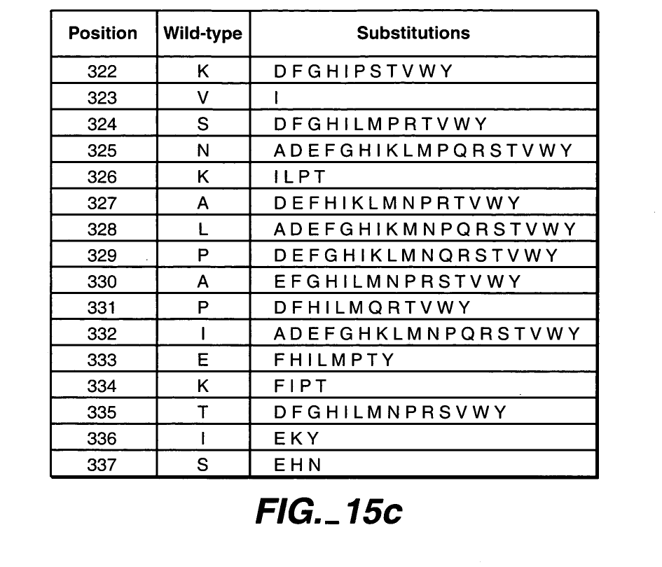

The optimal anti-EGFR clinical candidate may comprise amino acid modifications that both enhance effector function and reduce immunogenicity relative to a parent anti-EGFR protein. A variety of proteins that target EGFR are contemplated herein that comprise one or more substitutions which provide enhanced effector function, reduced immunogenicity, or both. In a preferred embodiment, the protein of the present invention comprises amino acid modifications that enhance effector function and reduce immunogenicity. FIG. 13 provides the light and heavy chain sequences of an EGFR targeting antibody that comprises H4/L3 C225, as described above, combined with a number of possible variant IgG1 constant regions that provide enhanced effector function. FIG. 14 provides the light and heavy chain sequences of an EGFR targeting antibody that comprises H7/L4 C225, as described above, combined with a number of possible variant IgG2 constant regions that provide enhanced effector function.

The sequences provided in FIGS. 13 and 14 are not meant to constrain the present invention to these examples. Other variable regions besides WT and variant C225 and ICR62 may be used to target EGFR in the context of antibodies, Fc fusions, or other proteins with optimized effector function. Alternate variable regions may be any known or unknown anti-EGFR antibody, whether they be nonhuman, chimeric, humanized, or fully human. What is important is that the variants of the present invention that provide optimized effector function may be linked with any EGFR targeting protein, be it an antibody, Fc fusion, or other protein, to provide optimal clinical properties.

The Fc modifications defined in FIGS. 13 and 14 that provide enhanced effector function are not meant to constrain the invention to only these modifications for effector function optimization. For example, as described in U.S. Pat. No. 6,737,056, PCT US2004/000643, U.S. Ser. No. 10/370,749, and PCT/US2004/005112, the substitutions S298A, S298D, K326E, K326D, E333A, K334A, and P396L provide optimized FcγR binding and/or enhanced ADCC. Furthermore, as disclosed in Idusogie et al., 2001, J. Immunology 166:2571-2572, substitutions K326W, K326Y, and E333S provide enhanced binding to the complement protein C1q and enhanced CDC. Finally, as described in Hinton et al., 2004, J. Biol. Chem. 279(8): 6213-6216, substitutions T250Q, T250E, M428L, and M428F provide enhanced binding to FcRn and improved pharmacokinetics. Modifications need not be restricted to the Fc region. It is also possible that the mutational differences in the Fab and hinge regions may provide optimized FcγR and/or C1q binding and/or effector function. For example, as disclosed in U.S. Ser. Nos. 60/556,353; 60/573,302; 60/585,328; 60/586,837; 60/589,906; 60/599,741; 60/607,398; 60/614,944, and 60/619,409, the Fab and hinge regions of an antibody may impact effector functions such as antibody dependent cell-mediated cytotoxicity (ADCC), antibody dependent cell-mediated phagocytosis (ADCP), and complement dependent cytotoxicity (CDC). Thus immunoglobulin variants comprising substitutions in the Fc, Fab, and/or hinge regions are contemplated. For example, the EGFR targeting proteins may be combined with one or more substitutions in the VL, CL, VH, CH1, and/or hinge regions. Furthermore, further modifications may be made in non IgG1 immunoglobulins to corresponding amino acids in other immunoglobulin classes to provide more optimal properties, as described in U.S. Ser. Nos. 60/621,387 and 60/629,068. For example, in one embodiment, an IgG2 antibody, similar to the antibody presented in FIG. 14, may comprise one or more modifications to corresponding amino acids in IgG1 or IgG3 CH1, hinge, CH2, and/or CH3. In another embodiment, an IgG2 antibody, similar to the antibody presented in FIG. 14, may comprise all of the IgG1 CH1 and hinge substitutions, i.e., the IgG2 variant comprises the entire CH1 domain and hinge of IgG1.

All references cited herein are expressly incorporated by reference.

Whereas particular embodiments of the invention have been described above for purposes of illustration, it will be appreciated by those skilled in the art that numerous variations of the details may be made without departing from the invention as described in the appended claims.

1. An anti-human EGFR antibody that comprises a variant human immunoglobulin G constant region comprising a sequence having the formula:
Vx(222)-Vx(223)-Vx(224)-Vx(225)-Fx(226)-Vx(227)-Vx(228)-Fx(229)-Vx(230)-Fx(231)-Vx(232)-Vx(233)-Vx(234)-Vx(235)-Vx(236)-Vx(237)-Vx(238)-Vx(239)-Vx(240)-Vx(241)-Fx(242)-Vx(243)-Vx(244)-Vx(245)-Vx(246)-Vx(247)-Fx(248-254)-Vx(255)-Fx(256-257)-Vx(258)-Fx(259)-Vx(260)-Fx(261)-Vx(262)-Vx(263)-Vx(264)-Vx(265)-Vx(266)-Vx(267)-Vx(268)-Vx(269)-Vx(270)-Vx(271)-Vx(272)-Vx(273)-Vx(274)-Vx(274)-Vx(275)-Vx(276)-Fx(277)-Vx(278)-Fx(279)-Vx(280)-Vx(281)-Vx(282)-Vx(283)-Vx(284)-Vx(285)-Vx(286)-Fx(287)-Vx(288)-Fx(289)-Vx(290)-Vx(291)-Vx(292)-Vx293)-Vx(294)-Vx(295)-Vx(296)-Vx(297)-Vx(298)-Vx(299)-Vx(300)-Vx(301)-Vx(302)-Vx(303)-Vx(304)-Vx(305)-Fx(306-312)-Vx(313)-Fx(314-316)-Vx(317)-Vx(318)-Fx(319)-Vx(320)-Fx(321)-Vx(322)-Vx(323)-Vx(324)-Vx(325)-Vx(326)-Fx(327)-Vx(328)-Vx(329)-Fx(330)-Vx(331)-Vx(332)-Vx(333)-Vx(334)-Vx(335)-Vx(336)-Vx(337)-Fx(338-447)
wherein
Vx(222) is an amino acid selected from the group consisting of K, E and Y;
Vx(223) is an amino acid selected from the group consisting of T, E and K;
Vx(224) is an amino acid selected from the group consisting of H, E and Y;
Vx(225) is an amino acid selected from the group consisting of T, E, K and W;
Fx(226) is the human wild-type IgG1 sequence at position 226;
Vx(227) is an amino acid selected from the group consisting of P, E, G, K and Y;
Vx(228) is an amino acid selected from the group consisting of P, E, G, K and Y;
Fx(229) is the human wild-type IgG1 sequence at position 229;
Vx(230) is an amino acid selected from the group consisting of P, A, E, G and Y;
Fx(231) is the human wild-type IgG1 sequence at position 231;
Vx(232) is an amino acid selected from the group consisting of P, E, G, K and Y;
Vx(233) is an amino acid selected from the group consisting of E, A, D, F, G, H, I, K, L, M, N, Q, R, S, T, V, W and Y;
Vx(234) is an amino acid selected from the group consisting of L, A, D, E, F, G, H, I, K, M, N, P, Q, R, S, T, V, W and Y;
Vx(235) is an amino acid selected from the group consisting of L, A, D, E, F, G, H, I, K, M, N, P, Q, R, S, T, V, W and Y;
Vx(236) is an amino acid selected from the group consisting of G, A, D, E, F, H, I, K, L, M, N, P, Q, R, S, T, V, W and Y;
Vx(237) is an amino acid selected from the group consisting of G, D, E, F, H, I, K, L, M, N, P, Q, R, S, T, V, W and Y;
Vx(238) is an amino acid selected from the group consisting of P, D, E, F, G, H, I, K, L, M, N, Q, R, S, T, V, W and Y;
Vx(239) is an amino acid selected from the group consisting of S, D, E, F, G, H ,I ,K, L, M, N, P, Q, R, T, V, W and Y;
Vx(240) is an amino acid selected from the group consisting of V, A, I, M and T;
Vx(241) is an amino acid selected from the group consisting of F, D, E, L, R, W and Y;
Fx(242) is the human wild-type IgG1 sequence at position 242;
Vx(243) is an amino acid selected from the group consisting of F, E, L, Q, R, W and Y;
Vx(244) is an amino acid selected from the group consisting of P and H;
Vx(245) is an amino acid selected from the group consisting of P and A;
Vx(246) is an amino acid selected from the group consisting of K, D, E, H and Y;
Vx(247) is an amino acid selected from the group consisting of P, G and V;
Fx(248-254) is the human wild-type IgG1 sequence at positions 248-254;
Vx(255) is an amino acid selected from the group consisting of R, E and Y;
Fx(256-257) is the human wild-type IgG1 sequence at positions 256-257;
Vx(258) is an amino acid selected from the group consisting of E, H, S and Y;
Fx(259) is the human wild-type IgG1 sequence at position 259;
Vx(260) is an amino acid selected from the group consisting of T, D, E, H and Y;
Fx(261) is the human wild-type IgG1 sequence at position 261;
Vx(262) is an amino acid selected from the group consisting of V, A, E, F, I and T;
Vx(263) is an amino acid selected from the group consisting of V, A, I, M and T;
Vx(264) is an amino acid selected from the group consisting of V, D, E, F, G, H, I, K, L, M, N, P, Q, R, S, T, W and Y;
Vx(265) is an amino acid selected from the group consisting of D, F, G, H, I, K, L, M, N, P, Q, R, S, T, V, W and Y;
Vx(266) is an amino acid selected from the group consisting of V, A, I, M and T;
Vx(267) is an amino acid selected from the group consisting of S, D, E, F, H, I, K, L, M, N, P, Q, R, T, V, W and Y;
Vx(268) is an amino acid selected from the group consisting of H, D, E, F, G, I, K, L, M, P, Q, R, T, V and W;
Vx(269) is an amino acid selected from the group consisting of E, F, G, H, I, K, L, M, N, P, R, S, T, V, W and Y;
Vx(270) is an amino acid selected from the group consisting of D, F, G, H, I, L, M, P, Q, R, S, T, W and Y;
Vx(271) is an amino acid selected from the group consisting of P, A, D, E, F, G, H, I, K, L, M, N, Q, R, S, T, V, W and Y;
Vx(272) is an amino acid selected from the group consisting of E, D, F, G, H, I, K, L, M, P, R, S, T, V, W and Y;
Vx(273) is an amino acid selected from the group consisting of V and I;
Vx(274) is an amino acid selected from the group consisting of K, D, E, F, G, H, I, L, M, N, P, R, S, T, V, W and Y;
Vx(275) is an amino acid selected from the group consisting of F, L and W;
Vx(276) is an amino acid selected from the group consisting of N, D, E, F, G, H, I, L, M, P, R, S, T, V, W and Y;
Fx(277) is the human wild-type IgG1 sequence at position 277;
Vx(278) is an amino acid selected from the group consisting of Y, D, E, G, H, I, K, L, M, N, P, Q, R, S, T, V and W;
Fx(279) is the human wild-type IgG1 sequence at position 279;
Vx(280) is an amino acid selected from the group consisting of D, G, K, L, P and W;
Vx(281) is an amino acid selected from the group consisting of G, D, K, P and Y;
Vx(282) is an amino acid selected from the group consisting of V, E, G, K, P and Y;
Vx(283) is an amino acid selected from the group consisting of E, G, H, K, L, P, R and Y;
Vx(284) is an amino acid selected from the group consisting of V, E, L, N, T and Y;
Vx(285) is an amino acid selected from the group consisting of H, D, E, K, Q, W and Y;
Vx(286) is an amino acid selected from the group consisting of N, E, G, P and Y;
Fx(287) is the human wild-type IgG1 sequence at position 287;
Vx(288) is an amino acid selected from the group consisting of K, D, E and Y;
Fx(289) is the human wild-type IgG1 sequence at position289;
Vx(290) is an amino acid selected from the group consisting of K, D, H, L, N and W;
Vx(291) is an amino acid selected from the group consisting of P, D, E, G, H, I, Q and T;
Vx(292) is an amino acid selected from the group consisting of R, D, E, T and Y;
Vx(293) is an amino acid selected from the group consisting of E, F, G, H, I, L, M, N, P, R, S, T, V, W and Y;
Vx(294) is an amino acid selected from the group consisting of E, F, G, H, I, K, L, M, P, R, S, T, V, W and Y;
Vx(295) is an amino acid selected from the group consisting of Q, D, E, F, G, H, I, M, N, P, R, S, T, V, W and Y;
Vx(296) is an amino acid selected from the group consisting of Y, A, D, E, G, H, I, K, L, M, N, Q, R, S, T and V;
Vx(297) is an amino acid selected from the group consisting of N, D, E, F, G, H, I, K, L, M, P, Q, R, S, T, V, W and Y;
Vx(298) is an amino acid selected from the group consisting of S, D, E, F, I, K, M, Q, R, W and Y;
Vx(299) is an amino acid selected from the group consisting of T, A, D, E, F, G, H, I, K, L, M, N, P, Q, R, S, V, W and Y;
Vx(300) is an amino acid selected from the group consisting of Y, A, D, E, G, H, K, M, N, P, Q, R, S, T, V and W;
Vx(301) is an amino acid selected from the group consisting of R, D, E, H and Y;
Vx(302) is an amino acid selected from the group consisting of V and I;
Vx(303) is an amino acid selected from the group consisting of V, D, E and Y;
Vx(304) is an amino acid selected from the group consisting of S, D, H, L, N and T;
Vx(305) is an amino acid selected from the group consisting of V, E, T and Y;
Fx(306-312) is the human wild-type IgG1 sequence at positions 306-312;
Vx(313) is an amino acid selected from the group consisting of W and F;
Fx(314-316) is the human wild-type IgG1 sequence at positions 314-316;
Vx(317) is an amino acid selected from the group consisting of K E and Q;
Vx(318) is an amino acid selected from the group consisting of E, H, L, Q, R and Y;
Fx(319) is the human wild-type IgG1 sequence at position 319;
Vx(320) is an amino acid selected from the group consisting of K, D, F, G, H, I, L, N, P, S, T, V, W and Y;
Fx(321) is the human wild-type IgG1 sequence at position 321;
Vx(322) is an amino acid selected from the group consisting of K, D, F, G, H, I, P, S, T, V, W and Y;
Vx(323) is an amino acid selected from the group consisting of V and I;
Vx(324) is an amino acid selected from the group consisting of S, D, F, G, H, I, L, M, P, R, T, V, W and Y;
Vx(325) is an amino acid selected from the group consisting of N, A, D, E, F, G, H, I, K, L, M, P, Q, R, S, T, V, W and Y;
Vx(326) is an amino acid selected from the group consisting of K, I, L, P and T;
Fx(327) is the human wild-type IgG1 sequence at position 327
Vx(328) is an amino acid selected from the group consisting of L, A, D, E, F, G, H. I, K, M, N, P, Q, R, S, T, V, W and Y;
Vx(329) is an amino acid selected from the group consisting of P, D, E, F, G, H. 1, K, L, M, N, Q, R, S, T, V, W and Y;
Fx(330) is the human wild-type IgG1 sequence at position330;
Vx(331) is an amino acid selected from the group consisting of P, D, F, H, I, L, M, Q, R, T, V, W and Y;
Vx(332) is an amino acid selected from the group consisting of I, A, D, E, F, G, H, K, L, M, N, P, Q, R, S, T, V, W and Y;
Vx(333) is an amino acid selected from the group consisting of E, F, H, I, L, M, P, T and Y;
Vx(334) is an amino acid selected from the group consisting of K, F, I, P and T;
Vx(335) is an amino acid selected from the group consisting of T, D, F, G, H, I, L, M, N, P, R, S, V, W and Y;
Vx(336) is an amino acid selected from the group consisting of I, E, K and Y;
Vx(337) is an amino acid selected from the group consisting of S, E, H and N;
Fx(338-447) is the human wild-type IgG1 sequence at positions 338-447;
wherein at least one amino acid is substituted as compared to the wild-type sequence (SEQ ID NO:X), and wherein said antibody has an altered property, said property selected from the group consisting of altered FcγR binding and altered effector function.

2. An anti-human EGFR antibody according to claim 1 wherein said IgG region is an IgG1 region having the formula:
Fx(222-238)-Vx(239)-Fx(240-263)-Vx(264)-Fx(265-329)-Vx(330)-Fx(331 )-Vx(332)-Fx(333-447)
Wherein
Fx(222-238) is the human wild-type IgG1 sequence at positions 222-238;
Vx(239) is an amino acid selected from the group consisting of S, D, E, F, G, H, I, K, L, M, N, P, Q, R, T, V, W and Y;
Fx(240-263) is the human wild-type IgG1 sequence at positions 240-263;
Vx(264) is an amino acid selected from the group consisting of V, D, E, F, G, H, I, K, L, M, N, P, Q, R, S, T, W and Y;
Fx(265-329) is the human wild-type IgG1 sequence at positions 265-229;
Vx(330) is an amino acid selected from the group consisting of A, E, F, G, H, I, L, M, N, P, R, S, T, V, W and Y;
Fx(3 31) is the human wild-type IgG1 sequence at position 331;
Vx(332) is an amino acid selected from the group consisting of I, A, D, E, F, G, H, K, L, M, N, P, Q, R, S, T, V, W and Y
Fx(333-447) is the human wild-type IgG1 sequence at positions 333-447;
And wherein at least one of said Vx amino acids is not the wild-type amino acid.

3. An anti-human EGFR antibody according to claim 1 wherein said IgG region is an IgG2 region having the formula:
Fx(222-232)-Vx(233)-Vx(234)-Vx(235)-Vx(236)-Fx(237-238)-Vx(239)-Fx(240-263)-Vx(264)-Fx(265-326)-Vx(327)-Fx(328-329)-Vx(330)-Fx(331 )-Vx(332)-Fx(333-447)
Wherein
Fx(222-232) is the human wild-type IgG2 sequence at positions 222-232;
Vx(233) is an amino acid selected from the group consisting of E, A, D, F, G, H, I, K, L, M, N, Q, R, S, T, V, W and Y;
Vx(234) is an amino acid selected from the group consisting of L, A, D, E, F, G, H, I, K, M, N, P, Q, R, S, T, V, W and Y;
Vx(235) is an amino acid selected from the group consisting of L, A, D, E, F, G, H, I, K, M, N, P, Q, R, S, T, V, W and Y;
Vx(236) is an amino acid deletion as compared to the EU Kabat sequence;
Fx(237-238) is the human wild-type IgG2 sequence at positions 237-238;
Vx(239) is an amino acid selected from the group consisting of S, D, E, F, G, H, I, K, L, M, N, P, Q, R, T, V, W and Y;
Fx(240-263) is the human wild-type IgG2 sequence at positions 240-263;
Vx(264) is an amino acid selected from the group consisting of V, D, E, F, G, H, I, K, L, M, N, P, Q, R, S, T, W and Y;
Fx(265-326) is the human wild-type IgG2 sequence at positions 265-326;
Vx(327) is an amino acid selected from the group consisting of A, D, E, F, H, I, K, L, M, N, P, R, T, V, W and Y;
Fx(328-329) is the human wild-type IgG2 sequence at positions 328-329;
Vx(330) is an amino acid selected from the group consisting of A, E, F, G, H, I, L, M, N, P, R, S, T, V, W and Y;
Fx(3 31) is the human wild-type IgG2 sequence at position 331;
Vx(332) is an amino acid selected from the group consisting of I, A, D, E, F, G, H, K, L, M, N, P, Q, R, S, T, V, W and Y;
Fx(333-447) is the human wild-type IgG2 sequence at positions 333-447;
and wherein at least one of said Vx amino acids is not the wild-type amino acid.

4. An antibody according to any of claims 1-3 wherein said antibody comprises said IgG region and a variable light region selected from the group consisting of C225 L0 (SEQ ID NO:2), C225 L2 (SEQ ID NO:7), C225 L3 (SEQ ID NO:8), C225 L4 (SEQ ID NO:9) and ICR62 L3 (SEQ ID NO:16).

5. An antibody according to any of claims 1-4 wherein said antibody comprises said IgG region and a variable heavy region selected from the group consisting of C225 H0 (SEQ ID NO:3, C225 H3 (SEQ ID NO:10), C225 H4 (SEQ ID NO:11), C225 H5 (SEQ ID NO:12), C225 H6 (SEQ ID NO:13), C225 H7 (SEQ ID NO:14), C225 H8 (SEQ ID NO:15), ICR62 H9 (SEQ ID NO:17) and ICR62 H10 (SEQ ID NO:18).

6. An antibody according to any of claims 1-5 wherein said antibody comprises an engineered glycoform.


SEQUENCE LISTING (25 sequences)

SEQ ID NO: 1 - protein, 330 aa, Homo sapiens [sequence omitted]

SEQ ID NO: 2 - protein, 107 aa, Homo sapiens
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 3 - protein, 119 aa, Homo sapiens
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 4 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 106 aa, Homo sapiens
DIQMTQSPSFLSASVGDRVTINCKASQNINNYLNWYQQKLGEAPKRLIYNTNNLQTGIPSRFSGSGSGTDYTLTISSLQPEDFATYFCLQHNSFPTFGAGTKLELK

SEQ ID NO: 6 - protein, 120 aa, Homo sapiens
QVNLLQSGAALVKPGASVKLSCKGSGFTFTDYKIHWVKQSHGKSLEWIGYFNPNSGYSTYNEKFKSKATLTADKSTDTAYMELTSLTSEDSATYYCTRLSPGGYYVMDAWGQGASVTVSS

SEQ ID NO: 7 - protein, 107 aa, Artificial
  synthetic
DILLTQSPATLSLSPGERVTLSCRASQSIGTNIHWYQQKPDQSPKLLIKYASESISGIPSRFSGSGSGTDFTLTISSLQPEDFADYYCQQNNNWPTTFGAGTKLEIK

SEQ ID NO: 8 - protein, 107 aa, Artificial
  synthetic
DILLTQSPSSLSASVGDRVTITCRASQSIGTNIHWYQQKPDQSPKLLIKYASESISGIPSRFSGSGSGTDFTLTISSLQAEDVAVYYCQQNNNWPTTFGAGTKLEIK

SEQ ID NO: 9 - protein, 107 aa, Artificial
  synthetic
DILLTQSPAFLSVTPGEKVTITCRASQSIGTNIHWYQQKPDQSPKLLIKYASESISGIPSRFSGSGSGTDFTLTINSLEAEDAATYYCQQNNNWPTTFGAGTKLEIK

SEQ ID NO: 10 - protein, 119 aa, Artificial
  synthetic
QVQLQQSGPGLVKPSQTLSLTCTVSGFSLTNYGVHWVRQAPGKGLEWLGVIWSGGNTDYNTSLKSRLTISKDNSKSQVVLQMNSLRAEDTAVYYCARALTYYDYEFAYWGQGTLVTVSS

SEQ ID NO: 11 - protein, 119 aa, Artificial
  synthetic
QVQLQQSGPGLVKPSQTLSLTCTVSGFSLTNYGVHWVRQAPGKGLEWLGVIWSGGNTDYNTSLKSRLTISKDNSKSQVVLTMTNMDPVDTATYYCARALTYYDYEFAYWGQGTLVTVSS

SEQ ID NO: 12 - protein, 119 aa, Artificial
  synthetic
QVQLQQSGPGLVKPSQTLSLTCTVSGFSLTNYGVHWVRQAPGKGLEWLGVIWSGGNTDYNTPLTSRLTINKDNSKSQVVLQMNSLRAEDTAVYYCARALTYYDYEFAYWGQGTLVTVSS

SEQ ID NO: 13 - protein, 119 aa, Artificial
  synthetic
QVQLQQSGPGLVKPSQTLSLTCTVSGFSLTNYGVHWVRQAPGKGLEWVGVIWSGGNTDYNTSVKGRFTISKDNSKSQVYLQMNSLRAEDTAVYYCARALTYYDYEFAYWGQGTLVTVSS

SEQ ID NO: 14 - protein, 119 aa, Artificial
  synthetic
QVQLQQSGPGLVKPSQTLSLTCTVSGFSLTNYGVHWVRQPPGKGLEWIGVIWSGGNTDYNTSLKSRVTISKDNSKSQVSLKLSSVTAADTAVYYCARALTYYDYEFAYWGQGTLVTVSS

SEQ ID NO: 15 - protein, 119 aa, Artificial
  synthetic
QVQLVESGGGLVQPGRSLRLSCAVSGFSLTNYGVHWVRQAPGKGLEWVSVIWSGGNTDYNTSVKGRFTISKDNSKSTVYLQMNSLRAEDTAVYYCARALTYYDYEFAYWGQGTLVTVSS

SEQ ID NO: 16 - protein, 106 aa, Artificial
  synthetic
DIQMTQSPSSLSASVGDRVTITCRASQNINNYLNWYQQKPGKAPKRLIYNTNNLQTGIPSRFSGSGSGTDYTLTISSLQPEDFATYFCLQHNSFPTFGAGTKLEIK

SEQ ID NO: 17 - protein, 120 aa, Artificial
  synthetic
QVQLQQSGPGLVKPGASVKVSCKGSGFTFTDYKIHWVRQAPGKSLEWMGYFNPNSGYSTYNEKFQGRVTITADKSTDTAYMELSSLRSEDTAVYYCTRLSPGGYYVMDAWGQGTLVTVSS

SEQ ID NO: 18 - protein, 120 aa, Artificial
  synthetic
QVQLVQSGAEVKKPGASVKVSCKGSGFTFTDYKIHWVRQAPGKSLEWMGYFNPNSGYSTYNEKFQGRVTITADKSTDTAYMELSSLRSEDTAVYYCTRLSPGGYYVMDAWGQGTLVTVSS

SEQ ID NO: 19 - protein, 214 aa, Artificial
  synthetic
DILLTQSPSSLSASVGDRVTITCRASQSIGTNIHWYQQKPDQSPKLLIKYASESISGIPSRFSGSGSGTDFTLTISSLQAEDVAVYYCQQNNNWPTTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 449 aa, Artificial
  synthetic
QVQLQQSGPGLVKPSQTLSLTCTVSGFSLTNYGVHWVRQAPGKGLEWLGVIWSGGNTDYNTSLKSRLTISKDNSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPXVFLFPPKPKDTLMISRTPEVTCVVXDVSHEDPEVKFNWYVDGVEVHNAKTKPREEQYNSTYRVVSVLTVLHQDWLNGKEYKCKVSNKALPXPXEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 449 aa, Artificial
  synthetic
QVQLQQSGPGLVKPSQTLSLTCTVSGFSLTNYGVHWVRQAPGKGLEWLGVIWSGGNTDYNTSLKSRLTISKDNSKSQVVLTMTNMDPVDTATYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial
  synthetic
DILLTQSPAFLSVTPGEKVTITCRASQSIGTNIHWYQQKPDQSPKLLIKYASESISGIPSRFSGSGSGTDFTLTINSLEAEDAATYYCQQNNNWPTTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 446 aa, Artificial
  synthetic
QVQLQQSGPGLVKPSQTLSLTCTVSGFSLTNYGVHWVRQPPGKGLEWIGVIWSGGNTDYNTSLKSRVTISKDNSKSQVSLKLSSVTAADTAVYYCARALTYYDYEFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPXXXXGPXVFLFPPKPKDTLMISRTPEVTCVVXDVSHEDPEVQFNWYVDGVEVHNAKTKPREEQFNSTFRVVSVLTVVHQDWLNGKEYKCKVSNKXLPXPXEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 446 aa, Artificial
  synthetic
QVQLQQSGPGLVKPSQTLSLTCTVSGFSLTNYGVHWVRQPPGKGLEWIGVIWSGGNTDYNTSLKSRVTISKDNSKSQVSLKLSSVTAADTAVYYCARALTYYDYEFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPDVFLFPPKPKDTLMISRTPEVTCVVVDVSHEDPEVQFNWYVDGVEVHNAKTKPREEQFNSTFRVVSVLTVVHQDWLNGKEYKCKVSNKGLPLPEEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 5 aa, Artificial
  linker
GGGGS